(19) United States
(12) Patent Application Publication
(10) Pub. No.: US 2018/0066026 A1
(43) Pub. Date: Mar. 8, 2018

(54) MODULATION OF YEP6 GENE EXPRESSION TO INCREASE YIELD AND OTHER RELATED TRAITS IN PLANTS

(71) Applicants: EI DU PONT DE NEMOURS AND COMPANY, Wilmington, DE (US); UNIVERSITY OF ILLINOIS/URBANA, Urbana (IL); PIONEER HI-BRED INTERNATIONAL, INC., Johnston, IA (US)

(72) Inventors: KEVIN FENGLER, JOHNSTON, IA (US); RAJEEV GUPTA, JOHNSTON, IA (US); BAILIN LI, JOHNSTON, IA (US); STEPHEN P MOOSE, BONDVILLE, IL (US); BENJAMIN WEERS, JOHNSTON, IA (US); WENGANG ZHOU, SAN MATEO, CA (US)

(21) Appl. No.: 15/625,885

(22) PCT Filed: Dec. 3, 2015
(86) PCT No.: PCT/US2015/063639

§ 371 Date: Jun. 16, 2017

Related U.S. Application Data
(60) Provisional application No. 62/092,933, filed on Dec. 17, 2014.

(51) Int. Cl. C07K 14/415; A01H 5/10; C12N 15/82

(52) U.S. Cl. CPC C07K 14/415; C12N 15/8273; A01H 5/10

(57) ABSTRACT

Nucleotide sequences encoding YEP6 polypeptides are provided herein, along with plants and cells having reduced levels of YEP6 gene expression, reduced levels of YEP6 polypeptide activity, or both. Plants with reduced levels of gene expression of at least one YEP6 gene and/or reduced levels of YEP6 polypeptide activity that exhibit increased yield, increased staygreen, increased abiotic stress tolerance, or any combination of these, are provided. Methods for increasing yield, staygreen and abiotic stress tolerance in plants, by modulating YEP6 gene expression or activity, are also provided.

### CROSS REFERENCE TO RELATED APPLICATION

This application claims the benefit of U.S. National Application No. 62/092,933, filed Dec. 17, 2014, which is incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 20151117_RTS10881 APCT_ST25 created on Nov. 17, 2015 and having a size of 237 kilobytes and is filed concurrently with the specification. The sequence listing contained in this ASCII formatted document is part of the specification and is herein incorporated by reference in its entirety.

### FIELD

The field relates to plant breeding and genetics and, in particular, to recombinant DNA constructs useful in plants for increasing yield and/or conferring tolerance to abiotic stress tolerance.

### BACKGROUND

Yield is a trait of particular economic interest, especially because of increasing world population and the dwindling supply of arable land available for agriculture. Crops such as corn, wheat, rice, canola and soybean account for over half the total human caloric intake, whether through direct consumption of the seeds themselves or through consumption of meat products raised on processed seeds.

Several factors contribute to crop yield. One approach to increase crop yield is to extend the duration of active photosynthesis. The staygreen phenotype has been associated with increases in crop yield. Plants assimilate carbohydrates and nitrogen in vegetative organs (source) and remobilize them to newly developing tissues during development, or to reproductive organs (sink) during senescence. Increasing source strength in cereal crops can lead to increase in grain yield. Staygreen trait (or delayed senescence) during the final stage of leaf development is considered an important trait in increasing source strength in grain production. Staygreen is broadly categorized into two groups, functional and nonfunctional. Functional staygreen is defined as retaining both greenness and photosynthetic competence much longer during senescence.

Functional staygreen trait has been shown to be associated with the transition from the carbon (C) capture to the nitrogen (N) mobilization phase of foliar development (Thomas and Ougham J Exp Bot, Vol. 65, No. 14, pp. 3889-3900, 2014, Yoo et al (2007) Mol. Cells Vol. 24 (1), pp. 83-94; Thomas and Howarth (2000) J Exp Bot, (51) 329-337; Avila-Ospina et al (2014) J Exp Bot, Vol. 65 (14):3799-3811. In functional staygreen plants, the C—N transition point is delayed, or the transition occurs on time but subsequent yellowing and N remobilization occur slowly. This would indicate that the leaf senescence initiation occurs on schedule but leaf photosynthetic rate and chlorophyll content decrease much more slowly during senescence.

Functional senescence has also been shown to be a valuable trait for improving crop stress tolerance. Retention of green leaf area in staygreen genotypes in some crop plants has been associated with enhanced capacity to continue normal grain fill under drought conditions, high stem carbohydrate content and high grain weight.

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203).

Among the various abiotic stresses, drought is the major factor that limits crop productivity worldwide. Reviews on the molecular mechanisms of abiotic stress responses and the genetic regulatory networks of drought stress tolerance have been published (Valliyodan, B., and Nguyen, H. T., (2006) Curr. Opin. Plant Biol. 9:189-195; Wang, W., et al. (2003) Planta 218:1-14); Vinocur, B., and Altman, A. (2005) Curr. Opin. Biotechnol. 16:123-132; Chaves, M. M., and Oliveira, M. M. (2004) J. Exp. Bot. 55:2365-2384; Shinozaki, K., et al. (2003) Curr. Opin. Plant Biol. 6:410-417; Yamaguchi-Shinozaki, K., and Shinozaki, K. (2005) Trends Plant Sci. 10:88-94).

Another abiotic stress that can limit crop yields is low nitrogen stress. The adsorption of nitrogen by plants plays an important role in their growth (Gallais et al., J. Exp. Bot. 55(396):295-306 (2004)). Plants synthesize amino acids from inorganic nitrogen in the environment. Consequently, nitrogen fertilization has been a powerful tool for increasing the yield of cultivated plants, such as maize and soybean. If the nitrogen assimilation capacity of a plant can be increased, then increases in plant growth and yield increase are also expected. In summary, plant varieties that have better nitrogen use efficiency (NUE) are desirable.

### SUMMARY

The present disclosure includes:

One embodiment is a plant in which expression of an endogenous YEP6 gene is reduced, when compared to a control plant, wherein the YEP6 gene encodes a YEP6 polypeptide and wherein the plant exhibits at least one phenotype selected from the group consisting of: increased yield, increased abiotic stress tolerance, increased staygreen, and increased biomass compared to the control plant.

Another embodiment is a plant in which activity of an endogenous YEP6 polypeptide is reduced, when compared to the activity of wild-type YEP6 polypeptide in a control plant, wherein the plant exhibits at least one phenotype selected from the group consisting of: increased yield, increased abiotic stress tolerance, increased staygreen, and increased biomass compared to the control plant.

The plant may exhibit increased abiotic stress tolerance, and the abiotic stress may be drought stress, low nitrogen stress, or both. The plant may exhibit the phenotype of increased yield under non-stress or stress conditions. The plant may exhibit the phenotype under drought stress conditions.

The endogenous YEP6 polypeptide may comprise an amino acid sequence with at least 80% sequence identity to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98.

The plant may be a monocot plant such as but not limited to a maize plant.

The reduction in expression of the endogenous YEP6 gene may be caused by sense suppression, antisense suppression, miRNA suppression, ribozymes, or RNA interference. The reduction in expression of the endogenous YEP6 gene may also be caused by a mutation in the endogenous YEP6 gene, and the mutation may be caused by insertional mutagenesis including but not limited to transposon mutagenesis.

The activity of the endogenous YEP6 polypeptide may be reduced as a result of mutation of the endogenous YEP6 gene. The mutation may be detected using the TILLING method.

Another embodiment is a suppression DNA construct comprising a polynucleotide, wherein the polynucleotide is operably linked to a heterologous promoter in sense or antisense orientation, or both, wherein the construct is effective for reducing expression of an endogenous YEP6 gene in a plant, and wherein the polynucleotide comprises: (a) the nucleotide sequence of SEQ ID NO:1, 3, 5, 7, 9, 11, 13, 15, 17, 19, 21, 23, 25, 27, 29, 31, 33, 35, 37, 39, 41, 43, 45, 47 or 49; (b) a nucleotide sequence that has at least 80% sequence identity, when compared to SEQ ID NO:1, 3, 5, 7, 9, 11, 13, 15, 17, 19, 21, 23, 25, 27, 29, 31, 33, 35, 37, 39, 41, 43, 45, 47 or 49; (c) a nucleotide sequence of at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49; (d) a nucleotide sequence that can hybridize under stringent conditions with the nucleotide sequence of (a); or (e) a modified plant miRNA precursor, wherein the precursor has been modified to replace the miRNA encoding region with a sequence designed to produce a miRNA directed to SEQ ID NO:1, 3, 5, 7, 9, 11, 13, 15, 17, 19, 21, 23, 25, 27, 29, 31, 33, 35, 37, 39, 41, 43, 45, 47 or 49.

The polynucleotide of the suppression DNA construct may comprise at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49, and the suppression DNA construct is designed for RNA interference, and is effective for reducing expression of YEP6 gene in a plant. The polynucleotide may comprise a nucleotide sequence that has at least 90% sequence identity to SEQ ID NO:55.

Another embodiment is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of introducing into a plant a suppression DNA construct comprising a polynucleotide operably linked to a heterologous promoter, wherein the suppression DNA construct is effective for reducing expression of an endogenous YEP6 gene. The suppression DNA construct may be selected from the group consisting of: sense suppression construct, antisense suppression construct, ribozyme construct, RNA interference construct, and an miRNA construct. The suppression DNA construct may be an RNA interference construct and the RNA interference construct may comprise at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49. The RNA interference construct may comprise a polynucleotide sequence that has at least 90% sequence identity to SEQ ID NO:55.

Another embodiment is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of: (a) introducing a mutation into an endogenous YEP6 gene; and (b) detecting said mutation using the Targeted Induced Local Lesions In Genomics (TILLING) method, wherein said mutation results in reducing expression of the endogenous YEP6 gene.

Another embodiment is a method of enhancing seed yield in a plant, when compared to a control plant, wherein the plant exhibits enhanced yield under either stress conditions, or non-stress conditions, or both, the method comprising the step of reducing expression of the endogenous YEP6 gene in a plant.

Another embodiment is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the step of utilizing a transposon to introduce an insertion into an endogenous YEP6 gene in a plant, wherein the insertion is effective for reducing expression of an endogenous YEP6 gene.

Another embodiment is a method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing into a plant a suppression DNA construct comprising a polynucleotide operably linked to a heterologous promoter, wherein the polynucleotide encodes a fragment or a variant of a polypeptide having an amino acid sequence of at least 80% sequence identity, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the fragment or the variant confers a dominant-negative phenotype in the plant.

Another embodiment is a method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing a mutation in an endogenous YEP6 gene, wherein the mutation is effective for reducing the activity of the endogenous YEP6 polypeptide. The method may further comprise the step of detecting the mutation and the detection may be done using the Targeted Induced Local Lesions IN Genomics (TILLING) method.

Another embodiment is a plant obtained by any of the methods disclosed herein, wherein the plant exhibits at least one phenotype selected from the group consisting of: increased yield, increased staygreen, increased abiotic stress tolerance and increased biomass, compared to the control plant.

Another embodiment is a plant comprising any of the suppression DNA constructs disclosed herein, wherein expression of the endogenous YEP6 gene is reduced in the plant, when compared to a control plant, and wherein the plant exhibits a phenotype selected from the group consisting of: increased yield, increased staygreen, increased abiotic stress tolerance and increased biomass, compared to the control plant. The plant may exhibit an increase in abiotic stress tolerance, and the abiotic stress may be drought stress, low nitrogen stress, or both. The plant may exhibit the phenotype of increased yield and the phenotype may be exhibited under non-stress or stress conditions. The plant may be a monocot plant such as but not limited to a maize plant.

Another embodiment is a method of identifying one or more alleles associated with increased yield in a population of maize plants, the method comprising the steps of: (a) detecting in a population of maize plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, or a sequence that is 90% identical to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the one or more polymorphisms in the genomic region encoding the polypeptide or in the regulatory region controlling expression of the polypeptide is associated with yield; and (b) identifying one or more alleles at the one or more polymorphisms that are associated with increased yield. The one or more alleles associated with increased yield may be used for marker assisted selection of a maize plant with increased yield. The one or more polymorphisms may be in the coding region of the polynucleotide. The regulatory region may be a promoter element.

Another embodiment is a method of identifying one or more trait loci or a gene controlling such trait loci, the method comprising: (a) developing a breeding population of maize plants, wherein the breeding population is generated by crossing a first maize inbred line characterized as a high protein line with a second maize inbred line characterized as a low protein line; (b) selecting a plurality of progeny maize plants based on at least one phenotype of interest selected from the group consisting of delayed senescence, increased nitrogen use efficiency, increased yield, increased abiotic stress tolerance, increased staygreen, and increased biomass; (c) performing marker analysis for the one or more phenotypes identified in the progeny of plants; and (d) identifying the trait loci or the gene controlling the trait loci.

Any progeny or seeds obtained from the plants disclosed herein are also provided herein.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows the schematic of the RNA interference (RNAi) construct used for downregulation of ZmYEP6 gene in maize plants.

FIGS. 2A-2J show the alignment of the YEP6 polypeptides from Zea mays clustered in clade 1 (shown in FIG. 4 and Table 1) of the phylogenetic tree for maize YEP6 polypeptides disclosed herein this application (SEQ ID NOS:2, 4, 6, 8, 10, 12, 14, 16, 18, 20, 22, 24, 26, 28, 30, 32, 34, 36, 38, 40, 42, 44, 46, 48 and 50). FIGS. 3A through 3D show the percent sequence identity and the divergence values for each pair of amino acids sequences of YEP6 polypeptides displayed in FIG. 2A-2H. Percent similarity scores are shown in bold, while the percent divergence scores are shown in italics.

FIG. 4 shows the phylogenetic tree for all NAC proteins. ZmYEP6 and all the other YEP6 polypeptides disclosed herein are clustered in clade 1 (development clade).

FIGS. 5A-5C show the yield analysis of maize lines transformed with PHP52729. FIG. 5A shows the yield analysis at six normal nitrogen locations. FIG. 5B shows the yield analysis at three low nitrogen locations. FIG. 5C shows the yield analysis across locations for the low nitrogen locations, normal nitrogen locations, and all locations.

FIGS. 6A-6E show the yield analysis of maize lines transformed with PHP52729 for a second consecutive year. FIGS. 6A and 6B show the yield analysis at eight normal nitrogen locations, for tester 1 and tester 2, respectively. FIGS. 6C and 6D show the yield analysis at three low nitrogen locations, for tester 1 and tester 2, respectively. FIG. 6E shows the yield analysis across locations for the normal nitrogen locations, for tester 1 and tester 2.

FIG. 7 shows the results of a senescence assay done in field pots (as explained in Example 8), for different events comprising PHP52729.

FIGS. 8A-8C show the staygreen analysis of maize lines transformed with PHP52729 that were grown in the field. FIG. 8A shows the staygreen analysis for tester 1, under normal nitrogen and low nitrogen conditions across all locations.

FIG. 8B shows the staygreen analysis for tester 2, under normal nitrogen and low nitrogen conditions and across all locations. FIG. 8C shows the multitester staygreen analysis, cumulative for both the testers, tester 1 and tester 2, under normal nitrogen and low nitrogen conditions and across locations.

FIG. 9 shows the expression of ZmYEP6 in leaves of maize plants from different stages of maturity (10DAP-39 DAP).

SEQ ID NO:1 is the CDS sequence of the Zea mays YEP6 (ZmYEP6) gene, encoding a YEP6 polypeptide from Zea mays.

SEQ ID NO:2 corresponds to the amino acid sequence of Zea mays YEP6 polypeptide (ZmYEP6) encoded by SEQ ID NO:1.

Table 1 presents SEQ ID NOs for the CDS sequences of other YEP6 family members from Zea mays. The SEQ ID NOs for the corresponding amino acid sequences encoded by the cDNAs are also presented.

TABLE 1
CDS sequences Encoding Maize YEP6 Polypeptides

| | | SEQ ID NO: | SEQ ID NO: |
|---|---|---|---|
| Plant | Clone Designation | (Nucleotide) | (Amino Acid) |
| Corn | ZmYEP6-1 | 3 | 4 |
| Corn | ZmYEP6-2 | 5 | 6 |
| Corn | ZmYEP6-3 | 7 | 8 |
| Corn | ZmYEP6-4 | 9 | 10 |
| Corn | ZmYEP6-5 | 11 | 12 |
| Corn | ZmYEP6-6 | 13 | 14 |
| Corn | ZmYEP6-7 | 15 | 16 |
| Corn | ZmYEP6-8 | 17 | 18 |
| Corn | ZmYEP6-9 | 19 | 20 |
| Corn | ZmYEP6-10 | 21 | 22 |
| Corn | ZmYEP6-11 | 23 | 24 |
| Corn | ZmYEP6-12 | 25 | 26 |
| Corn | ZmYEP6-13 | 27 | 28 |
| Corn | ZmYEP6-14 | 29 | 30 |
| Corn | ZmYEP6-15 | 31 | 32 |
| Corn | ZmYEP6-16 | 33 | 34 |
| Corn | ZmYEP6-17 | 35 | 36 |
| Corn | ZmYEP6-18 | 37 | 38 |
| Corn | ZmYEP6-19 | 39 | 40 |
| Corn | ZmYEP6-20 | 41 | 42 |
| Corn | ZmYEP6-21 | 43 | 44 |
| Corn | ZmYEP6-22 | 45 | 46 |
| Corn | ZmYEP6-23 | 47 | 48 |
| Corn | ZmYEP6-24 | 49 | 50 |

*The “Full-Insert Sequence” (“FIS”) is the sequence of the entire cDNA insert.

SEQ ID NO:51 is the sequence of the forward primer for one of the markers flanking the locus encoding ZmYEP6 polypeptide, as described in Example 1 (3NR_29F).

SEQ ID NO:52 is the sequence of the reverse primer for one of the markers flanking the locus encoding ZmYEP6 polypeptide, as described in Example 1 (3NR_29R).

SEQ ID NO:53 is the sequence of the forward primer for one of the markers flanking the locus encoding ZmYEP6 polypeptide, as described in Example 1 (3NR_72F).

SEQ ID NO:54 is the sequence of the reverse primer for one of the markers flanking the locus encoding ZmYEP6, as described in Example 1 (3NR_72R).

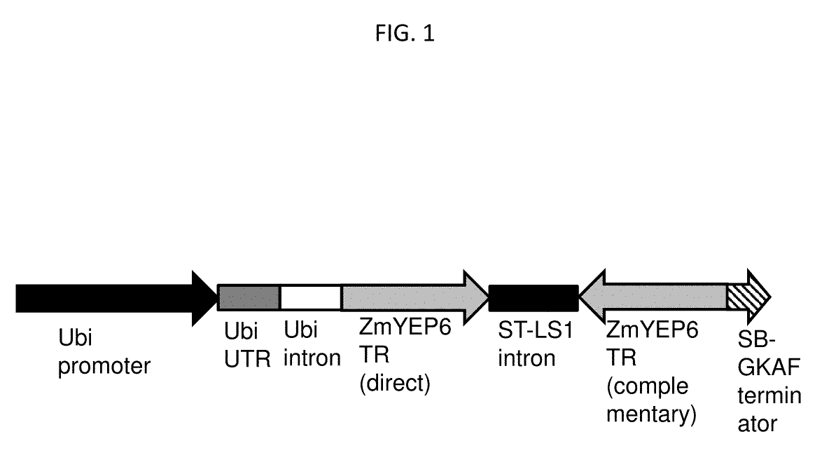

SEQ ID NO:55 is the sequence of the fragment of ZmYEP6 nucleotide sequence that was used in the RNAi construct (FIG. 1) to suppress ZmYEP6 gene expression.

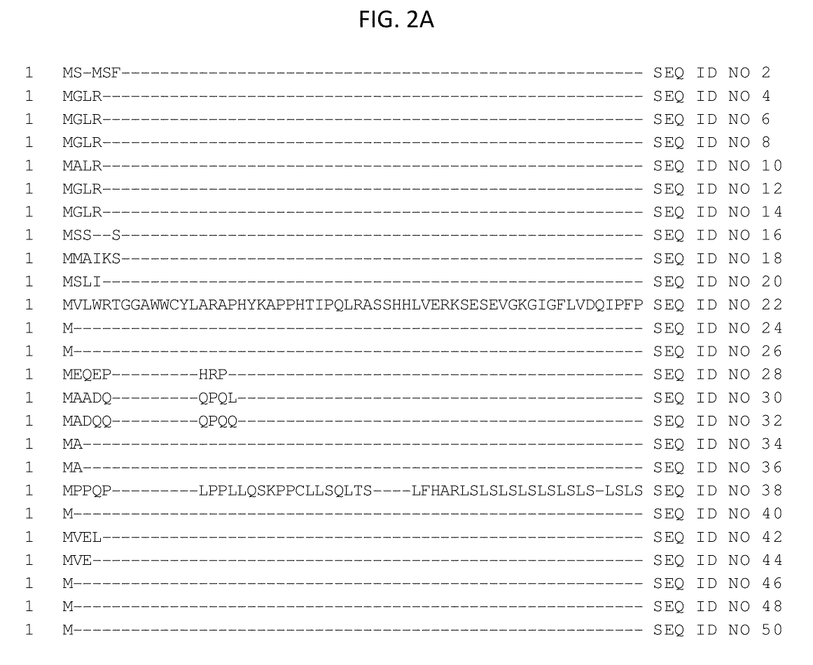

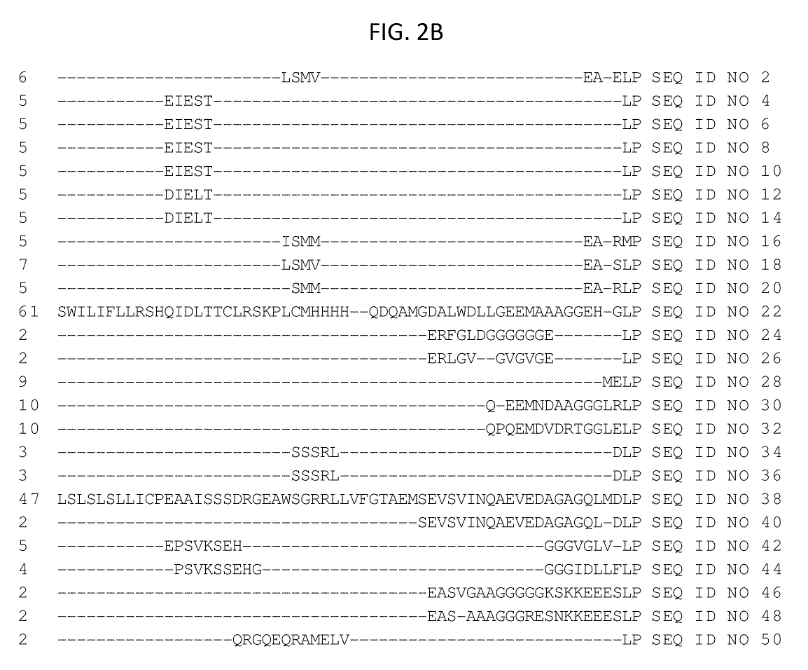

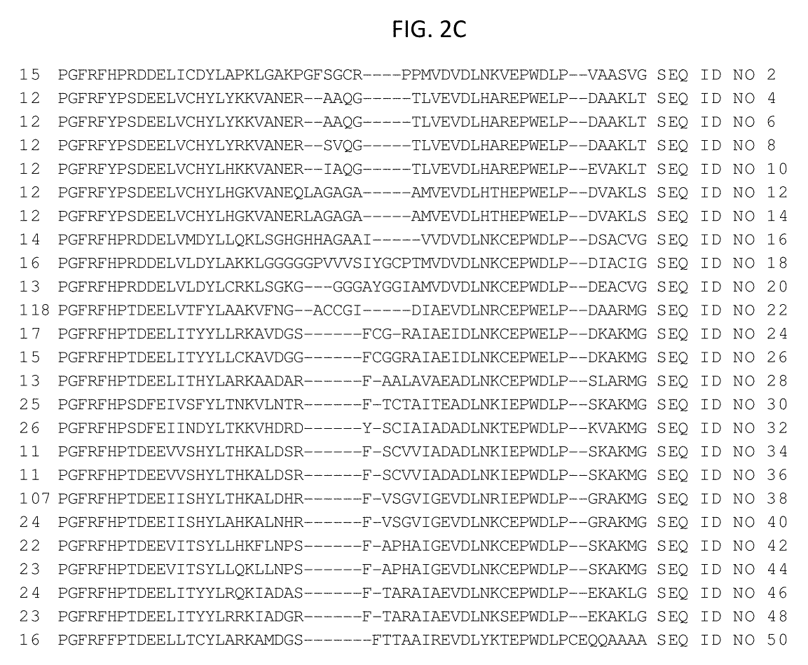

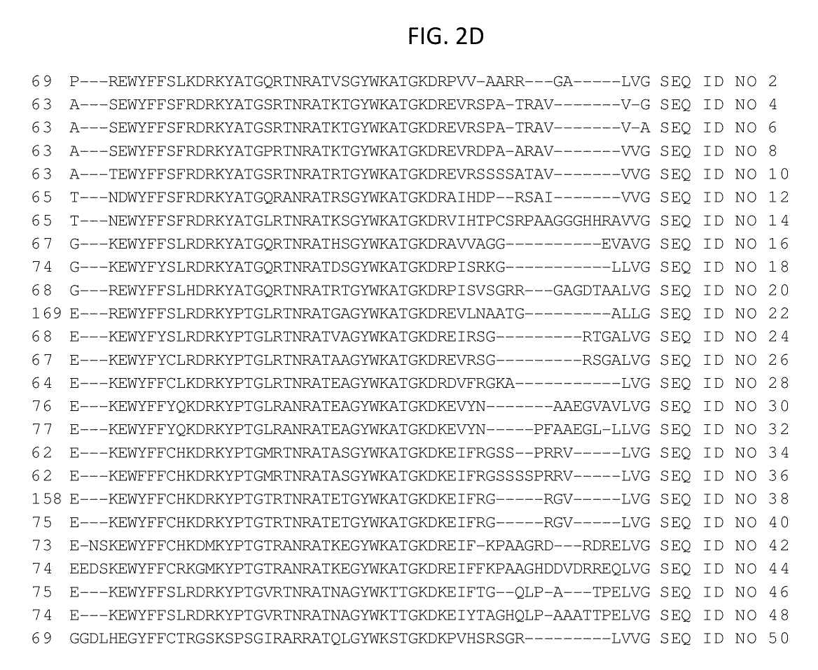

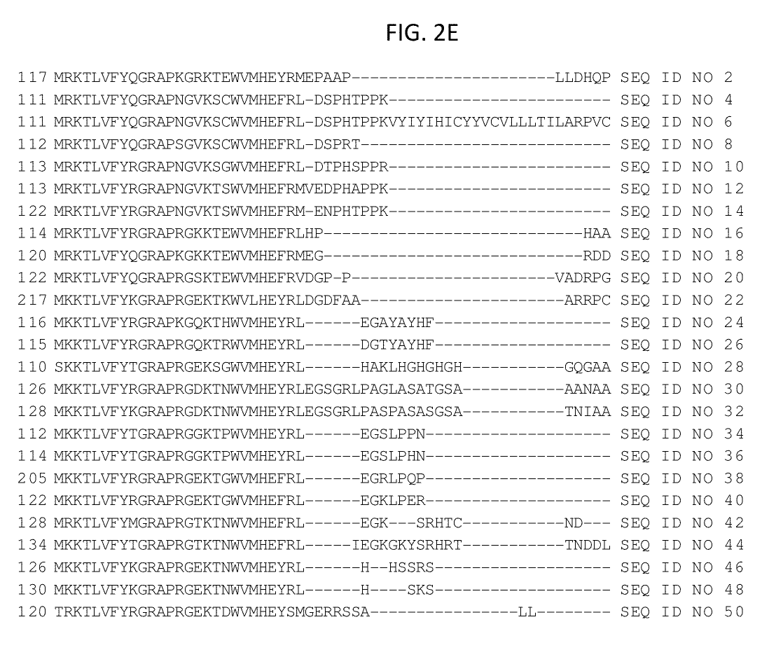

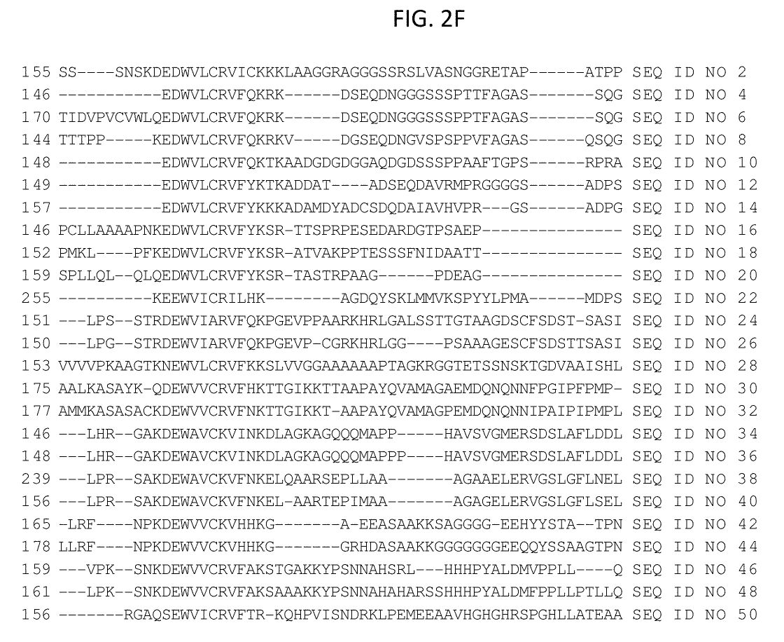

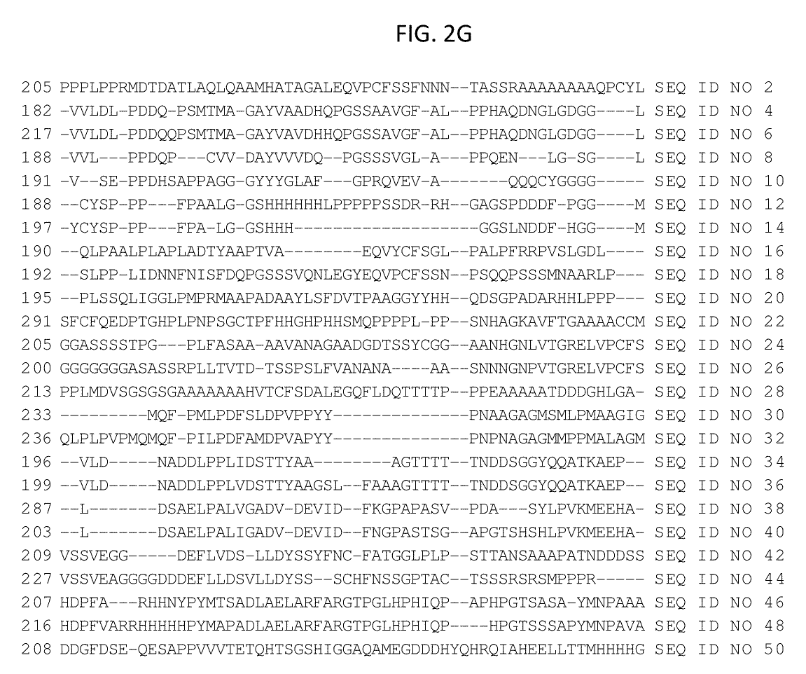

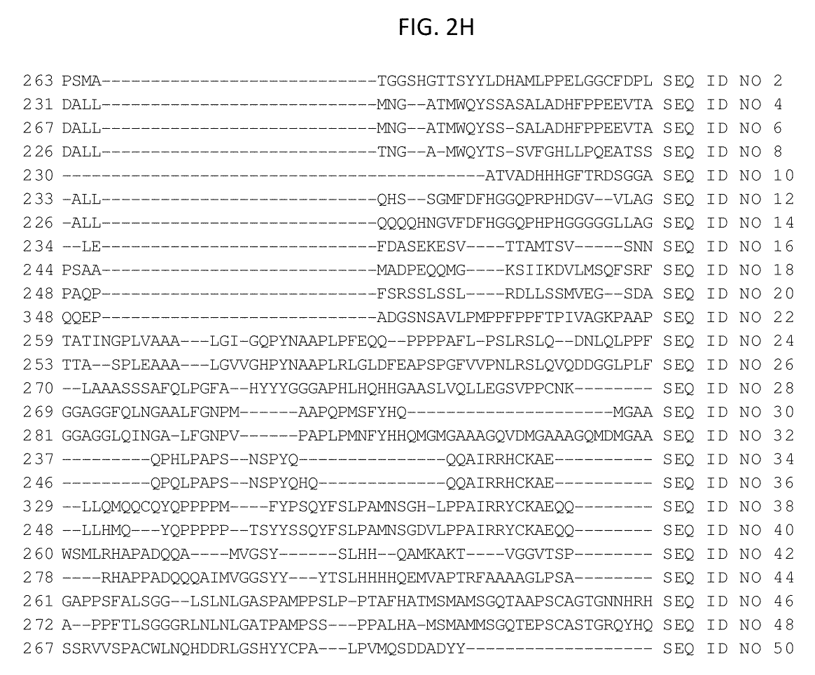

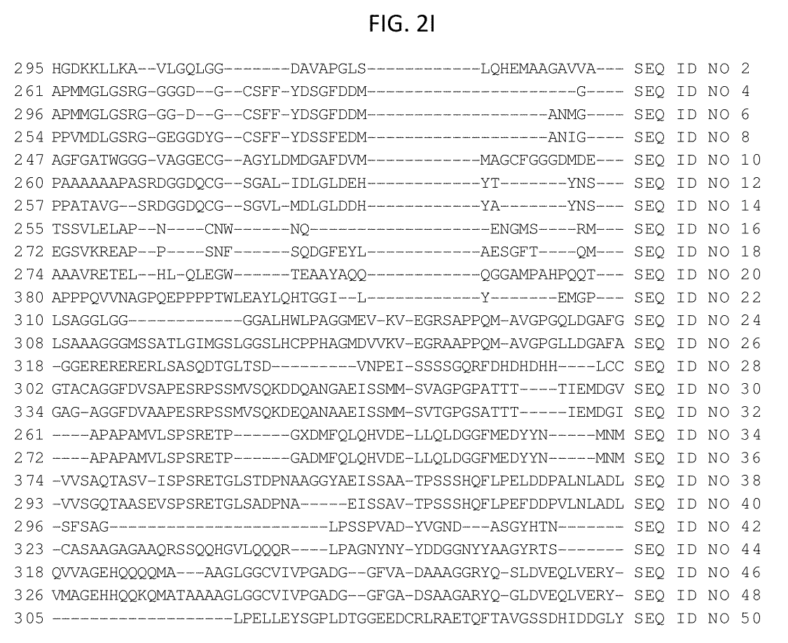

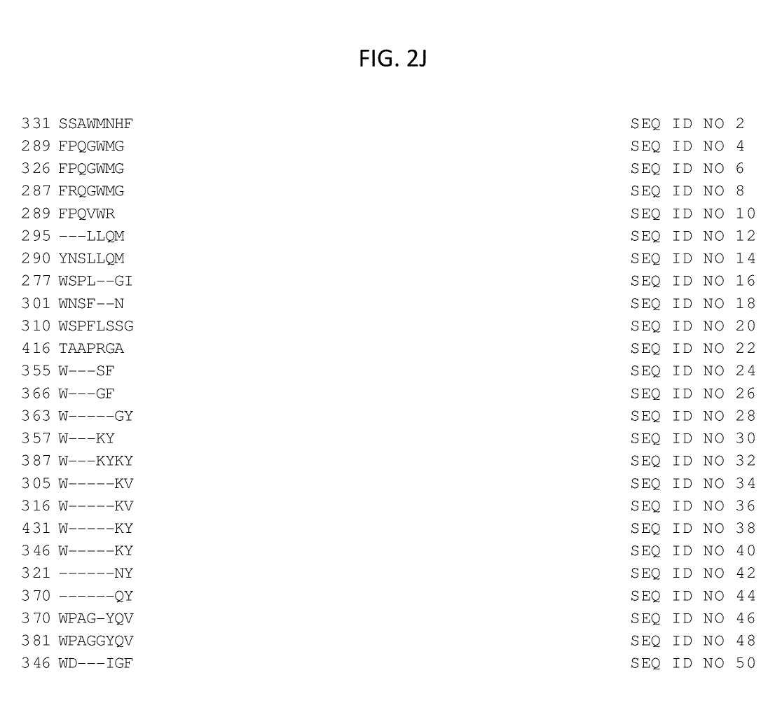

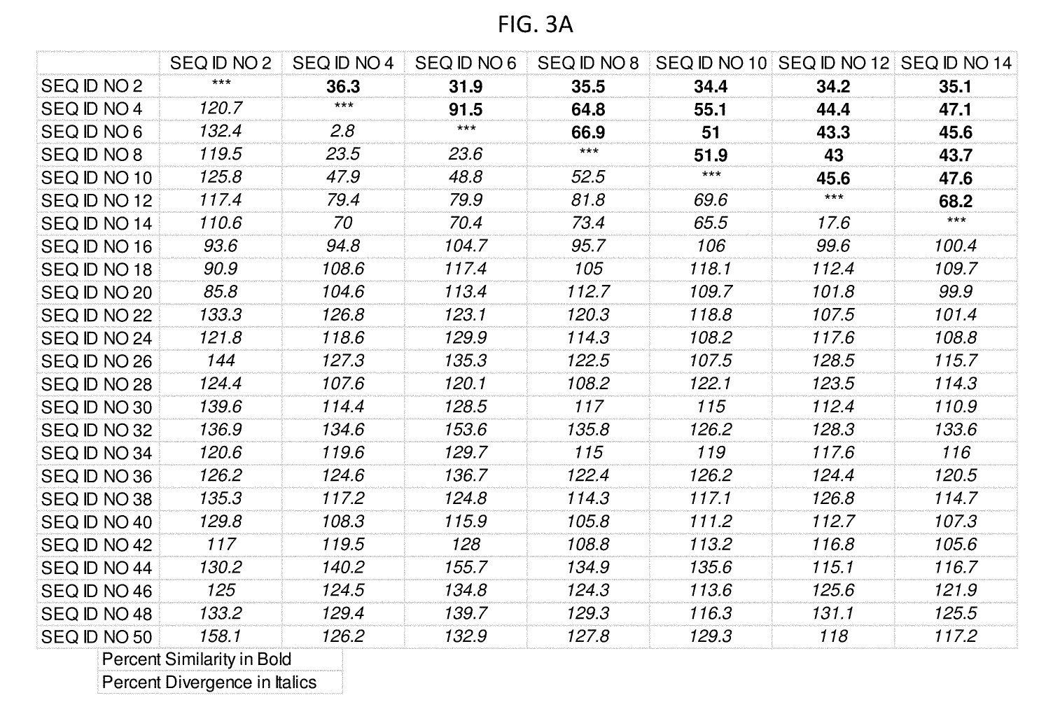

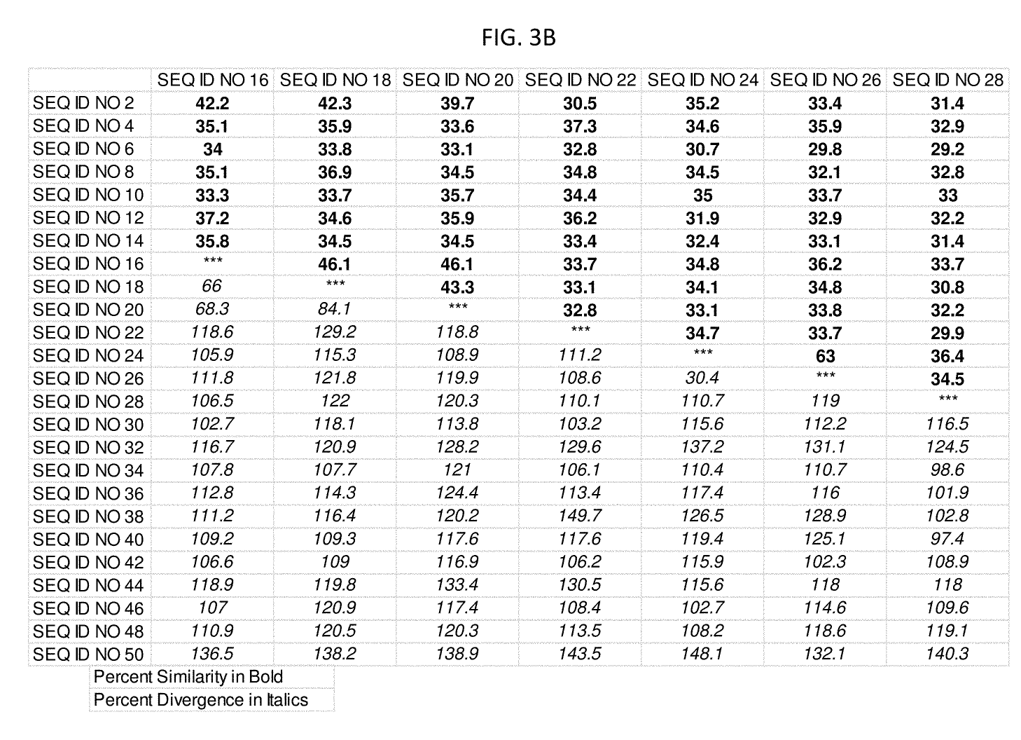

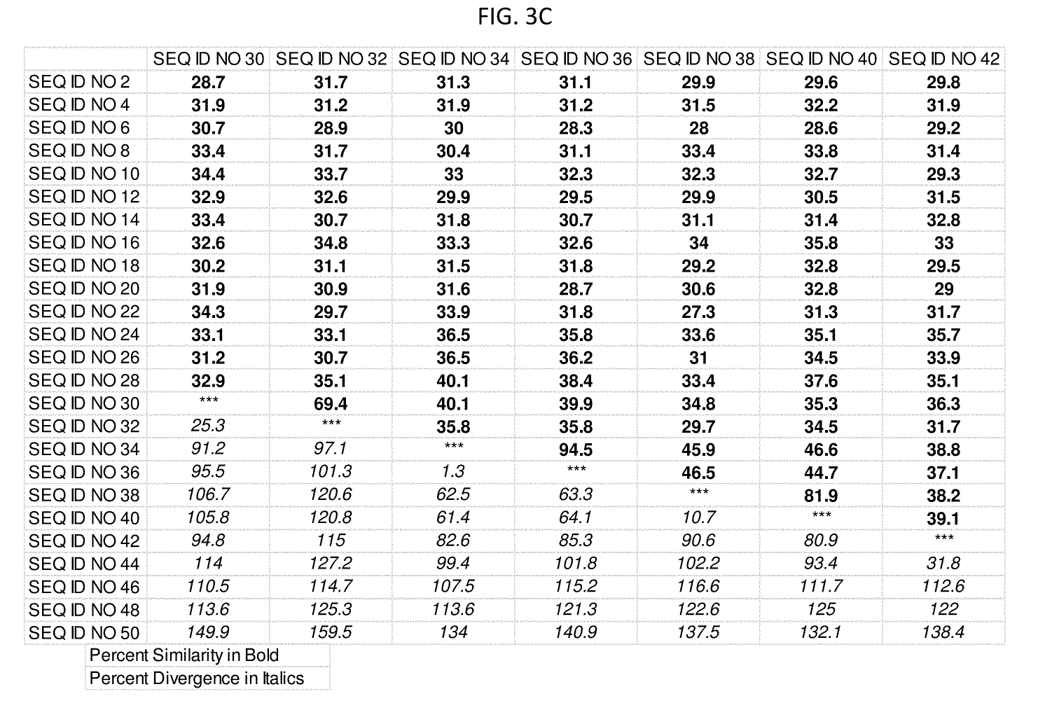

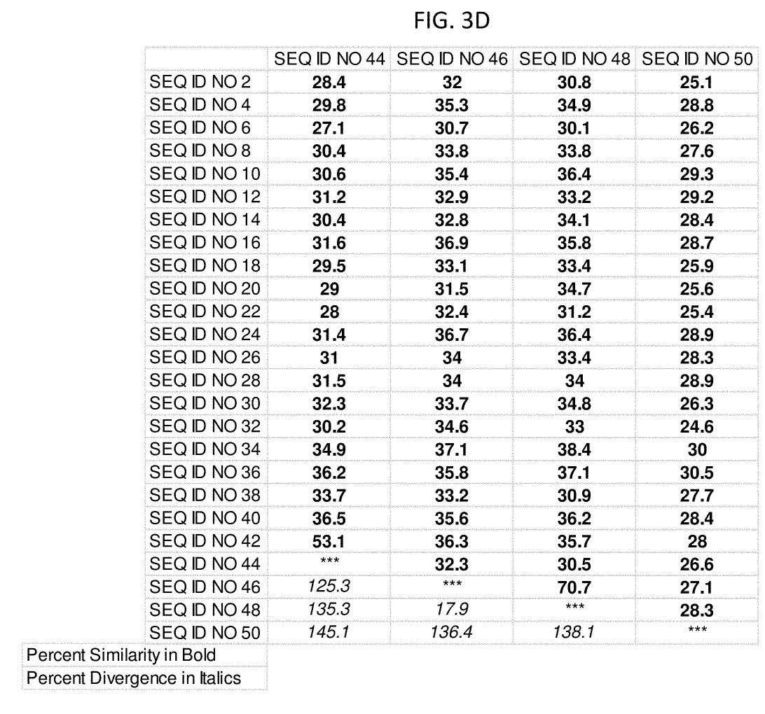

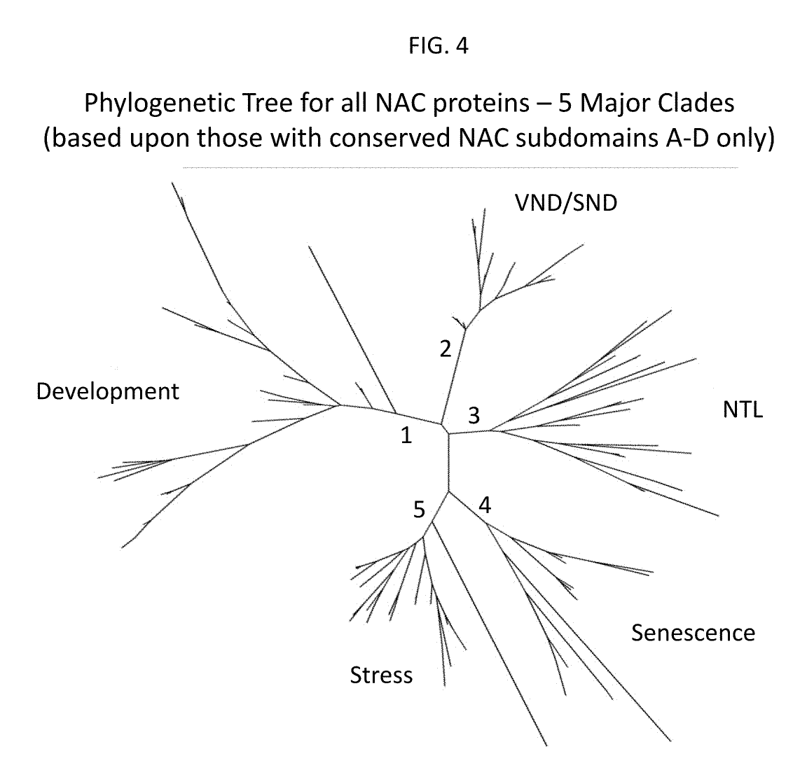

SEQ ID NO:56 is the consensus sequence obtained by aligning the maize YEP6 polypeptides from clade 1 (FIG. 4) shown in FIGS. 2A-2J (SEQ ID NOs:2, 4, 6, 8, 10, 12, 14, 16, 18, 20, 22, 24, 26, 28, 30, 32, 34, 36, 38, 40, 42, 44, 46, 48 and 50).

Table 2 lists the CDS sequences of YEP6 polypeptides from Rice and Sorghum (SEQ ID NOs:57-98)

TABLE 2
YEP6 Polypeptides from Rice and Sorghum

| | | SEQ ID NO: |
|---|---|---|
| Plant | YEP6 polypeptide | (Amino Acid) |
| Rice | LOC_Os12g03050.1 | 57 |
| Rice | LOC_Os12g41680.1 | 58 |
| Rice | LOC_Os09g32260.1 | 59 |
| Rice | LOC_Os08g40030.1 | 60 |
| Rice | LOC_Os08g10080.1 | 61 |
| Rice | LOC_Os04g38720.1 | 62 |
| Rice | LOC_Os03g42630.1 | 63 |
| Rice | LOC_Os03g21030.1 | 64 |
| Rice | LOC_Os02g36880.1 | 65 |
| Rice | LOC_Os11g03370.1 | 66 |
| Rice | LOC_Os11g04470.1 | 67 |
| Rice | LOC_Os12g04230.1 | 68 |
| Rice | LOC_Os01g01470.1 | 69 |
| Rice | LOC_Os01g29840.1 | 70 |
| Rice | LOC_Os02g06950.1 | 71 |
| Rice | LOC_Os06g46270.1 | 72 |
| Rice | LOC_Os09g024560.1 | 73 |
| Rice | LOC_Os01g01470.1 | 74 |
| Sorghum | Sb01g036590.1 | 75 |
| Sorghum | Sb01g043270.1 | 76 |
| Sorghum | Sb04g023990.1 | 77 |
| Sorghum | Sb06g019010.1 | 78 |
| Sorghum | Sb05g001590.1 | 79 |
| Sorghum | Sb02g023960.1 | 80 |
| Sorghum | Sb005g024550.1 | 81 |
| Sorghum | Sb03g008470.1 | 82 |
| Sorghum | Sb03g008860.1 | 83 |
| Sorghum | Sb07g027650.1 | 84 |
| Sorghum | Sb02g028870.1 | 85 |
| Sorghum | Sb08g006330.1 | 86 |
| Sorghum | Sb02g024530.1 | 87 |
| Sorghum | Sb07g021200.1 | 88 |
| Sorghum | Sb03g010130.1 | 89 |
| Sorghum | Sb02g032220.1 | 90 |
| Sorghum | Sb02g032230.1 | 91 |
| Sorghum | Sb10g027100.1 | 92 |
| Sorghum | Sb02g029460.1 | 93 |
| Sorghum | Sb07g005610.1 | 94 |
| Sorghum | Sb06g028800.1 | 95 |
| Sorghum | Sb04g36640.1 | 96 |
| Sorghum | Sb04g026440.1 | 97 |
| Sorghum | Sb01g014310.1 | 98 |

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The term “ZmYEP6 gene” refers herein to the gene that encodes for a ZmYEP6 polypeptide. A ZmYEP6 DNA sequence is given herein in SEQ ID NO:1. The term “ZmYEP6 polypeptide” refers herein to a Zea mays polypeptide that is represented by the amino acid sequence SEQ ID NO:2, or a polypeptide with at least 80% sequence identity to SEQ ID NO:2.

The disclosure also encompasses other Zea mays homologues of ZmYEP6 (see Table 1) that are clustered with it in clade 1 in the phylogenetic tree shown in FIG. 4.

The term “YEP6 polypeptide” refers herein to the polypeptide given in SEQ ID NO:2 and the homologs clustered with SEQ ID NO:2 in clade 1 (FIG. 4 and Tables 1 and 2). The term “YEP6 polypeptide” refers herein to the ZmYEP6 polypeptide and its homologs or orthologs from maize or other plant species. The terms OsYEP6, SbYEP6 and GmYEP6 refer respectively to YEP6 homologs from Oryza sativa, Sorghum bicolor and Glycine max.

The term “YEP6 polypeptide”, as referred to herein is a polypeptide comprising an amino acid sequence with at least 80% sequence identity to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98.

YEP6 polypeptides as referred to herein, belong to the NAC superfamily of transcription factors.

NAC (Petunia NAM, Arabidopsis ATAF1/2 and CUC2) proteins belong to a plant-specific transcription factor superfamily, whose members contain a conserved sequence known as the DNA-binding NAC-domain in the N-terminal region and a variable transcriptional regulatory C-terminal region. Based on its motif distribution, the NAC-domain can be divided into five sub-domains (A-E) (Zhu et al Evolution 66-6: 1833-1848; Ooka et al. (2003) DNA Research 10, 239-247). The C-terminal regions of some NAC TFs (transcription factors) also contain transmembrane motifs (TMs), which anchor to the plasma membrane. (Lu et al (2012) Plant Cell Rep 31:1701-1711; Tran et al. (2004) Plant Cell 16:2481-2498). At least 117 and 151 NAC family members have been predicted in Arabidopsis and rice, respectively (Nuruzzaman et al. (2010) Gene 465:30-44).

A phylogenetic tree showing classification of NAC proteins is shown in FIG. 4. YEP6 proteins belong to cladel, or the development clade. The YEP6 polypeptides described herein comprise the PF02365 or the NAM domain (Hu et al BMC Plant Biology 2010, 10:145).

NAC proteins have also been implicated in transcriptional control in a variety of plant processes, including in the development of the shoot apical meristem and floral organs, and in the formation of lateral roots. Arabidopsis NAC gene CUC3 has been reported to contribute to the establishment of the cotyledon boundary and the shoot meristem (Li et al. (2012) BMC Plant Biology, 12:220).

NAC proteins have also been implicated in responses to stress and viral infections (Ernst et al. (2004), EMBO Reports 5, 3, 297-303; Guo and Gan Plant Journal (2006) 46, 601-612, Yoon et al. Mol. Cells, Vol. 25, No. 3, pp. 438-445).

Overexpression of some NAC genes has been shown to significantly increase the drought and salt tolerance of a number of plants (Zheng et al. (2009) Biochem. Biophys. Res. Commun. 379:985-989; Lu et al (2012) Plant Cell Rep 31:1701-1711). Transgenic Arabidopsis plants overexpressing ZmSNAC1, a Zea mays NAC1 have been shown to exhibit enhanced sensitivity to ABA and osmotic stress in the germination stage, and exhibited increased tolerance to dehydration in the seedling stage. (Lu et al Plant Cell Rep (2012) 31:1701-1711).

Some NAC proteins have also been shown to be positive regulators of senescence initiation, such as the Arabidopsis NAC transcription factor, AtNAP, and the GPC protein in wheat (Uauy et al (2006) Science, 24 November, vol 314; Thomas and Ougham Journal of Experimental Botany, Vol. 65, No. 14, pp. 3889-3900, 2014; Lee et al Plant J. (2012) 70, 831-844; Guo and Gan (2006) Plant J. 46, 601-612.

Overexpression of some NAC family proteins, such as JUB1 in Arabidopsis thaliana has been shown to strongly delay senescence and enhance tolerance to various abiotic stresses (Wu et al (2012) Plant Cell, Vol. 24: 482-506.

Shiriga et al did a genome-wide analysis in maize identified 152 NAC TFs, while Zhu et al have predicted about 117 NAC proteins in maize (Shiriga et al Metagene 2(2014) 407-417, Zhu et al Evolution 66-6: 1833-1848).

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Nutrients include, but are not limited to, the following: nitrogen (N), phosphorus (P), potassium (K), calcium (Ca), magnesium (Mg) and sulfur (S). For example, the abiotic stress may be drought stress, low nitrogen stress, or both.

“Nitrogen limiting conditions” or “low nitrogen stress” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Nitrogen stress tolerance” is a trait of a plant and refers to the ability of the plant to survive under nitrogen limiting conditions over prolonged periods of time, without exhibiting the same degree of physiological or physical deterioration relative to the reference or control plant grown under similar stress conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

“Environmental conditions” refer to conditions under which the plant is grown, such as the availability of water, availability of nutrients (for example nitrogen), or the presence of insects or disease.

“Stay-green” or “staygreen” is a term used to describe a plant phenotype, e.g., whereby leaf senescence (most easily distinguished by yellowing of leaf associated with chlorophyll degradation) is delayed compared to a standard reference or a control. The staygreen phenotype has been used as selective criterion for the development of improved varieties of crop plants such as corn, rice and sorghum, particularly with regard to the development of stress tolerance, and yield enhancement (Borrell et al. (2000b) Crop Sci. 40:1037-1048; Spano et al, (2003) J. Exp. Bot. 54:1415-1420; Christopher et al, (2008) Aust. J. Agric. Res. 59:354-364, 2008, Kashiwagi et al (2006) Plant Physiology and Biochemistry 44:152-157, 2006 and Zheng et al, (2009) Plant Breed 725:54-62.

“Increase in staygreen phenotype” as referred to in here, indicates retention of green leaves, delayed foliar senescence and significantly healthier canopy in a plant, compared to control plant.

Staygreen plants have been categorized broadly into “cosmetic staygreen” and “functional staygreen”. In plants exhibiting cosmetic staygreen phenotype, the primary lesion of senescence is confined to pigment catabolism. In plants exhibiting functional staygreen phenotype the entire senescence syndrome, of which chlorophyll catabolism is only one component, is delayed or slowed down, or both. The functional staygreen trait has been shown to be associated with the transition from the carbon (C) capture to the nitrogen (N) mobilization phase of foliar development (Thomas and Oughan (2014) J Exp Bot. Vol. 65 (14), pp. 3889-3900; Kusaba et al (2013) Photosynth Res 117:221-234; Thomas and Howarth (2000) J Exp Bot. Vol. 51, pp. 329-337

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America) Special Publication No. 29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

“Transgenic” generally refers to any cell, cell line, callus, tissue, plant part or plant, the genome of which has been altered by the presence of a heterologous nucleic acid, such as a suppression DNA construct or a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a suppression DNA construct or a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. In case of suppression DNA constructs, as disclosed herein, gene stacking approach may encompass silencing of more than one YEP6 gene, or may also refer to stacking of a suppression DNA construct with a recombinant DNA construct that leads to overexpression of a particular gene or polypeptide. Gene stacking can be accomplished by many means including but not limited to co-transformation, retransformation, and crossing lines with different transgenes.

The suppression DNA constructs and nucleic acid sequences of the current disclosure may be used in combination (“stacked”) with other polynucleotide sequences of interest in order to create plants with a desired phenotype. The desired combination may affect one or more traits; that is, certain combinations may be created for modulation of gene expression affecting YEP6 gene activity or expression. Other combinations may be designed to produce plants with a variety of desired traits including but not limited to increased yield and altered agronomic characteristics. “Transgenic plant” also includes reference to plants which comprise more than one heterologous polynucleotide within their genome. Each heterologous polynucleotide may confer a different trait to the transgenic plant.

The term “endogenous” relates to any gene or nucleic acid sequence that is already present in a cell.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” generally refers to the RNA that is without introns and that can be translated into protein by the cell.

“cDNA” generally refers to a DNA that is complementary to and synthesized from an mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein generally refers to a post-translationally processed polypeptide; i.e., one from which any pre- or pro-peptides present in the primary translation product have been removed.

“Precursor” protein generally refers to the primary product of translation of mRNA; i.e., with pre- and pro-peptides still present. Pre- and pro-peptides may be and are not limited to intracellular localization signals.

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“Suppression DNA construct” is a recombinant DNA construct which when transformed or stably integrated into the genome of the plant, results in “silencing” of a target gene in the plant. Examples of such suppression DNA constructs include, but are not limited to, cosuppression constructs, antisense constructs, viral suppression constructs, hairpin suppression constructs, stem-loop suppression constructs, double-stranded RNA-producing constructs, RNA silencing constructs, RNA interference constructs, and ribozyme constructs.

The current disclosure provides for plants that have a disruption/mutation in at least one endogenous YEP6 gene, that leads to silencing or reduction in expression or activity of the at least one YEP6 polypeptide, in at least one tissue in at least one developmental stage, compared to a control plant that does not have any silencing or reduction in the YEP6 gene expression or YEP6 polypeptide activity, and lacks the disruption/mutation in the YEP6 gene.

In one aspect, the at least one YEP6 polypeptide comprises two or more YEP6 polypeptides. In one aspect, the at least one YEP6 polypeptide comprises three or more YEP6 polypeptides.

The terms “reference”, “reference plant”, “control”, “control plant”, “wild-type” or “wild-type plant” are used interchangeably herein, and refers to a parent, null, or non-transgenic plant of the same species that lacks the disruption/mutation or silencing of the YEP6 gene. A control plant as defined herein is a plant that is not made according to any of the methods disclosed herein. A control plant can also be a parent plant that contains a wild-type allele of a YEP6 gene. A wild-type plant would be: (1) a plant that carries the unaltered or not modulated form of a gene or allele, or (2) the starting material/plant from which the plants produced by the methods described herein are derived.

“Silencing,” as used herein with respect to the target gene, refers generally to the reduction or inhibition of levels of mRNA or protein/enzyme expressed by the target gene, and/or the level of the enzyme activity or protein functionality.

The terms “reduction”, “downregulation”, “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, small RNA-based approaches, or genome disruption approaches.

Many techniques can be used for producing a plant having a disruption in at least one YEP6 gene, where the disruption results in a reduced expression or activity of the YEP6 polypeptide encoded by the YEP6 gene compared to a control plant. The disruption can be a result of introducing a suppression DNA construct that is effective for inhibiting the expression of the YEP6 gene, or for mutagenizing the YEP6 gene.

Down regulation of expression or activity of the YEP6 gene or polypeptide is by at least 10%, 20%, 30%, 40%, 50%, 60%, 70%, 80%, 90% or even complete (100%) loss of activity or expression.

Various assays for measuring gene expression are well known in the art and can be done at the protein level (examples include, but are not limited to, Western blot, ELISA) or at the mRNA level such as by RT-PCR.

In certain aspects of the disclosure, the suppression DNA construct is sense or antisense suppression DNA construct.

One method of reducing the expression of a YEP6 gene is by sense suppression/cosuppression. Introduction of expression cassettes in which a nucleic acid is configured in the sense orientation with respect to the promoter has been shown to be an effective means by which to block the transcription of the corresponding target gene. For example Napoli et al (1990) Plant Cell 2:279-289, and U.S. Pat. Nos. 5,034,323; 5,231,0202 and 5,283,184.

Cosuppression constructs in plants have been previously designed by focusing on overexpression of a nucleic acid sequence corresponding to all or part of a native mRNA, in the sense orientation, which results in the reduction of all RNA having homology to the overexpressed sequence (see Vaucheret et al., Plant J. 16:651-659 (1998); and Gura, Nature 404:804-808 (2000)).

The polynucleotide used for cosuppression may correspond to all or part of the sequence encoding the target gene, and cosuppression constructs may contain sequences from coding regions or non-coding regions, e.g., introns, 5′-UTRs and 3′-UTRs, or both.

Methods for using cosuppression to inhibit the expression of endogenous genes in plants are described in Flavell, et al., (1995) Proc. Natl. Acad. Sci. USA 91:3590-3596; Jorgensen, et al. (1996) Plant Mol. Biol. 31:957-973; Johansen and Carrington, (2001) Plant Physiol. 126:930-938; Broin, et al., (2002) Plant Cell 15:1517-1532; Stoutjesdijk, et al., (2002) Plant Physiol. 129:1723-1731; Yu, et al., (2003) Phytochemistry 63:753-763; and U.S. Pat. Nos. 5,035,323, 5,283,185 and 5,952,657.

“Antisense inhibition” refers to the production of antisense RNA transcripts capable of suppressing the expression of the target gene or gene product. “Antisense RNA” refers to an RNA transcript that is complementary to all or part of a target nucleic acid and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence. A duplex can form between the antisense sequence and its complementary sense sequence, resulting in reducing or inhibiting expression from the gene (U.S. Pat. No. 7,763,773).

Use of antisense nucleic acids is well known in the art (U.S. Pat. No. 5,759,829, U.S. Pat. No. 6,242,258, U.S. Pat. No. 6,500,615 and U.S. Pat. No. 5,942,657). An antisense nucleic acid can be produced by a number of well-established techniques, examples include, but are not limited to, chemical synthesis of an antisense RNA or oligonucleotide of at least about 15 bases and complementary to unique regions of the mRNA transcript sequence encoding a YEP6 polypeptide (a homolog or a derivative thereof can be synthesized, e.g., by conventional phosphodiester techniques), or in vitro transcription.

Another variation describes the use of plant viral sequences to direct the suppression of proximal mRNA encoding sequences (PCT Publication No. WO 98/36083 published on Aug. 20, 1998).

Another method of reducing YEP6 gene expression is by RNA interference (RNAi) or RNA silencing.

The terms “RNA interference” or “RNAi” as used herein refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. As used herein, RNAi refers to a mechanism through which presence of a double-stranded RNA in a cell results in reduction in expression of the corresponding target gene, for example, expression of a hairpin (stem-loop) RNA or of the two strands of an interfering RNA will lead to silencing of a target gene by RNA interference.

The process of RNA interference is well described in the literature, as are methods for determining appropriate interfering RNA(s) to target a desired gene, e.g., a YEP6 gene, and for generating such interfering RNAs. For example, RNA interference is described in (US patent publications US20020173478, US20020162126, and US20020182223) “RNA interference” Nature., July 11; 418(6894):244-51; Ueda R. (2001) “RNAi: a new technology in the postgenomic sequencing era” J Neurogenet; 15(3-4):193-204; Ullu et al (2002) “RNA interference: advances and questions” Philos Trans R Soc Lond B Biol Sci. January 29; 357(1417): 65-70; Fire et al., Trends Genet. 15:358 (1999); U.S. Pat. No. 7,763,773)

In one aspect, a suppression DNA construct is introduced into a plant to silence one or more YEP6 genes, by RNA interference or RNAi. For example, a sequence or subsequence includes a small subsequence, e.g., about 21-25 bases in length (with, e.g., at least 80%, at least 90%, or 100% identity to one or more YEP6 gene subsequences), a larger subsequence, e.g., 25-100 or 100-2000 (or 200-1500, 250-1000 etc.) bases in length (with at least one region of about 21-25 bases of at least 80%, at least 90%, or 100% identity to one or more YEP6 gene subsequences) and/or the entire coding sequence or gene.

In one embodiment of the current disclosure, RNA interference is used to inhibit or reduce the expression of a YEP6 gene in a transgenic plant.

The YEP6 polynucleotide sequence or subsequence to be expressed to induce RNAi can be expressed under control of any promoter, examples for which are, but are not limited to, constitutive promoter, inducible promoter or a tissue-specific promoter.

A polynucleotide sequence is said to “encode” a sense or antisense RNA molecule, or RNA silencing or interference molecule or a polypeptide, if the polynucleotide sequence can be transcribed (in spliced or unspliced form) and/or translated into the RNA or polypeptide, or a subsequence thereof.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as might be indicated by the context.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Catalytic RNA molecules or ribozymes can also be used to inhibit expression of YEP6 genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them thereby increasing the activity of the constructs.

A number of classes of ribozymes have been identified. The design and use offtarget RNA-specific ribozymes has been described (Haseloff et al. (1988) Nature, 334:585-591, U.S. Pat. No. 5,987,071, PCT Publication No. WO2013/065046).

Gene Disruption Techniques:

The expression or activity of the YEP6 gene and/or polypeptide can be reduced by disrupting the gene encoding the YEP6 polypeptide. The YEP6 gene can be disrupted by any means known in the art. One way of disrupting a gene is by insertional mutagenesis. The gene can be disrupted by mutagenizing the plant or plant cell using random or targeted mutagenesis.

The YEP6 gene can be disrupted by transposon tagging, also known as transposon based gene inactivation. In one embodiment, the inactivating step comprises producing one or more mutations in a YEP6 gene sequence, where the one or more mutations in the YEP6 gene sequence comprise one or more transposon insertions, thereby inactivating the YEP6 gene, compared to a corresponding control plant.

A “transposable element” (TE) or “transposable genetic element” is a DNA sequence that can move from one location to another in a cell.

Transposable elements can be categorized into two broad classes based on their mode of transposition. These are designated Class I and Class II; both have applications as mutagens and as delivery vectors. Class I transposable elements transpose by an RNA intermediate and use reverse transcriptases, i.e., they are retroelements. There are at least three types of Class I transposable elements, e.g., retrotransposons, retroposons, SINE-like elements. Retrotransposons typically contain LTRs, and genes encoding viral coat proteins (gag) and reverse transcriptase, RnaseH, integrase and polymerase (pol) genes. Numerous retrotransposons have been described in plant species. Such retrotransposons mobilize and translocate via a RNA intermediate in a reaction catalyzed by reverse transcriptase and RNase H encoded by the transposon. Examples fall into the Tyl-copia and Ty3-gypsy groups as well as into the SINE-like and LINE-like classifications (Kumar and Bennetzen (1999) Annual Review of Genetics 33:479). In addition, DNA transposable elements such as Ac, Taml and En/Spm are also found in a wide variety of plant species, and can be utilized in the methods disclosed herein. Transposons (and IS elements) are common tools for introducing mutations in plant cells.

Other mutagenic methods can also be employed to introduce mutations in the YEP6 gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

“TILLING” or “Targeting Induced Local Lesions IN Genomics” refers to a mutagenesis technology useful to generate and/or identify, and to eventually isolate mutagenized variants of a particular nucleic acid with modulated expression and/or activity (McCallum et al., (2000), Plant Physiology 123:439-442; McCallum et al., (2000) Nature Biotechnology 18:455-457; and, Colbert et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter for example). These mutant variants may even exhibit lower YEP6 activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (U.S. Pat. No. 8,071,840).

Other detection methods for detecting mutations in the YEP6 gene can be employed, e.g., capillary electrophoresis (e.g., constant denaturant capillary electrophoresis and single-stranded conformational polymorphism). In another example, heteroduplexes can be detected by using mismatch repair enzymology (e.g., CELI endonuclease from celery). CELI recognizes a mismatch and cleaves exactly at the 3′ side of the mismatch. The precise base position of the mismatch can be determined by cutting with the mismatch repair enzyme followed by, e.g., denaturing gel electrophoresis. See, e.g., Oleykowski et al., (1998) “Mutation detection using a novel plant endonuclease” Nucleic Acid Res. 26:4597-4602; and, Colbert et al., (2001) “High-Throughput Screening for Induced Point Mutations” Plant Physiology 126:480-484.

The plant containing the mutated YEP6 gene can be crossed with other plants to introduce the mutation into another plant. This can be done using standard breeding techniques.

Homologous recombination allows introduction in a genome of a selected nucleic acid at a defined selected position. Homologous recombination has been demonstrated in plants. See, e.g., Puchta et al. (1994), Experientia 50: 277-284; Swoboda et al. (1994), EMBO J. 13: 484-489; Offringa et al. (1993), Proc. Natl. Acad. Sci. USA 90: 7346-7350; Kempin et al. (1997) Nature 389:802-803; and, Terada et al., (2002) Nature Biotechnology, 20(10):1030-1034).

Methods for performing homologous recombination in plants have been described not only for model plants (Offringa et al. (1990) EMBO J. October; 9(10):3077-84) but also for crop plants, for example rice (Terada R, Urawa H, Inagaki Y, Tsugane K, lida S. Nat Biotechnol. 2002 20(10):1030-4; lida and Terada: Curr Opin Biotechnol. 2004 April; 15(2):1328). The nucleic acid to be introduced (which may be YEP6 nucleic acid or a variant thereof) need not be targeted to the locus of the YEP6 gene, but may be introduced into, for example, regions of high expression. The nucleic acid to be introduced may be a dominant negative allele used to replace the endogenous gene or may be introduced in addition to the endogenous gene.

The present disclosure encompasses variants and subsequences of the polynucleotides and polypeptides described herein.

The term “variant” with respect to a polynucleotide or DNA refers to a polynucleotide that contains changes in which one or more nucleotides of the original sequence is deleted, added, and/or substituted while substantially maintaining the function of the polynucleotide. For example, a variant of a promoter that is disclosed herein can have minor changes in its sequence without substantial alteration to its regulatory function.

The term “variant” with respect to a polypeptide refers to an amino acid sequence that is altered by one or more amino acids with respect to a reference sequence. The variant can have “conservative changes, wherein a substituted amino acid has similar structural or chemical properties, for example, and replacement of leucine with isoleucine. Alternatively, a variant can have “non-conservative” changes, for example, replacement of a glycine with a tryptophan. Analogous minor variation can also include amino acid deletion or insertion, or both.

Guidance in determining which nucleotides or amino acids for generating polynucleotide or polypeptide variants can be found using computer programs well known in the art.

The terms “fragment” and “subsequence” are used interchangeably herein, and refer to any portion of an entire sequence.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Allelic variants encompass Single nucleotide polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

Plant breeding techniques known in the art and used in the maize plant breeding program include, but are not limited to, recurrent selection, bulk selection, mass selection, backcrossing, pedigree breeding, open pollination breeding, restriction fragment length polymorphism enhanced selection, genetic marker enhanced selection, double haploids and transformation. Often combinations of these techniques are used.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=1 0, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Complete sequences and figures for vectors described herein (e.g., pHSbarENDs2, pDONR™/Zeo, pDONR™221, pBC-yellow, PHP27840, PHP23236, PHP10523, PHP23235 and PHP28647) are given in PCT Publication No. WO/2012/058528, the contents of which are herein incorporated by reference.

### Turning Now to the Embodiments

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

In one embodiment, a plant in which expression of an endogenous YEP6 gene is reduced, when compared to a control plant, wherein the YEP6 gene encodes a YEP6 polypeptide and wherein the plant exhibits at least one phenotype selected from the group consisting of: increased yield, increased abiotic stress tolerance, increased staygreen, and increased biomass compared to the control plant.

In one embodiment, a plant in which activity of an endogenous YEP6 polypeptide is reduced, when compared to the activity of wild-type YEP6 polypeptide in a control plant, wherein the plant exhibits at least one phenotype selected from the group consisting of: increased yield, increased abiotic stress tolerance, increased staygreen, and increased biomass compared to the control plant.

In one embodiment, the plant exhibits increased abiotic stress tolerance, and the abiotic stress is drought stress, low nitrogen stress, or both. In one embodiment, the plant exhibits the phenotype of increased yield and the phenotype is exhibited under non-stress conditions. In one embodiment, the plant exhibits the phenotype of increased yield and the phenotype is exhibited under stress conditions. In one embodiment, the plant exhibits the phenotype under drought stress conditions.

In one embodiment, the endogenous YEP6 polypeptide comprises an amino acid sequence with at least 80% sequence identity to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98.

In one embodiment, the plant is a monocot plant. In another embodiment, the plant is a maize plant.

In one embodiment, the reduction in expression of the endogenous YEP6 gene is caused by sense suppression, antisense suppression, miRNA suppression, ribozymes, or RNA interference. In one embodiment, the reduction in expression of the endogenous YEP6 gene is caused by a mutation in the endogenous YEP6 gene. In one embodiment, the mutation in the endogenous YEP6 gene is caused by insertional mutagenesis. In one embodiment, the insertional mutagenesis is caused by transposon mutagenesis.

One embodiment is a suppression DNA construct comprising a polynucleotide, wherein the polynucleotide is operably linked to a heterologous promoter in sense or antisense orientation, or both, wherein the construct is effective for reducing expression of an endogenous YEP6 gene in a plant, and wherein the polynucleotide comprises: (a) the nucleotide sequence of SEQ ID NO:1, 3, 5, 7, 9, 11, 13, 15, 17, 19, 21, 23, 25, 27, 29, 31, 33, 35, 37, 39, 41, 43, 45, 47 or 49; (b) a nucleotide sequence that has at least 80% sequence identity, when compared to SEQ ID NO:1, 3, 5, 7, 9, 11, 13, 15, 17, 19, 21, 23, 25, 27, 29, 31, 33, 35, 37, 39, 41, 43, 45, 47 or 49; (c) a nucleotide sequence of at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49; (d) a nucleotide sequence that can hybridize under stringent conditions with the nucleotide sequence of (a); or (e) a modified plant miRNA precursor, wherein the precursor has been modified to replace the miRNA encoding region with a sequence designed to produce a miRNA directed to SEQ ID NO:1, 3, 5, 7, 9, 11, 13, 15, 17, 19, 21, 23, 25, 27, 29, 31, 33, 35, 37, 39, 41, 43, 45, 47 or 49.

One embodiment of the current disclosure encompasses the suppression DNA construct, wherein the polynucleotide comprises at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49, and the suppression DNA construct is designed for RNA interference, and is effective for reducing expression of YEP6 gene in a plant. In one embodiment, the polynucleotide comprises a nucleotide sequence that has at least 90% sequence identity to SEQ ID NO:55.

In one embodiment, the activity of the endogenous YEP6 polypeptide is reduced as a result of mutation of the endogenous YEP6 gene. In one embodiment, the mutation in the endogenous YEP6 gene is detected using the TILLING method.

One embodiment is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of introducing into a plant a suppression DNA construct comprising a polynucleotide operably linked to a heterologous promoter, wherein the suppression DNA construct is effective for reducing expression of an endogenous YEP6 gene. In one embodiment, the suppression DNA construct is selected from the group consisting of: sense suppression construct, antisense suppression construct, ribozyme construct, RNA interference construct and an miRNA construct. In one embodiment, the suppression DNA construct is an RNA interference construct and the RNA interference construct comprises at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49, and wherein the RNA interference construct is effective for reducing the expression of the endogenous YEP6 gene. In one embodiment, the RNA interference construct comprises a polynucleotide sequence that has at least 90% sequence identity to SEQ ID NO:55.

One embodiment is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of: (a) introducing a mutation into an endogenous YEP6 gene; and (b) detecting said mutation using the Targeted Induced Local Lesions In Genomics (TILLING) method, wherein said mutation results in reducing expression of the endogenous YEP6 gene.

In one embodiment, the current disclosure includes a method of enhancing seed yield in a plant, when compared to a control plant, wherein the plant exhibits enhanced yield under either stress conditions, or non-stress conditions, or both, the method comprising the step of reducing expression of the endogenous YEP6 gene in a plant.

One embodiment of the current disclosure is a method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the step of utilizing a transposon to introduce an insertion into an endogenous YEP6 gene in a plant, wherein the insertion is effective for reducing expression of an endogenous YEP6 gene.

One embodiment of the current disclosure is a method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing into a plant a suppression DNA construct comprising a polynucleotide operably linked to a heterologous promoter, wherein the polynucleotide encodes a fragment or a variant of a polypeptide having an amino acid sequence of at least 80% sequence identity, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the fragment or the variant confers a dominant-negative phenotype in the plant.

In one embodiment, a method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing a mutation in an endogenous YEP6 gene, wherein the mutation is effective for reducing the activity of the endogenous YEP6 polypeptide. In one embodiment, the method further comprises the step of detecting the mutation and the detection is done using the Targeted Induced Local Lesions IN Genomics (TILLING) method.

The current disclosure also includes the plant obtained by any of the methods disclosed herein, wherein the plant exhibits at least one phenotype selected from the group consisting of: increased yield, increased staygreen, increased abiotic stress tolerance and increased biomass, compared to the control plant.

One embodiment of the current disclosure includes the plant comprising any of the suppression DNA constructs disclosed herein, wherein expression or activity of the endogenous YEP6 gene is reduced in the plant, when compared to a control plant, and wherein the plant exhibits at least one phenotype selected from the group consisting of: increased yield, increased staygreen, increased abiotic stress tolerance and increased biomass, compared to the control plant. In one embodiment, the plant exhibits an increase in abiotic stress tolerance, and the abiotic stress is drought stress, low nitrogen stress, or both. In one embodiment, the plant exhibits the phenotype of increased yield and the phenotype is exhibited under non-stress conditions. In one embodiment, the phenotype is exhibited under stress conditions.

In one embodiment, the plant is a monocot plant. In another embodiment, the monocot plant is a maize plant.

One embodiment of the current disclosure is a method of identifying one or more alleles associated with increased yield in a population of maize plants, the method comprising the steps of: (a) detecting in a population of maize plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, or a sequence that is 90% identical to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the one or more polymorphisms in the genomic region encoding the polypeptide or in the regulatory region controlling expression of the polypeptide is associated with yield; and (b) identifying one or more alleles at the one or more polymorphisms that are associated with increased yield.

In one embodiment, the one or more polymorphisms is in the coding region of the polynucleotide. In one embodiment, the regulatory region is a promoter element.

One embodiment encompasses the plants obtained by any of the methods disclosed herein, or comprising any of the suppression DNA constructs disclosed herein. The current disclosure also encompasses any progeny, or seeds obtained from the plants disclosed herein.

Isolated Polynucleotides and Polypeptides:

The present disclosure includes the following isolated polynucleotides and polypeptides:

An isolated polynucleotide comprising: (i) a nucleic acid sequence encoding a YEP6 polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides or a fragment or subsequence of the isolated polynucleotides may be utilized in any suppression DNA constructs of the present disclosure.

An isolated polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, and combinations thereof. The polypeptide is preferably a YEP6 polypeptide.

An isolated polynucleotide comprising (i) a nucleic acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9, 11, 13, 15, 17, 19, 21, 23, 25, 27, 29, 31, 33, 35, 37, 39, 41, 43, 45, 47 or 49, and combinations thereof; (ii) a full complement of the nucleic acid sequence of (i); or (iii) a fragment or subsequence of the nucleic acid sequence of (i). Any of the foregoing isolated polynucleotides or a fragment of the isolated polynucleotides may be utilized in any suppression DNA construct of the present disclosure. The isolated polynucleotide preferably encodes a YEP6 polypeptide.

An isolated polynucleotide comprising a nucleotide sequence, wherein the nucleotide sequence is hybridizable under stringent conditions with a DNA molecule comprising the full complement of SEQ ID NO:1, 3, 5, 7, 9, 11, 13, 15, 17, 19, 21, 23, 25, 27, 29, 31, 33, 35, 37, 39, 41, 43, 45, 47 or 49, or a subsequence thereof. The isolated polynucleotide preferably encodes a YEP6 polypeptide.

An isolated polynucleotide comprising a nucleotide sequence, wherein the nucleotide sequence is derived from SEQ ID NO:1, 3, 5, 7, 9, 11, 13, 15, 17, 19, 21, 23, 25, 27, 29, 31, 33, 35, 37, 39, 41, 43, 45, 47 or 49 by alteration of one or more nucleotides by at least one method selected from the group consisting of: deletion, substitution, addition and insertion. The isolated polynucleotide preferably encodes a YEP6 polypeptide. An isolated polynucleotide comprising a nucleotide sequence, wherein the nucleotide sequence corresponds to an allele of SEQ ID NO:1, 3, 5, 7, 9, 11, 13, 15, 17, 19, 21, 23, 25, 27, 29, 31, 33, 35, 37, 39, 41, 43, 45, 47 or 49.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98. The substitution may be conservative, which means the replacement of a certain amino acid residue by another residue having similar physical and chemical characteristics. Non-limiting examples of conservative substitution include replacement between aliphatic group-containing amino acid residues such as lie, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p.6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of SEQ ID NO:1, 3, 5, 7, 9, 11, 13, 15, 17, 19, 21, 23, 25, 27, 29, 31, 33, 35, 37, 39, 41, 43, 45, 47 or 49. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of SEQ ID NO:1, 3, 5, 7, 9, 11, 13, 15, 17, 19, 21, 23, 25, 27, 29, 31, 33, 35, 37, 39, 41, 43, 45, 47 or 49.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and Suppression DNA Constructs:

In one aspect, the present disclosure includes suppression DNA constructs.

One embodiment is a suppression DNA construct comprising a polynucleotide, wherein the polynucleotide is operably linked to a heterologous promoter in sense or antisense orientation, or both, wherein the construct is effective for reducing expression of an endogenous YEP6 gene in a plant, and wherein the polynucleotide comprises: (a) the nucleotide sequence of SEQ ID NO:1, 3, 5, 7, 9, 11, 13, 15, 17, 19, 21, 23, 25, 27, 29, 31, 33, 35, 37, 39, 41, 43, 45, 47 or 49; (b) a nucleotide sequence that has at least 80% sequence identity, when compared to SEQ ID NO:1, 3, 5, 7, 9, 11, 13, 15, 17, 19, 21, 23, 25, 27, 29, 31, 33, 35, 37, 39, 41, 43, 45, 47 or 49; (c) a nucleotide sequence of at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49; (d) a nucleotide sequence that can hybridize under stringent conditions with the nucleotide sequence of (a); or (e) a modified plant miRNA precursor, wherein the precursor has been modified to replace the miRNA encoding region with a sequence designed to produce a miRNA directed to SEQ ID NO:1, 3, 5, 7, 9, 11, 13, 15, 17, 19, 21, 23, 25, 27, 29, 31, 33, 35, 37, 39, 41, 43, 45, 47 or 49.

One embodiment of the current disclosure encompasses the suppression DNA construct, wherein the polynucleotide comprises at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49, and the suppression DNA construct is designed for RNA interference, and is effective for reducing expression of YEP6 gene in a plant. In one embodiment, the polynucleotide comprises a nucleotide sequence that has at least 90% sequence identity to SEQ ID NO:55.

In another embodiment, the YEP6 polypeptide may be from a monocot plant.

In one embodiment, the YEP6 polypeptide may be from Zea mays, Glycine max, Oryza sativa, Sorghum bicolor, Saccharum officinarum, or Triticum aestivum.

In one embodiment, the promoter may be a constitutive promoter, an inducible promoter, a tissue-specific promoter.

A suppression DNA construct may comprise at least one regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, and combinations thereof, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a YEP6 polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9, 11, 13, 15, 17, 19, 21, 23, 25, 27, 29, 31, 33, 35, 37, 39, 41, 43, 45, 47 or 49, and combinations thereof, or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” generally refers to the production of antisense RNA transcripts capable of suppressing the expression of the target gene or gene product. “Antisense RNA” generally refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Sense suppression” generally refers to the production of sense RNA transcripts capable of suppressing the expression of the target gene or gene product. “Sense” RNA generally refers to RNA transcript that includes the mRNA and can be translated into protein within a cell or in vitro. Sense constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference generally refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

In some embodiments, the RNA interference is achieved by hairpin RNA interference or intron containing hairpin RNA (hpRNA) (Waterhouse and Helliwell, (2003) Nat. Rev. Genet. 5:29-38). For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous YEP6 mRNA whose expression is to be inhibited, and an antisense sequence that is fully or partially complementary to the sense sequence. Such kind of hairpin RNA interference is highly efficient at inhibiting the expression of endogenous genes (for example US Patent publication No. 20030175965; Meyerowitz (2000) Proc. Natl. Acad. Sci. USA 97:5985-5990). In some embodiments, the hpRNA molecule comprises an intron that is capable of being spliced in the cell in which the hpRNA is expressed. The use of an intron minimizes the size of the loop in the hairpin RNA molecule following splicing, and this increases the efficiency of interference. Methods of using intron hpRNAi to inhibit expression of endogenous plant genes have been described in literature, such as US patent publication number 20030180955; Waterhouse and Helliwell, (2003) Nat. Rev. Genet. 5:29-38, all of which are incorporated herein by reference). A number of introns have been tested for intron containing hpRNA interference constructs such as petunia chalcone synthase intron, rice waxy intron, Flavaria trinervia pyruvate orthophosphate dikinase intron, intron from potato LS1 gene (Smith et al. (2000) Nature 407:319-320; Preuss and Pikaard Targeted gene silencing in plants using RNA interference Pg. 23-36; from “RNA Interference˜Nuts and bolts of siRNA technology”; edited by David Engelke, Eckes et al (1986) Mol. Gen Genet. 205:14-22). In one embodiment, the intron could be the 2^{nd} intron from potato LS1 gene.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA* sequence” are used interchangeably herein and they refer to a sequence in the miRNA precursor that is highly complementary to the miRNA sequence. The miRNA and miRNA* sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot.

In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

Promoters for use also include the following: Zm-GOS2 (maize promoter for “Gene from Oryza sativa”, US publication number US2012/0110700 Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters.

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1 BIO promoter (WO06055487, published May 26, 2006), the CRWAQ81 (WO05035770, published Apr. 21, 2005) and the maize ZRP2.47 promoter (NCBI accession number: U38790; GI No. 1063664),

Suppression DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. Appln. No. 61/514,055), Actin terminator, Os-Actin terminator, Ubi terminator, Sb-Ubi terminator, Os-Ubi terminator.

Any plant can be selected for the identification of regulatory sequences and YEP6 polypeptide genes to be used in suppression DNA constructs and other compositions (e.g. transgenic plants, seeds and cells) and methods of the present disclosure. Examples of suitable plants for the isolation of genes and regulatory sequences and for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yarns, and zucchini.

Compositions:

A composition of the present disclosure includes a transgenic microorganism, cell, plant, and seed comprising the suppression DNA construct. The cell may be eukaryotic, e.g., a yeast, insect or plant cell, or prokaryotic, e.g., a bacterial cell.

A composition of the present disclosure is a plant comprising in its genome any of the suppression DNA constructs of the present disclosure (such as any of the constructs discussed above). Compositions also include any progeny of the plant, and any seed obtained from the plant or its progeny, wherein the progeny or seed comprises within its genome the suppression DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced suppression DNA construct. These seeds can be grown to produce plants that would exhibit an altered agronomic characteristic (e.g., an increased agronomic characteristic optionally under stress conditions), or used in a breeding program to produce hybrid seed, which can be grown to produce plants that would exhibit such an altered agronomic characteristic. The seeds may be maize seeds. The stress condition may be selected from the group of drought stress, and nitrogen stress.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

Particular embodiments include but are not limited to the following: A plant (for example, a maize, rice or sorghum plant) comprising in its genome any of the suppression DNA constructs described herein.

A plant comprising a disruption or silencing of at least one of the YEP6 genes.

A plant (for example, a maize, rice or sorghum plant) comprising in its genome any of the suppression DNA constructs described herein, wherein said plant exhibits at least one phenotype selected from the group consisting of increased staygreen phenotype, increased yield, increased biomass and increased tolerance to abiotic stress, when compared to a control plant not comprising said recombinant DNA construct. The abiotic stress may be drought stress, low nitrogen stress, or both. The plant may further exhibit an alteration of at least one agronomic characteristic when compared to the control plant.

A plant with lower expression or activity levels of at least one endogenous YEP6 gene or polypeptide, when compared to a control plant, wherein the reduction in expression of the endogenous YEP6 gene is caused by sense suppression, antisense suppression, miRNA suppression, ribozymes, or RNA interference. In one embodiment, the plant of the current disclosure can have the reduction in expression of the endogenous YEP6 gene caused by a mutation in the endogenous YEP6 gene. In one embodiment, the mutation in the endogenous YEP6 gene in the plant is caused by insertional mutagenesis. In one embodiment, the insertional mutagenesis is caused by transposon mutagenesis.

A plant (for example, a maize, rice or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, or (b) a full complement of the nucleic acid sequence of (a), and wherein said plant exhibits an alteration of at least one agronomic characteristic when compared to a control plant not comprising said suppression DNA construct.

Any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the YEP6 polypeptide may be from Zea mays, Glycine max, Glycine tabacina, Glycine soja, Glycine tomentella, Oryza sativa, Brassica napus, Sorghum bicolor, Saccharum officinarum, or Triticum aestivum.

In any of the embodiments described herein, the suppression DNA construct may comprise at least a promoter functional in a plant as a regulatory sequence.

In any of the embodiments described herein or any other embodiments of the present disclosure, the alteration of at least one agronomic characteristic is either an increase or decrease.

In any of the embodiments described herein, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. For example, the alteration of at least one agronomic characteristic may be an increase in yield, greenness or biomass.

In any of the embodiments described herein, the plant encompassed by the current disclosure, and comprising disruption or silencing of at least one endogenous YEP6 gene may exhibit the alteration of at least one agronomic characteristic when compared, under at least one stress condition, to a control plant. The at least one stress condition may be either drought stress, low nitrogen stress, or both.

In one embodiment, the plant is a hybrid plant exhibiting staygreen phenotype

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

In any of the embodiments described herein, the plant may exhibit less yield loss relative to the control plants, for example, at least 25%, at least 20%, at least 15%, at least 10% or at least 5% less yield loss, under stress conditions. The stress may be either drought stress, low nitrogen stress, or both.

In one embodiment, the plant may exhibit increased yield, for example, at least 5%, at least 10%, at least 15%, at least 20% or at least 25% increased yield, relative to the control plants under non-stress conditions.

Yield analysis can be done to determine whether plants that have downregulated expression levels of at least one of the YEP6 genes have an improvement in yield performance under non-stress or stress conditions, when compared to the control plants that have wild-type expression levels and activity levels of the YEP gene and polypeptide, respectively. Stress conditions can be water-limiting conditions, or low nitrogen conditions. Specifically, drought conditions or nitrogen limiting conditions can be imposed during the flowering and/or grain fill period for plants that contain the suppression DNA construct and the control plants.

In one embodiment, the plant may exhibit increased staygreen phenotype, or an increase in biomass, relative to the control plants under non-stress conditions.

In one embodiment, the plant may exhibit increased staygreen phenotype, or an increase in biomass, relative to the control plants under stress conditions.

In one embodiment, yield can be measured in many ways, including, for example, test weight, seed weight, seed number per plant, seed number per unit area (i.e. seeds, or weight of seeds, per acre), bushels per acre, tonnes per acre, tons per acre, kilo per hectare.

The terms “stress tolerance” or “stress resistance” as used herein generally refers to a measure of a plants ability to grow under stress conditions that would detrimentally affect the growth, vigor, yield, and size, of a “non-tolerant” plant of the same species. Stress tolerant plants grow better under conditions of stress than non-stress tolerant plants of the same species. For example, a plant with increased growth rate, compared to a plant of the same species and/or variety, when subjected to stress conditions that detrimentally affect the growth of another plant of the same species would be said to be stress tolerant. A plant with “increased stress tolerance” can exhibit increased tolerance to one or more different stress conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

“Drought” generally refers to a decrease in water availability to a plant that, especially when prolonged, can cause damage to the plant or prevent its successful growth (e.g., limiting plant growth or seed yield). “Water limiting conditions” generally refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

Typically, when a transgenic plant comprising a suppression DNA construct in its genome exhibits increased stress tolerance relative to a reference or control plant, the reference or control plant does not comprise in its genome the suppression DNA construct.

The range of stress and stress response depends on the different plants which are used, i.e., it varies for example between a plant such as wheat and a plant such as Arabidopsis.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well-watered treatments of transgenic plants and relevant control plants:

The variable “% area chg_start chronic−acute2” is a measure of the percent change in total area determined by remote visible spectrum imaging between the first day of chronic stress and the day of the second acute stress.

The variable “% area chg_start chronic−end chronic” is a measure of the percent change in total area determined by remote visible spectrum imaging between the first day of chronic stress and the last day of chronic stress.

The variable “% area chg_start chronic−harvest” is a measure of the percent change in total area determined by remote visible spectrum imaging between the first day of chronic stress and the day of harvest.

The variable “% area chg_start chronic−recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress−(variable fluorescence difference between the maximum and minimum fluorescence/maximum fluorescence)

The variable “fv/fm_acute2” is a measure of the optimum quantum yield (Fv/Fm) at the end of the second acute stress−(variable flourescence difference between the maximum and minimum fluorescence/maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by Lemna Tec Instrument) over a single day (Y(t)=Y0*e^{r*t}). Y(t)=Y0*e^{r*t} is equivalent to % change in Y/Δ t where the individual terms are as follows: Y(t)=Total surface area at t; Y0=Initial total surface area (estimated); r=Specific Growth Rate day⁻¹, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a suppression DNA construct, such that the progeny are segregating into plants either comprising or not comprising the suppression DNA construct: the progeny comprising the suppression DNA construct would be typically measured relative to the progeny not comprising the suppression DNA construct (i.e., the progeny not comprising the suppression DNA construct is the control or reference plant). The progeny comprising the suppression DNA construct would have a disruption or silencing of at least one YEP6 gene.

2. Introgression of a suppression DNA construct into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a or suppression DNA construct: the second hybrid line would typically be measured relative to the first hybrid line (i.e., the first hybrid line is the control or reference plant).

4. A plant comprising a suppression DNA construct: the plant may be assessed or measured relative to a control plant not comprising the suppression DNA construct but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the suppression DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing yield in a plant, method of increasing staygreen phenotype in a plant, method of increasing drought tolerance in a plant, methods for altering an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of introducing into a plant a suppression DNA construct comprising a polynucleotide operably linked to a heterologous promoter, wherein the suppression DNA construct is effective for reducing expression of an endogenous YEP6 gene. In one embodiment, the suppression DNA construct is selected from the group consisting of: sense suppression construct, antisense suppression construct, ribozyme construct, RNA interference construct and an miRNA construct. In one embodiment, the suppression DNA construct is an RNA interference construct and the RNA interference construct comprises at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49, and wherein the RNA interference construct is effective for reducing the expression of the endogenous YEP6 gene. In one embodiment, the RNA interference construct comprises a polynucleotide sequence that has at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:55.

A method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of: (a) introducing a mutation into an endogenous YEP6 gene; and (b) detecting said mutation using the Targeted Induced Local Lesions In Genomics (TILLING) method, wherein said mutation results in reducing expression of the endogenous YEP6 gene.

A method of enhancing seed yield in a plant, when compared to a control plant, wherein the plant exhibits enhanced yield under either stress conditions, or non-stress conditions, or both, the method comprising the step of reducing expression of the endogenous YEP6 gene in a plant.

A method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the step of utilizing a transposon to introduce an insertion into an endogenous YEP6 gene in a plant, wherein the insertion is effective for reducing expression of an endogenous YEP6 gene.

A method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing into a plant a suppression DNA construct comprising a polynucleotide operably linked to a heterologous promoter, wherein the polynucleotide encodes a fragment or a variant of a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the fragment or the variant confers a dominant-negative phenotype in the plant.

A method of making a plant in which activity of an endogenous YEP6 polypeptide is reduced, when compared to the activity of wild-type YEP6 polypeptide from a control plant, and wherein the plant exhibits at least one phenotype selected from the group consisting of: increased yield, increased staygreen, increased abiotic stress tolerance and increased biomass, compared to the control plant, wherein the method comprises the steps of introducing a mutation in an endogenous YEP6 gene, wherein the mutation is effective for reducing the activity of the endogenous YEP6 polypeptide. In one embodiment, the method further comprises the step of detecting the mutation and the detection is done using the Targeted Induced Local Lesions IN Genomics (TILLING) method.

The current disclosure also includes the plant obtained by any of the methods disclosed herein, wherein the plant exhibits at least one phenotype selected from the group consisting of: increased yield, increased staygreen, increased abiotic stress tolerance and increased biomass, compared to the control plant.

The current disclosure also includes a method for transforming a cell (or microorganism) comprising transforming a cell (or microorganism) with any of the isolated polynucleotides or suppression DNA constructs of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or suppression DNA constructs of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a suppression DNA construct comprising a polynucleotide of the disclosure operably linked to at least one regulatory sequence, and wherein the transformed host cell is grown under conditions that are suitable for expression of the suppression DNA construct.

A method of altering the level of expression of a polypeptide of the disclosure in a host cell comprising: (a) transforming a host cell with a suppression DNA construct of the present disclosure; and (b) growing the transformed host cell under conditions that are suitable for expression of the suppression DNA construct wherein expression of the suppression DNA construct results in production of altered levels of expression or activity of the polypeptide of the disclosure in the transformed host cell.

The method may further comprise (c) obtaining a progeny plant derived from the transgenic plant, wherein said progeny plant comprises in its genome the suppression DNA construct and exhibits at least one phenotype selected from the group consisting of: increased yield, increased staygreen and increased stress tolerance, wherein the stress is selected from the group consisting of drought stress, and low nitrogen stress, when compared to a control plant not comprising the suppression DNA construct. The progeny plant further exhibits a lower level of expression and/or activity of at least one YEP6 gene and/or polypeptide.

A method of increasing stress tolerance, wherein the stress is selected from the group consisting of drought stress, and low nitrogen stress, the method comprising: (a) introducing into a regenerable plant cell a suppression DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1, 3, 5, 7, 9, 11, 13, 15, 17, 19, 21, 23, 25, 27, 29, 31, 33, 35, 37, 39, 41, 43, 45, 47 or 49; or (b) derived from SEQ ID NO:1, 3, 5, 7, 9, 11, 13, 15, 17, 19, 21, 23, 25, 27, 29, 31, 33, 35, 37, 39, 41, 43, 45, 47 or 49, by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits increased stress tolerance, wherein the stress is selected from the group consisting of drought stress, and low nitrogen stress, when compared to a control plant not comprising the suppression DNA construct. The method may further comprise (c) obtaining a progeny plant derived from the transgenic plant, wherein said progeny plant comprises in its genome the suppression DNA construct and exhibits increased stress tolerance, wherein the stress is selected from the group consisting of drought stress and low nitrogen stress, when compared to a control plant not comprising the recombinant DNA construct.

A method of selecting for (or identifying) increased stress tolerance in a plant, wherein the stress is selected from the group consisting of drought stress and low nitrogen stress, the method comprising (a) obtaining a transgenic plant, wherein the transgenic plant comprises in its genome a suppression DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98; (b) obtaining a progeny plant derived from said transgenic plant, wherein the progeny plant comprises in its genome the recombinant DNA construct; and (c) selecting (or identifying) the progeny plant with increased stress tolerance, wherein the stress is selected from the group consisting of drought stress and low nitrogen stress, compared to a control plant not comprising the suppression DNA construct.

In another embodiment, a method of selecting for (or identifying) increased stress tolerance in a plant, wherein the stress is selected from the group consisting of drought stress, and low nitrogen stress, the method comprising: (a) obtaining a transgenic plant, wherein the transgenic plant comprises in its genome a suppression DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98; (b) growing the transgenic plant of part (a) under conditions wherein the polynucleotide is expressed; and (c) selecting (or identifying) the transgenic plant of part (b) with increased stress tolerance, wherein the stress is selected from the group consisting of drought stress, and low nitrogen stress, compared to a control plant not comprising the suppression DNA construct. The transgenic plant comprising the suppression DNA construct further has reduced levels of expression of at least one YEP6 gene, and/or reduced levels of activity of at least one YEP6 polypeptide.

A method of selecting for (or identifying) increased stress tolerance in a plant, wherein the stress is selected from the group consisting of drought stress, triple stress and osmotic stress the method comprising: (a) obtaining a transgenic plant, wherein the transgenic plant comprises in its genome a suppression DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1, 3, 5, 7, 9, 11, 13, 15, 17, 19, 21, 23, 25, 27, 29, 31, 33, 35, 37, 39, 41, 43, 45, 47 or 49; or (ii) derived from SEQ ID NO:1, 3, 5, 7, 9, 11, 13, 15, 17, 19, 21, 23, 25, 27, 29, 31, 33, 35, 37, 39, 41, 43, 45, 47 or 49 by alteration of one or more nucleotides by at least one method selected from the group consisting of: deletion, substitution, addition and insertion; (b) obtaining a progeny plant derived from said transgenic plant, wherein the progeny plant comprises in its genome the suppression DNA construct; and (c) selecting (or identifying) the progeny plant with increased drought tolerance, when compared to a control plant not comprising the suppression DNA construct.

A method of selecting for (or identifying) an alteration of an agronomic characteristic in a plant, comprising (a) obtaining a transgenic plant, wherein the transgenic plant comprises in its genome a suppression DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98; (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under at least one stress condition, to a control plant not comprising the suppression DNA construct. The at least one stress condition may be selected from the group of drought stress, and low nitrogen stress. The polynucleotide preferably encodes a YEP6 polypeptide.

In another embodiment, a method of selecting for (or identifying) an alteration of at least one agronomic characteristic in a plant, comprising: (a) obtaining a transgenic plant, wherein the transgenic plant comprises in its genome a suppression DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98, wherein the transgenic plant comprises in its genome the suppression DNA construct; (b) growing the transgenic plant of part (a) under conditions wherein the polynucleotide is expressed; and (c) selecting (or identifying) the transgenic plant of part (b) that exhibits an alteration of at least one agronomic characteristic when compared to a control plant not comprising the suppression DNA construct. Optionally, said selecting (or identifying) step (c) comprises determining whether the transgenic plant exhibits an alteration of at least one agronomic characteristic when compared, under at least one condition, to a control plant not comprising the suppression DNA construct. The at least one agronomic trait may be yield, biomass, or both and the alteration may be an increase. The at least one stress condition may be selected from the group of drought stress, and low nitrogen stress.

A method of selecting for (or identifying) an alteration of an agronomic characteristic in a plant, comprising (a) obtaining a transgenic plant, wherein the transgenic plant comprises in its genome a suppression DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1, 3, 5, 7, 9, 11, 13, 15, 17, 19, 21, 23, 25, 27, 29, 31, 33, 35, 37, 39, 41, 43, 45, 47 or 49 by alteration of one or more nucleotides by at least one method selected from the group consisting of: deletion, substitution, addition and insertion; (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under stress conditions, wherein the stress is selected from the group consisting of drought stress, and low nitrogen stress, to a control plant not comprising the suppression DNA construct. The polynucleotide preferably encodes a YEP6 polypeptide.

A method of producing seed (for example, seed that can be sold as a drought tolerant product offering) comprising any of the preceding methods, and further comprising obtaining seeds from said progeny plant, wherein said seeds comprise in their genome said suppression DNA construct.

Another embodiment is a method of identifying one or more trait loci or a gene controlling such trait loci, the method comprising: (a) developing a breeding population of maize plants, wherein the breeding population is generated by crossing a first maize inbred line characterized as a high protein line with a second maize inbred line characterized as a low protein line; (b) selecting a plurality of progeny maize plants based on at least one phenotype of interest selected from the group consisting of delayed senescence, increased nitrogen use efficiency, increased yield, increased abiotic stress tolerance, increased staygreen, and increased biomass; (c) performing marker analysis for the one or more phenotypes identified in the progeny of plants; and (d) identifying the trait loci or the gene controlling the trait loci.

In any of the preceding methods or any other embodiments of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. The alteration of at least one agronomic characteristic may be an increase in yield, greenness or biomass.

In any of the preceding methods or any other embodiments of methods of the present disclosure, the plant may exhibit the alteration of at least one agronomic characteristic when compared, under stress conditions, wherein the stress is selected from the group consisting of drought stress, and low nitrogen stress, to a control plant not comprising said suppression DNA construct.

In any of the preceding methods or any other embodiments of methods of the present disclosure, alternatives exist for introducing into a regenerable plant cell a suppression DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of suppression DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Identification and Cloning of a Leaf-Senescence and N-Remobilization QTL

High Protein (HP) and Low Protein (LP) inbred lines were derived from a long-term selection experiment. The lines were crossed, and the progeny were selfed for a number of generations to generate populations for multiple purposes.

In an HP×LP population of 90 F₆ families, a clear segregation in senescence of the first leaf was observed in 4-week-old seedlings (V4 stage). The leaf senescence phenotype was scored visually (1=HP-like, fully senescenced/yellow, 3=LP-like, not senescenced/green). As such, the HP×LP population was used to identify QTL associated with leaf senescence using a traditional linkage mapping approach. A major QTL was detected on chromosome 3 between 66.1 cM and 125.4 cM on a single meiosis based genetic map (181.7-411.6 cM on an IBM2 map) using a single-marker analysis of 239 polymorphic SNP markers and WinQTL Cartographer. To confirm and further refine the QTL interval, 270 F₆ families from the same population were phenotyped and 39 plants exhibiting extreme phenotypes were selected for higher resolution mapping. The QTL was further delimited to the interval between 80.8 cM and 84.4 cM on a single meiosis based genetic map. The leaf senescence phenotype was re-named as N-remobilization, as it was speculated that the earlier senescing phenotype of the old leaf in HP is caused by more rapid nitrogen remobilization from older leaves to younger leaves.

In an effort to fine-map and clone the N-remobilization QTL, three F₆ plants which are heterozygous across the QTL interval (“residual heterozygosity”) and their progenies were selected for self-pollination to generate a large mapping population. 590 individuals were initially genotyped with markers located between 77.2 cM and 87.6 cM on a single meiosis based genetic map (230.1 and 313.4 on an IBM2 map) and 141 recombinants were identified. Subsequently, 3397 individual plants were genotyped with markers between 79.5 cM and 83.1 cM on a single meiosis based genetic map, and 628 recombinants were identified. The recombinant plants were self-pollinated and their progenies were scored for the leaf senescence phenotype, as described above. Additional SNP markers were developed within the QTL interval to genotype the recombinants. The N-remobilization QTL was eventually narrowed down to a 37.4 kb interval, flanked by 3NR_29 (2 recombinants) (amplicon obtained using primers having SEQ ID NOS:51 and 52) and 3NR_72 (9 recombinants) (amplicon obtained using primers having SEQ ID NOS:53 and 54). There is a single annotated protein-coding gene (with a nucleotide coding sequence set forth in SEQ ID NO:1) encoding a NAC-domain containing protein (SEQ ID NO:2) within this interval. The genotypes of this gene in all the recombinants segregate perfectly with the phenotypes. Therefore, it is the candidate gene for the N-remobilization QTL. This NAC-domain containing maize gene was named ZmYEP6.

### Example 2

### Construction of a Suppression DNA Construct

A transgenic loss of function approach was used to elucidate the function of ZmYEP6 (the maize NAC gene identified in Example 1; SEQ ID NO:1). A suppression DNA construct containing a a 310 bp fragment (nucleotides 212 to 522 of the coding sequence; SEQ ID NO:55) of the coding sequence of ZmYEP6 (SEQ ID NO:1), used in sense and antisense orientation with potato LS intron2 (ST-LS Intron2; In US20120058245) as a spacer, was constructed. The RNAi cassette with inverted repeats was driven by the Zm-UBI promoter and was operably linked to the Sb-GKAF terminator. The plasmid vector PHP52729 containing the suppression DNA construct (FIG. 1) also contained UBI:PMI and OsACT:MOPAT (MOPAT driven by Oryza sativa Actin promoter) as selectable markers along with LTP2:DSRED for transgenic seed sorting.

### Example 3

### Introduction of Suppression DNA Construct into Agrobacterium tumefaciens LBA4404 by Electroporation

Plasmid vector PHP52729 was introduced into Agrobacterium by electroporation.

In this standard method, electroporation competent cells (40 μL), such as Agrobacterium tumefaciens LBA4404 containing PHP10523 (PCT Publication No. WO/2012/058528), are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2×YT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates some contaminating colonies observed when using poorer preparations of LBA4404 competent cells.

Option 2: Perform two replicates of the electroporation to compensate for poorer electrocompetent cells.

Identification of Transformants:

Four independent colonies are picked and streaked on plates containing AB minimal medium and 50 μg/mL spectinomycin for isolation of single colonies. The plates are incubated at 28° C. for two to three days. A single colony for each putative co-integrate is picked and inoculated with 4 mL of 10 g/L bactopeptone, 10 g/L yeast extract, 5 g/L sodium chloride and 50 mg/L spectinomycin. The mixture is incubated for 24 h at 28° C. with shaking. Plasmid DNA from 4 mL of culture is isolated using QIAGEN® Miniprep and an optional Buffer PB wash. The DNA is eluted in 30 μL. Aliquots of 2 μL are used to electroporate 20 μL of DH10b+20 μL of twice distilled H₂O as per above. Optionally a 15 μL aliquot can be used to transform 75-100 μL of INVITROGEN™ Library Efficiency DH5α. The cells are spread on plates containing LB medium and 50 μg/mL spectinomycin and incubated at 37° C. overnight.

Three to four independent colonies are picked for each putative co-integrate and inoculated 4 mL of 2×YT medium (10 g/L bactopeptone, 10 g/L yeast extract, 5 g/L sodium chloride) with 50 μg/mL spectinomycin. The cells are incubated at 37° C. overnight with shaking. Next, isolate the plasmid DNA from 4 mL of culture using QIAprep® Miniprep with optional Buffer PB wash (elute in 50 μL). Use 8 μL for digestion with Sail (using parental DNA and PHP10523 as controls). Three more digestions using restriction enzymes BamHI, EcoRI, and HindIII are performed for 4 plasmids that represent 2 putative co-integrates with correct Sail digestion pattern (using parental DNA and PHP10523 as controls). Electronic gels are recommended for comparison.

### Example 4

### Transformation of Maize Using Agrobacterium

Agrobacterium tumefaciens containing the suppression DNA construct described in Example 2 was used to transform corn with plasmid PHP52729 via Agrobacterium-mediated transformation in order to examine the resulting phenotype.

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium.

Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 pE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringone, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a suppression DNA construct can be introduced into an elite maize inbred line either by direct transformation or introgression from a separately transformed line.

Transgenic plants, either inbred or hybrid, can undergo more vigorous field-based experiments to study yield enhancement and/or stability under water limiting and water non-limiting conditions.

Subsequent yield analysis can be done to determine whether plants that contain the reduced expression levels or reduced activity of YEP6 genes have an improvement in yield performance (under stress or non-stress conditions), when compared to the control (or reference) plants that do not contain the suppression DNA construct. Specifically, water limiting conditions can be imposed during the flowering and/or grain fill period for plants that have reduced expression or activity levels of the YEP6 gene, and the control plants.

### Example 5A

### Identification of cDNA Clones

cDNA clones encoding YEP6 polypeptides can be identified by conducting BLAST (Basic Local Alignment Search Tool; Altschul et al. (1993) J. Mol. Biol. 215:403-410; see also the explanation of the BLAST algorithm on the world wide web site for the National Center for Biotechnology Information at the National Library of Medicine of the National Institutes of Health) searches for similarity to amino acid sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The DNA sequences from clones can be translated in all reading frames and compared for similarity to all publicly available protein sequences contained in the “nr” database using the BLASTX algorithm (Gish and States (1993) Nat. Genet. 3:266-272) provided by the NCBI. The polypeptides encoded by the cDNA sequences can be analyzed for similarity to all publicly available amino acid sequences contained in the “nr” database using the BLASTP algorithm provided by the National Center for Biotechnology Information (NCBI). For convenience, the P-value (probability) or the E-value (expectation) of observing a match of a cDNA-encoded sequence to a sequence contained in the searched databases merely by chance as calculated by BLAST are reported herein as “pLog” values, which represent the negative of the logarithm of the reported P-value or E-value. Accordingly, the greater the pLog value, the greater the likelihood that the cDNA-encoded sequence and the BLAST “hit” represent homologous proteins.

ESTs sequences can be compared to the Genbank database as described above. ESTs that contain sequences more 5- or 3-prime can be found by using the BLASTN algorithm (Altschul et al (1997) Nucleic Acids Res. 25:3389-3402) against the DUPONT™ proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described above. Homologous genes belonging to different species can be found by comparing the amino acid sequence of a known gene (from either a proprietary source or a public database) against an EST database using the TBLASTN algorithm. The TBLASTN algorithm searches an amino acid query against a nucleotide database that is translated in all 6 reading frames. This search allows for differences in nucleotide codon usage between different species, and for codon degeneracy.

In cases where the sequence assemblies are in fragments, the percent identity to other homologous genes can be used to infer which fragments represent a single gene. The fragments that appear to belong together can be computationally assembled such that a translation of the resulting nucleotide sequence will return the amino acid sequence of the homologous protein in a single open-reading frame. These computer-generated assemblies can then be aligned with other polypeptides disclosed herein.

The coding sequences of the cDNA clones encoding maize YEP6 polypeptides are provided as SEQ ID NOs: 3, 5, 7, 9, 11, 13, 15, 17, 19, 21, 23, 25, 27, 29, 31, 33, 35, 37, 39, 41, 43, 45, 47, and 49. The respective encoded polypeptides are provided as SEQ ID Nos: 4, 6, 8, 10, 12, 14, 16, 18, 20, 22, 24, 26, 28, 30, 32, 34, 36, 38, 40, 42, 44, 46, 48, and 50 (as shown in Table 1).

### Example 5B

### Identification of Orthologous YEP6 Polypeptides

Sequences homologous to the ZmYEP6 polypeptide (SEQ ID NO:2) that contains the NAM domain (PF02365) were identified in rice and in sorghum using the profile hidden Markov models (HMMs) search program pfam_scan against Pfam database 26.0. Phylogenetic analysis was performed for all NAC genes from rice and sorghum, separately. A subset of 18 rice genes and 24 sorghum genes belonging to the same clade as ZmYEP6 was selected (SEQ ID NOs:57-98).

### Example 5C

### Sequence Alignment and Percent Identity Calculations for YEP6 Polypeptides

Sequence alignments and percent identity calculations may be performed using the MEGALIGN® program of the LASERGENE® bioinformatics computing suite (DNASTAR Inc., Madison, Wis.). Multiple alignment of the sequences may be performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE=1, GAP PENALTY=3, WINDOW=5 and DIAGONALS SAVED=5.

FIGS. 2A-2J show the alignment of the YEP6 polypeptides from Zea mays that are clustered in clade 1 of the phylogenetic tree for NAC polypeptides (FIG. 4). This includes ZmYEP6 (SEQ ID NO:2) and its maize homologs SEQ ID NOs:4, 6, 8, 10, 12, 14, 16, 18, 20, 22, 24, 26, 28, 30, 32, 34, 36, 38, 40, 42, 44, 46, 48 and 50. FIGS. 3A through 3D show the percent sequence identity and divergence values for each pair of amino acid sequences of the Zea mays YEP6 polypeptides displayed in FIGS. 2A-FIG. 2J. Percent similarity scores are shown in bold, while the percent divergence scores are shown in italics.

### Example 6

### Yield Analysis of Maize Lines Containing a Suppression Construct Comprising a Zea mays YEP6 Gene

A suppression DNA construct comprising a fragment or entire sequence of SEQ ID NO:1 or any of the Zea mays YEP6 genes (SEQ ID NOs: 3, 5, 7, 9, 11, 13, 15, 17, 19, 21, 23, 25, 27, 29, 31, 33, 35, 37, 39, 41, 43, 45, 47, and 49) can be introduced into an elite maize inbred line either by direct transformation or introgression from a separately transformed line.

Transgenic plants, either inbred or hybrid, can undergo more vigorous field-based experiments to study yield enhancement and/or stability under stress and non-stress conditions.

Subsequent yield analysis can be done to determine whether plants that have downregulated expression levels of YEP6 gene have an improvement in yield performance under non-stress or stress conditions, when compared to the control plants that have wild-type expression levels and activity levels of the YEP6 gene and polypeptide, respectively. Stress conditions can be water-limiting conditions, or low nitrogen conditions. Specifically, drought conditions or nitrogen limiting conditions can be imposed during the flowering and/or grain fill period for plants that contain the suppression DNA construct and the control plants. Reduction in yield can be measured for both. Plants with reduced expression levels of the YEP6 gene have less yield loss relative to the control plants, for example, at least 25%, at least 20%, at least 15%, at least 10% or at least 5% less yield loss.

The above method may be used to select transgenic plants with increased yield, under non-stress conditions, when compared to a control plant. Plants containing the reduced expression or activity levels of YEP6 gene or polypeptide, may have increased yield, under non-stress conditions, relative to the control plants, for example, at least 5%, at least 10%, at least 15%, at least 20% or at least 25% increased yield.

### Example 7

### Yield Analysis of Transgenic Events Containing PHP52729 in Field Plots 1^{st} Year Testing

Transgenic events of PHP52729 (See Example 2) were molecularly characterized for transgene copy number and expression by genomic PCR and RT-PCR, respectively. Events containing single copy of transgene with detectable transgene expression were advanced for field testing. Test crosses (hybrid seeds) were produced and tested in the field in multi-locations/replications experiments both in normal (6 locations; FIG. 4A) and low N (3 locations, FIG. 4B) fields. Transgenic events were evaluated in field plots under normal nitrogen conditions and under low nitrogen conditions, where fertilizer application is reduced by 30% or more.

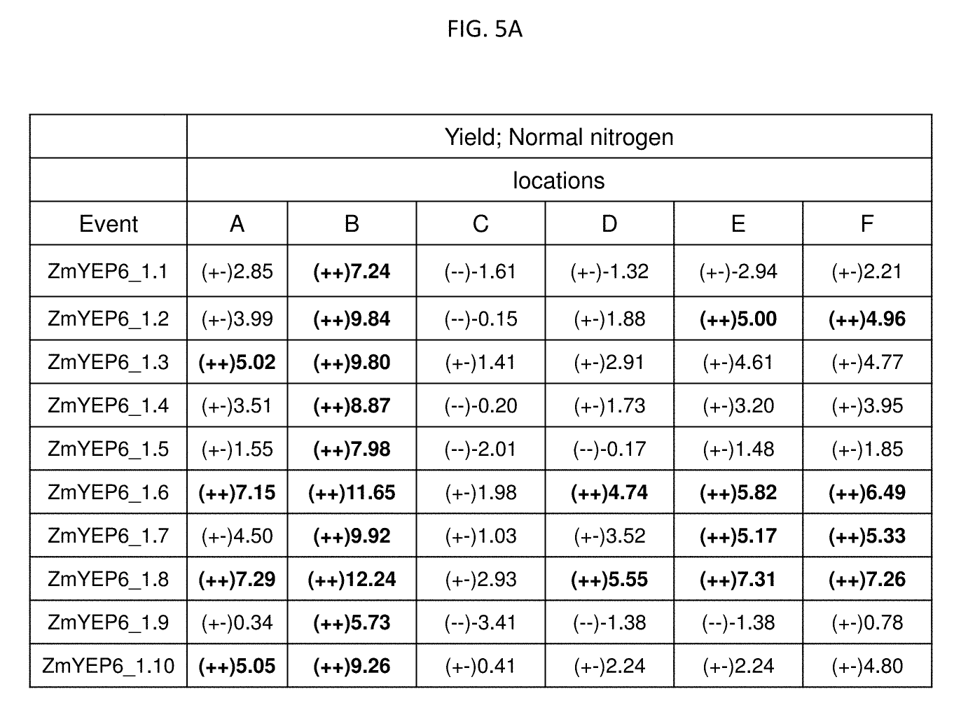

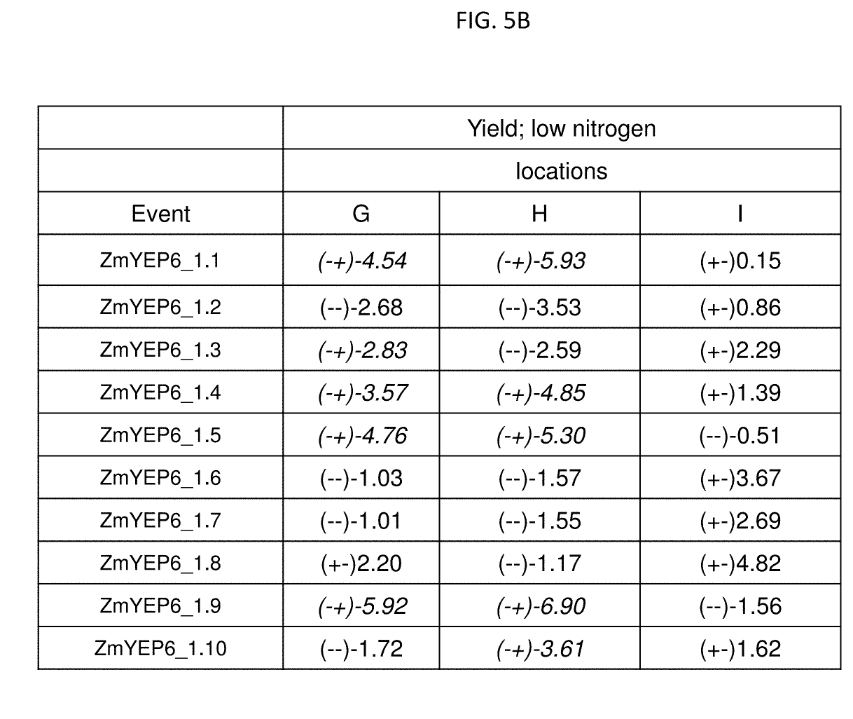

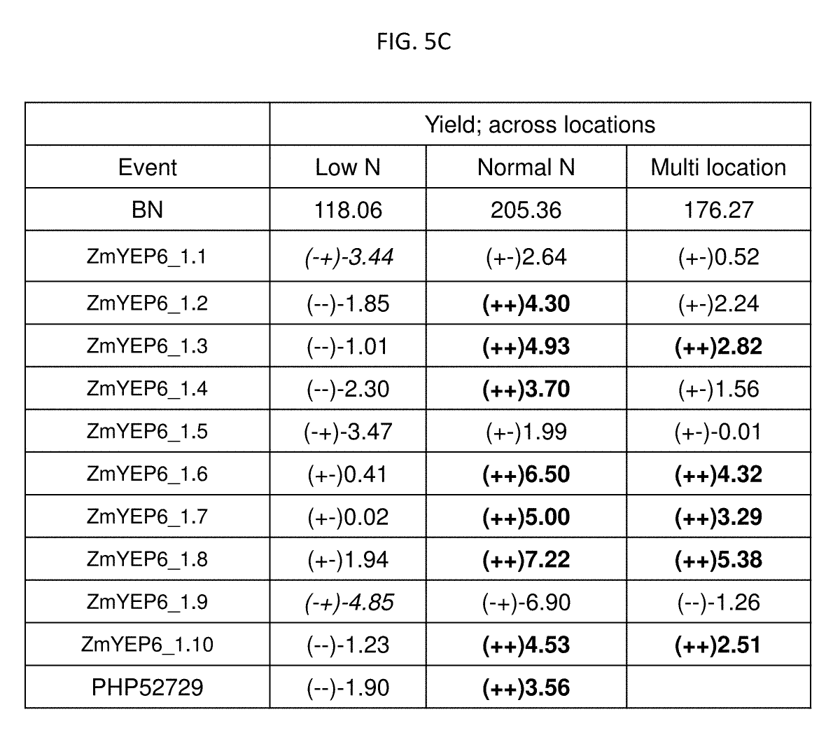

Yield data was collected in all locations, with 3-4 replicates per location. The values are BLUPs for the difference from the null in bushel/acre (bu/ac). The BN value is the yield in bu/ac for the null. Yield data (bushel/acre; bu/ac) for the 10 transgenic events are shown in FIGS. 5A-5C together with the bulk null control (BN). The significant positive yield differences are shown in bold, whereas the significant negative yield differences are shown in italics. Yield analysis was by ASREML (VSN International Ltd), and the values are BLUPs (Best Linear Unbiased Prediction) (Cullis, B. R et al (1998) Biometrics 54: 1-18, Gilmour, A. R. et al (2009). ASRemI User Guide 3.0, Gilmour, A. R., et al (1995) Biometrics 51: 1440-50).

Statistically significant improvements in yield between transgenic and non-transgenic (bulk Nulls) plants in these reduced or normal nitrogen fertility plots were used to assess the efficacy of transgene. As shown in FIG. 5A, multiple events of PHP52729 showed a significant increase in yield (˜5-12 bu/ac) in multiple locations under normal nitrogen conditions. In low nitrogen conditions, the yield was neutral or slightly reduced (FIG. 5B). Multi-location analyses across different N treatments also identified several transgenic events with significant yield (˜3-5.5 bu/ac) improvements over the bulk nulls (FIG. 5C).

### 2^{nd} Year Testing

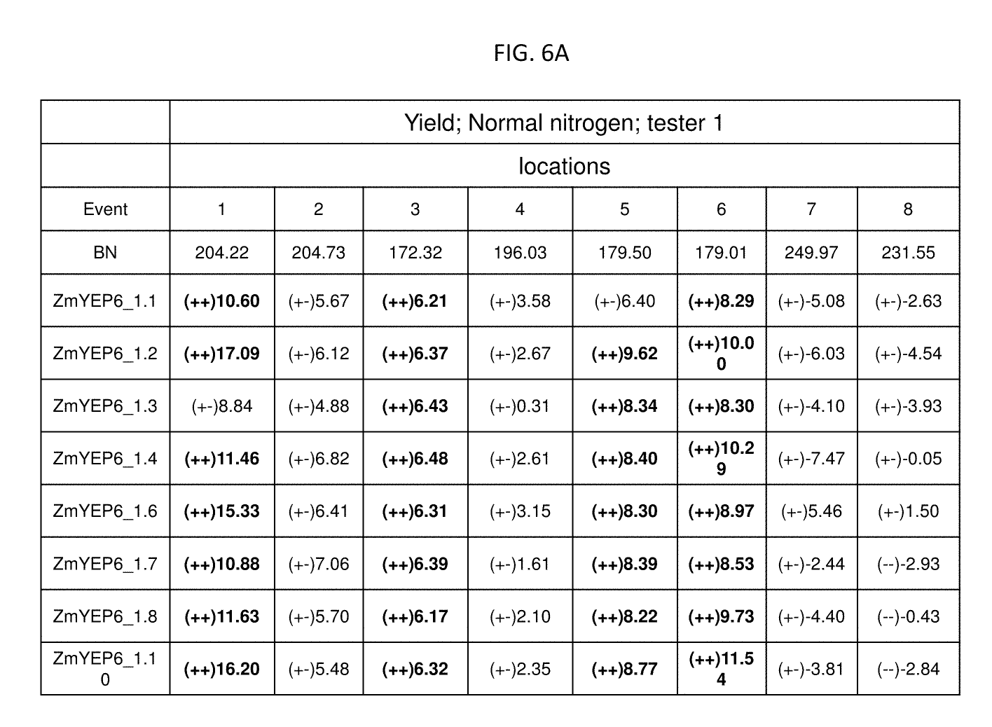

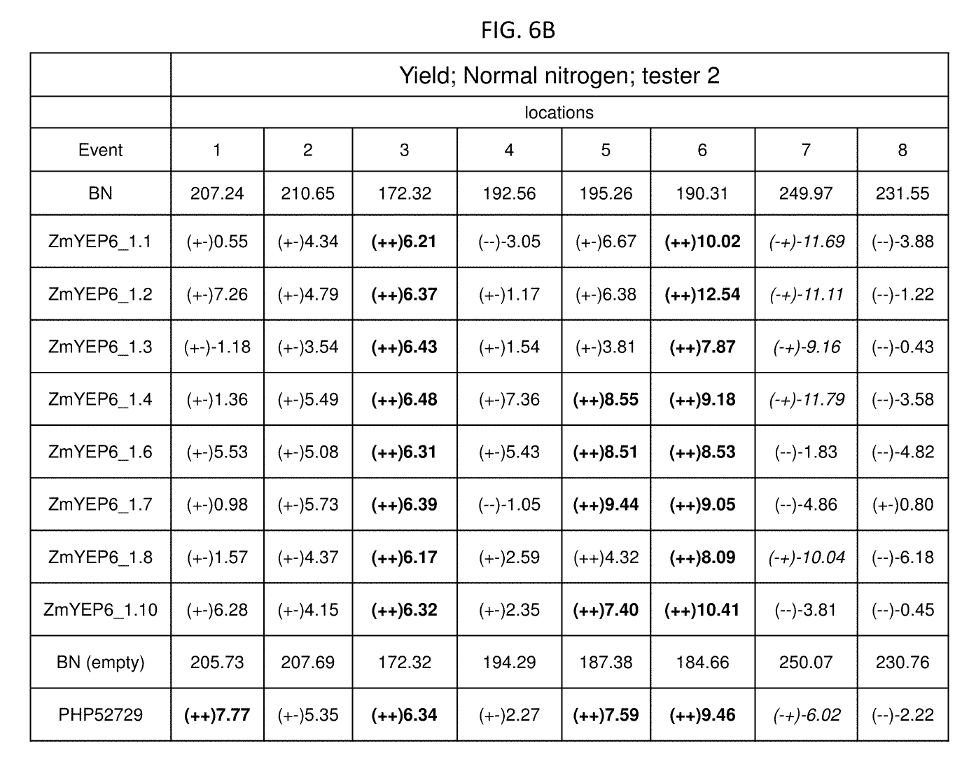

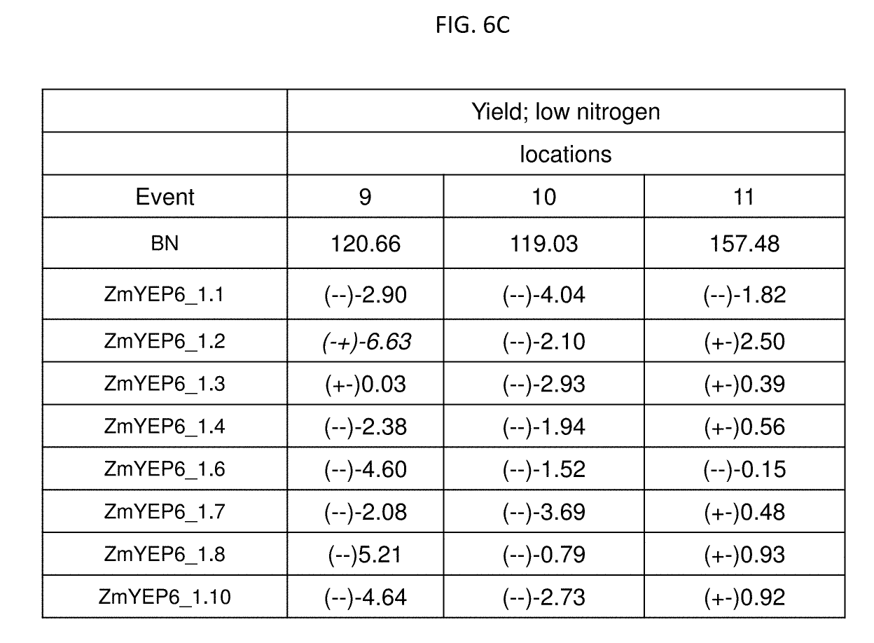

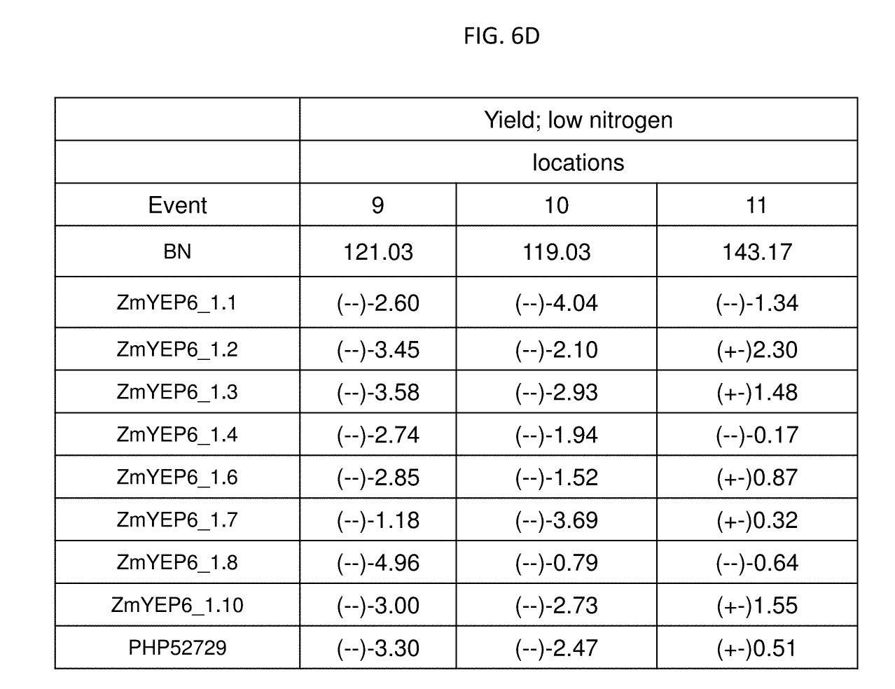

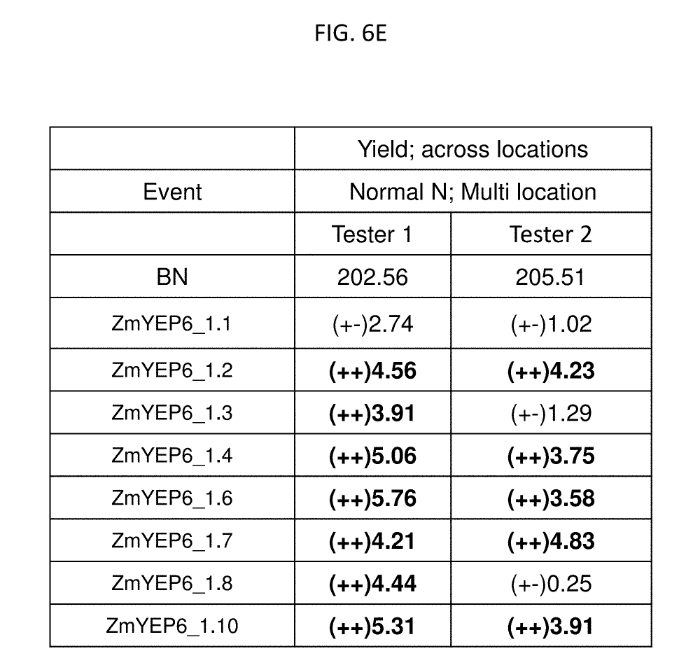

Events containing a single copy of the transgene with detectable transgene expression were advanced for field testing in a second subsequent year (year 2). Test crosses (hybrid seeds) were produced and tested in the field in multi-locations/replications experiments both in normal (8 locations; FIGS. 6A and 6B reflect crosses to tester 1 and tester 2, respectively) and low N (3 locations, FIGS. 6C and 6D) fields. Transgenic events were evaluated in normal nitrogen conditions and in low nitrogen conditions where yield is limited by reducing fertilizer application by 30% or more.

Yield data was collected in all locations, with 3-4 replicates per location. The values are BLUPs for the difference from the null in bushel/acre (bu/ac). The BN value is the yield in bu/ac for the null. Yield data (bushel/acre; bu/ac) for the 8 transgenic events is shown in FIGS. 6A-6E together with the bulk null control (BN).

The significant positive yield differences are shown in bold, whereas the significant negative yield differences are shown in italics. Yield analysis was by ASREML (VSN International Ltd), and the values are BLUPs (Best Linear Unbiased Prediction) (Cullis, B. R et al (1998) Biometrics 54: 1-18, Gilmour, A. R. et al (2009). ASRemI User Guide 3.0, Gilmour, A. R., et al (1995) Biometrics 51: 1440-50).

Statistically significant improvements in yield between transgenic and non-transgenic (bulk nulls) plants in the reduced or normal nitrogen fertility plots were used to assess the efficacy of the transgene. As shown in FIGS. 6A and 6B, multiple events of PHP52729 with tester 1 and tester 2 showed a significant increase in yield (˜6-13 bu/ac) in multiple locations under normal nitrogen conditions. FIG. 6B also shows a construct level average. In low nitrogen fields, the yield was neutral or slightly reduced (FIGS. 6C and 6D). FIG. 6D also shows the construct level average for yield. Multi-location analyses under normal N also showed that several transgenic events gave significant yield (˜3-5.5 bu/ac) improvements over the bulk nulls (FIG. 6E).

### Example 8

### Transgenic Events Showed a Significant Delay in Senescence

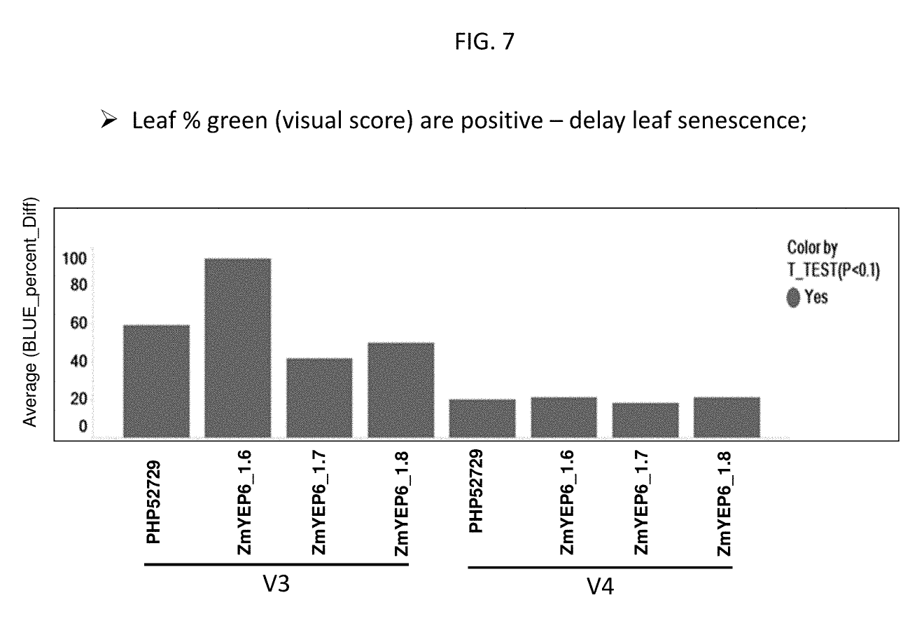

As ZmYEP6 was cloned by map based cloning for a leaf senescence phenotype, the transgenic events (for PHP52729) along with the null controls were also subjected to a senescence assay in a field pot study. Three events (inbreds) were grown in multiple replicates in field pots with drip irrigation at 2 and 8 mM nitrogen levels. Leaves V3 and V4 were scored for green area from when the plants were planted to the V6 stage of development. The data was statistically analyzed and clearly showed a significant delay of senescence in all transgenic events at both levels of nitrogen. In FIG. 7, a combined analysis across treatments is shown as % average difference in green area between transgenic events and nulls. The results clearly showed a delayed senescence in V3 and V4 both at the event and PHP levels.

### Example 9

### Staygreen Analysis of Maize Lines Transformed with PHP52729 Having Lower Expression of ZmYEP6 Gene

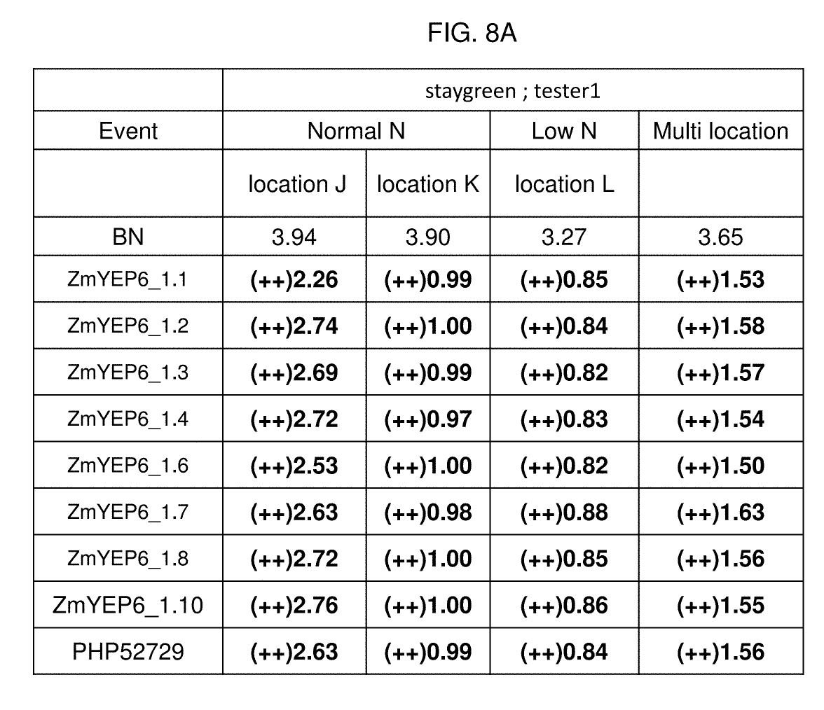

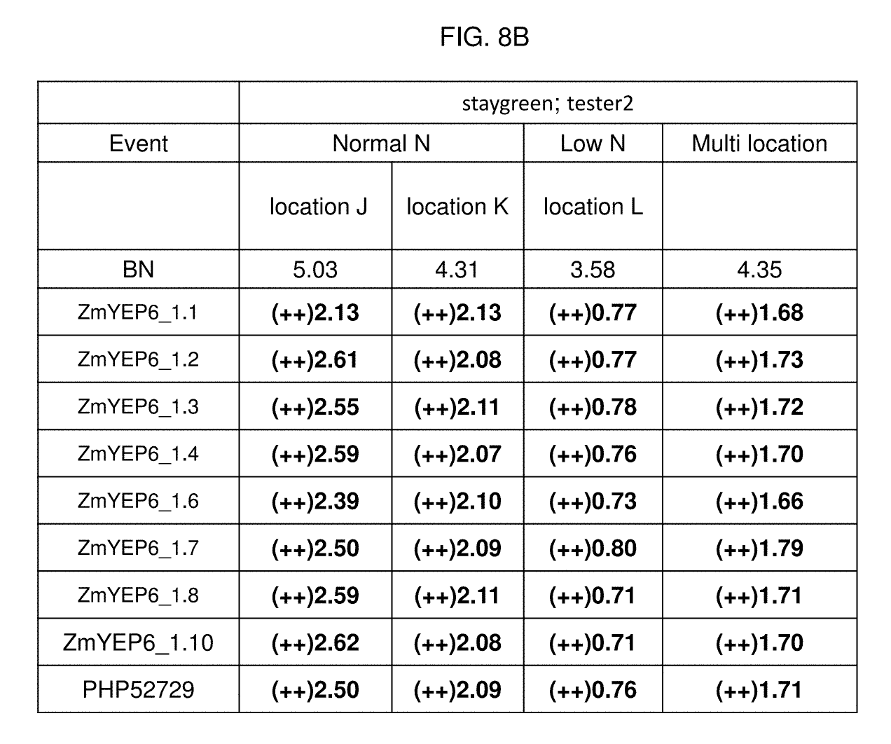

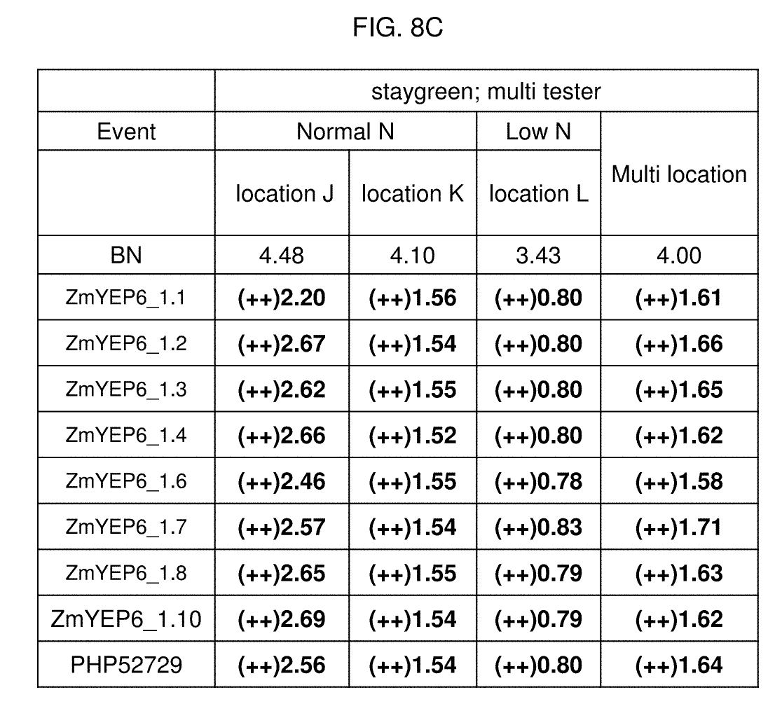

Eight transgenic events (hybrids) were field tested at one low-N location (“LN” location L) and at two locations where soil N levels were considered normal for maize production (“NN”; locations J and K). Two testers, tester 1 and tester 2, were used to assess potential transgene by genetic background interaction. FIG. 8A and FIG. 8B show the data for tester 1 and tester 2, respectively; and FIG. 8C shows the cumulative data for both testers.

The column “multilocation” in FIGS. 8A-8C shows the analysis for staygreen across all normal and low nitrogen locations.

Visual staygreen scores were collected in all locations, with 3-4 replicates per location. Scores ranged from 1-9 with “9” being a fully green canopy and “1” being completely senesced with no green. The scores were taken near the end of physiological maturity where optimal differences in canopy senescence can be observed.

Staygreen analysis was conducted using ASREML (Cullis, B. R et al (1998) Biometrics 54: 1-18, Gilmour, A. R. et al (2009). ASRemI User Guide 3.0, Gilmour, A. R., et al (1995) Biometrics 51: 1440-50). BLUEs (Best Linear Unbiased Estimates) were generated for both PHP52729 and the BN. The results reported in FIGS. 8A-8C are the difference of the transgenic BLUEs from the bulk null (BN) non-transgenic control BLUEs. Thus a positive value, indicated by “bold” in FIGS. 8A-8C represents a higher staygreen score than the BN. The cells with values in bold, represent differences that are significant at the P<0.10 level. In all genetic backgrounds and locations, the down regulation of the ZmYEP6 gene increased staygreen at the individual event level as well as at the construct level.

### Example 10

### Expression of ZmYEP6 in the High Protein (HP) and Low Protein (LP) Lines

The High Protein (HP) and Low Protein (LP) inbred lines described in Example 1 were tested for expression levels of the ZmYEP6 polypeptide. The RNAseq analysis in leaf showed that under low nitrogen conditions, low expression is correlated with staygreen (LP that shows staygreen phenotype shows lower expression levels of ZmYEP6), as shown below in Table 3.

TABLE 3
Expression Levels of the ZmYEP6 Polypeptide in Leaf Tissue
of HP and LP Inbred Lines under Low Nitrogen Conditions

| | | expression |
|---|---|---|
| line | DAP | level |
| LP | 0 | 25.1568 |
| HP | 0 | 61.1152 |
| LP | 16 | 10.2651 |
| HP | 16 | 195.09 |
| LP | 24 | 19.5039 |
| HP | 24 | 311.99 |

### Example 11

### Endogenous ZmYEP6 Expression is Induced During Senescence

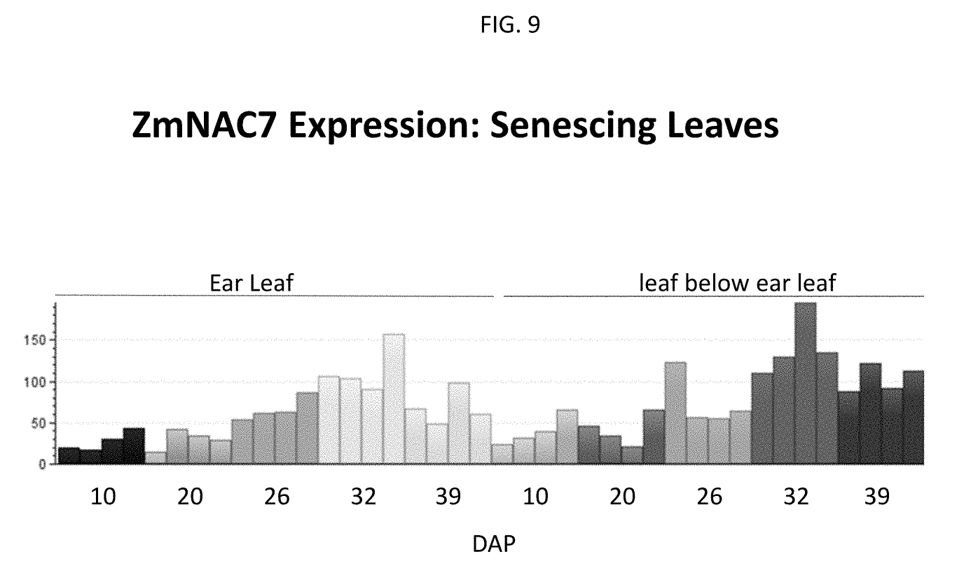

Multi-year experiments in normal nitrogen fields using the B73 inbred line were conducted to examine senescence induced gene expression changes in field-grown maize. Both ear leaf and leaf below ear leaf were collected from multiple replications starting about 10 days after pollination (DAP) till around 40 DAP. These samples were subjected to RNAseq analyses (Haas and Cody (2010) Nat Biotech volume 28 (5)). As shown in FIG. 9, ZmYEP6 expression was induced (8-10 folds) during senescence (about 32 DAP), which suggests a role of this gene in senescence.

What is claimed is:

1. A plant in which expression of an endogenous YEP6 gene is reduced, when compared to a control plant, wherein the YEP6 gene encodes a YEP6 polypeptide and wherein the plant exhibits at least one phenotype selected from the group consisting of: increased yield, increased abiotic stress tolerance, increased staygreen, and increased biomass compared to the control plant.

2. (canceled)

3. The plant of claim 1, wherein the plant exhibits increased abiotic stress tolerance, and the abiotic stress is drought stress, low nitrogen stress, or both.

4. The plant of claim 1, wherein the endogenous YEP6 polypeptide comprises an amino acid sequence with at least 80% sequence identity to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98.

5. The plant of claim 1, wherein the plant exhibits the phenotype of increased yield and the phenotype is exhibited under non-stress conditions.

6. The plant of claim 1, wherein the plant exhibits the phenotype of increased yield and the phenotype is exhibited under stress conditions.

7. The plant of claim 1, wherein the plant exhibits the phenotype under drought stress conditions.

8. The plant of claim 1, wherein the plant is a monocot plant.

9. The plant of claim 8, wherein the monocot plant is a maize plant.

10. The plant of claim 1, wherein the reduction in expression of the endogenous YEP6 gene is caused by sense suppression, antisense suppression, miRNA suppression, ribozymes, or RNA interference.

11. The plant of claim 1, wherein the reduction in expression of the endogenous YEP6 gene is caused by a mutation in the endogenous YEP6 gene.

12. The plant of claim 11, wherein the mutation in the endogenous YEP6 gene is caused by insertional mutagenesis.

13. (canceled)

14. (canceled)

15. (canceled)

16. (canceled)

17. (canceled)

18. (canceled)

19. A method of making a plant in which expression of an endogenous YEP6 gene is reduced, when compared to a control plant, and wherein the plant exhibits at least one phenotype selected from the group consisting of: increased yield, increased abiotic stress tolerance, increased staygreen and increased biomass, compared to the control plant, the method comprising the steps of introducing into a plant a suppression DNA construct comprising a polynucleotide operably linked to a heterologous promoter, wherein the suppression DNA construct is effective for reducing expression of an endogenous YEP6 gene.

20. The method of claim 19, wherein the suppression DNA construct is selected from the group consisting of: sense suppression construct, antisense suppression construct, ribozyme construct, RNA interference construct and an miRNA construct.

21. The method of claim 20, wherein the suppression DNA construct is an RNA interference construct and the RNA interference construct comprises at least 100 contiguous nucleotides of SEQ ID NO:1, 3, 5, 7, 9, 11, 13, 15, 17, 19, 21, 23, 25, 27, 29, 31, 33, 35, 37, 39, 41, 43, 45, 47 or 49.

22. The method of claim 21, wherein the RNA interference construct comprises a polynucleotide sequence that has at least 90% sequence identity to SEQ ID NO:55.

23. (canceled)

24. (canceled)

25. (canceled)

26. (canceled)

27. A method of making a plant in which activity of an endogenous YEP6 polypeptide is reduced or expression of an endogenous YEP6 gene encoding the YEP6 polypeptide is reduced, when compared to a control plant, and wherein the plant exhibits at least one phenotype selected from the group consisting of: increased yield, increased staygreen, increased abiotic stress tolerance and increased biomass, compared to the control plant, wherein the method comprises the steps of introducing a mutation in an endogenous YEP6 gene, wherein the mutation is effective for reducing the activity of the endogenous YEP6 polypeptide or expression of the endogenous YEP6 gene, and wherein the YEP6 polypeptide comprises an amino acid sequence of at least 95% sequence identity, when compared to SEQ ID NO:2, 4, 6, 8, 10, 12, 14, 16, 18, 20, 22, 24, 26, 28, 30, 32, 34, 36, 38, 40, 42, 44, 46, 48, 50, 57-97 or 98.

28. The method of claim 27, wherein the plant is maize.

29. The plant obtained by the method in claim 27.

30. (canceled)

31. (canceled)

32. (canceled)

33. (canceled)

34. (canceled)

35. (canceled)

36. (canceled)

37. (canceled)

38. (canceled)

39. (canceled)

40. A method of identifying one or more trait loci or a gene controlling such trait loci, the method comprising:
(a) developing a breeding population of maize plants, wherein the breeding population is generated by crossing a first maize inbred line characterized as a high protein line with a second maize inbred line characterized as a low protein line;
(b) selecting a plurality of progeny maize plants based on at least one phenotype of interest selected from the group consisting of delayed senescence, increased nitrogen use efficiency, increased yield, increased abiotic stress tolerance, increased staygreen, and increased biomass;
(c) performing marker analysis for the one or more phenotypes identified in the progeny of plants; and
(d) identifying the trait loci or the gene controlling the trait loci.


SEQUENCE LISTING (98 sequences)

SEQ ID NO: 1 - DNA, 1017 bp, Zea mays
atgtcgatgagcttcttgagcatggtggaggcggagctgccgccggggttccggttccacccgagggacgacgagctcatctgcgactacctcgcgcccaagctcggcgccaagcccggcttctccggctgccgcccgcccatggtcgacgtcgacctcaacaaggtcgagccatgggacctccccgtggcggcgtcggtggggccgcgggagtggtacttcttcagcctcaaggaccgcaagtacgcgacggggcagcggacgaaccgggccacggtgtccgggtactggaaggcgacggggaaggaccgacccgtggtggcggcgcggcgaggcgcgctggtggggatgcgcaagacgctcgtgttctaccaggggagggcgcccaagggcaggaagacggagtgggtgatgcacgagtacaggatggagccagctgctcctcttcttgatcaccaaccctcctcatccaactccaaggatgaagattgggtgctgtgcagagtcatctgcaagaagaaactggcagcaggaggccgcgcaggagggggcagctcgaggagcctggtcgccagcaacggcggccgcgagaccgcgccagccaccccgccgccgccgccgctgccacctcgcatggacacggacgccaccctagcacagctccaggccgccatgcacgccaccgccggcgcgctcgagcaggtgccctgcttctccagcttcaacaacaacactgccagctctagagctgctgccgcagcagcagcagcgcagccatgctacctgcccagcatggccacaggcggcagccacggcacgacgagctactacctagaccacgcgatgctgccgcctgagctgggtggctgcttcgatcctctccacggcgacaagaagctgctcaaggcggtgctgggccagctcggcggcgacgcggtggcgccgggcctgagcctgcagcacgagatggccgcgggcgctgtcgtcgcttcatccgcttggatgaatcacttctag

SEQ ID NO: 2 - protein, 338 aa, Zea mays
MSMSFLSMVEAELPPGFRFHPRDDELICDYLAPKLGAKPGFSGCRPPMVDVDLNKVEPWDLPVAASVGPREWYFFSLKDRKYATGQRTNRATVSGYWKATGKDRPVVAARRGALVGMRKTLVFYQGRAPKGRKTEWVMHEYRMEPAAPLLDHQPSSSNSKDEDWVLCRVICKKKLAAGGRAGGGSSRSLVASNGGRETAPATPPPPPLPPRMDTDATLAQLQAAMHATAGALEQVPCFSSFNNNTASSRAAAAAAAAQPCYLPSMATGGSHGTTSYYLDHAMLPPELGGCFDPLHGDKKLLKAVLGQLGGDAVAPGLSLQHEMAAGAVVASSAWMNHF

SEQ ID NO: 3 - DNA, 888 bp, Zea mays
atggggctgagggagatcgagtccacattgccgccggggttcaggttctatcccagcgacgaggagctggtgtgccactacctctacaagaaggtggccaacgagcgcgccgcgcaggggacgctggtggaggtcgacctgcacgcgcgagagccatgggagcttccagacgcggccaagctgacggcgagcgagtggtacttcttcagcttccgggaccgcaagtacgccacggggtcgcgcaccaaccgcgccaccaagacggggtactggaaggccaccggcaaggaccgcgaggtgcgcagcccggccacccgcgccgtcgtcggcatgaggaagacgctcgtcttctaccagggccgcgcacccaacggcgtcaagtcctgctgggtcatgcacgagttccgcctcgactcgccgcatacgccaccaaaggaggactgggtgctctgcagggtgttccagaagcggaaagacagcgagcaagacaacggcggcggctcctcctcgccgacgacctttgccggcgcgtcgtcgcagggggtcgttctagatctgccggacgaccagcccagcatgacgatggccggcgcgtacgtagcagcggaccaccagccgggctcttccgccgccgtcgggttcgcactgccgccgcatgcgcaggacaacggcctcggcgatggcggcctggacgcgttgctgatgaacggagcgacgatgtggcagtacagctcagcatcggccctcgccgatcacttcccgccggaggaagtgaccgccgcgcccatgatggggctaggctccaggggaggaggaggtgacgggtgcagcttcttctacgacagcggcttcgacgacatggggttcccgcagggatggatgggctga

SEQ ID NO: 4 - protein, 295 aa, Zea mays
MGLREIESTLPPGFRFYPSDEELVCHYLYKKVANERAAQGTLVEVDLHAREPWELPDAAKLTASEWYFFSFRDRKYATGSRTNRATKTGYWKATGKDREVRSPATRAVVGMRKTLVFYQGRAPNGVKSCWVMHEFRLDSPHTPPKEDWVLCRVFQKRKDSEQDNGGGSSSPTTFAGASSQGVVLDLPDDQPSMTMAGAYVAADHQPGSSAAVGFALPPHAQDNGLGDGGLDALLMNGATMWQYSSASALADHFPPEEVTAAPMMGLGSRGGGGDGCSFFYDSGFDDMGFPQGWMG

SEQ ID NO: 5 - DNA, 999 bp, Zea mays
atggggctgagggagatcgagtccacattgccgccggggttcaggttctatcccagcgacgaggagctggtgtgccactacctctacaagaaggtggccaacgagcgcgccgcgcaggggacgctggtggaggtcgacctgcacgcgcgagagccatgggagcttccagacgcggccaagctgacggcgagcgagtggtacttcttcagcttccgggaccgcaagtacgccacggggtcgcgcaccaaccgcgccaccaagacggggtactggaaggccaccggcaaggaccgcgaggtgcgcagcccggccacccgcgccgtcgtcgccatgaggaagacgctcgtcttctaccagggacgcgcacccaacggcgtcaagtcctgctgggtcatgcacgagttccgcctcgactcgccgcatacgccaccaaaggtatatatatacatacatatatgctactatgtgtgtgtgttgttgctgaccatcttggcccggccggtgtgcacgatcgacgttcctgtatgcgtgtggttgcaggaggactgggtgctctgcagggtgttccagaagcggaaagacagcgagcaagacaacggcggcggctcctcctcgccgccgacctttgccggcgcgtcgtcgcagggggtcgttctagatctgccggacgaccagcagcccagcatgacgatggccggcgcgtacgtagcagtggaccaccaccagccaggctcttccgccgtcgggttcgcactgccgccgcatgcgcaggacaacggcctcggcgatggcggcctggacgcgttgctgatgaacggagcgaccatgtggcagtacagctcatcggccctcgccgaccacttcccgccggaggaagtgaccgccgcgcccatgatggggctaggctccaggggaggaggtgacgggtgcagcttcttctacgacagcggcttcgacgacatggcgaacatggggttcccgcagggatggatgggctga

SEQ ID NO: 6 - protein, 332 aa, Zea mays
MGLREIESTLPPGFRFYPSDEELVCHYLYKKVANERAAQGTLVEVDLHAREPWELPDAAKLTASEWYFFSFRDRKYATGSRTNRATKTGYWKATGKDREVRSPATRAVVAMRKTLVFYQGRAPNGVKSCWVMHEFRLDSPHTPPKVYIYIHICYYVCVLLLTILARPVCTIDVPVCVWLQEDWVLCRVFQKRKDSEQDNGGGSSSPPTFAGASSQGVVLDLPDDQQPSMTMAGAYVAVDHHQPGSSAVGFALPPHAQDNGLGDGGLDALLMNGATMWQYSSSALADHFPPEEVTAAPMMGLGSRGGGDGCSFFYDSGFDDMANMGFPQGWMG

SEQ ID NO: 7 - DNA, 882 bp, Zea mays
atggggctgagggagattgagtcgacattgccgccggggttcaggttctatcccagcgacgaggagctggtatgccattacctctacaggaaggtggccaacgagcgctccgtgcaggggacgctggtggaggtcgacctgcacgcgcgggaaccatgggagcttccagacgcggccaagctgacggcgagcgagtggtacttcttcagcttcagggaccgcaagtacgcgaccgggccgcgcacgaaccgcgccaccaagacgggctactggaaggccaccggcaaggaccgcgaggtgcgcgacccggccgcccgcgccgtcgtcgtcggcatgcggaagacgctcgtcttctaccagggccgtgcccccagcggcgtcaagtcatgctgggtcatgcacgagttccgcctcgactcgccgcgtacgacgacgacgccaccgaaggaggactgggtgctgtgcagggtgttccagaagcggaaagtcgacggcagcgagcaagacaacggcgtctccccctcgccgccggtctttgccggcgcatcgcagtcgcagggggtcgtcctgccgccggaccagccctgcgtggtggacgcgtacgtcgtggtagaccagccgggctcttcttccgtcggactcgcgccgccgcaggagaacctcggtagcggcctggacgcgttgctgacgaacggagcgatgtggcagtacacctcgtcggtcttcggtcacttgttgccgcaggaggcgaccagctccccgccggtgatggacctaggctccaggggaggagaaggaggagactacgggtgcagcttcttctacgacagcagcttcgaggacatggccaacatcgggttccgacaagggtggatgggctga

SEQ ID NO: 8 - protein, 293 aa, Zea mays
MGLREIESTLPPGFRFYPSDEELVCHYLYRKVANERSVQGTLVEVDLHAREPWELPDAAKLTASEWYFFSFRDRKYATGPRTNRATKTGYWKATGKDREVRDPAARAVVVGMRKTLVFYQGRAPSGVKSCWVMHEFRLDSPRTTTTPPKEDWVLCRVFQKRKVDGSEQDNGVSPSPPVFAGASQSQGVVLPPDQPCVVDAYVVVDQPGSSSVGLAPPQENLGSGLDALLTNGAMWQYTSSVFGHLLPQEATSSPPVMDLGSRGGEGGDYGCSFFYDSSFEDMANIGFRQGWMG

SEQ ID NO: 9 - DNA, 885 bp, Zea mays
atggcattgagggagatcgagtcgacgctgccaccggggttcaggttctacccgagcgacgaggagctggtgtgccactacctccacaagaaggtggccaacgagcgcatcgcgcaggggacgctcgtcgaggtggacctgcacgcccgcgagccgtgggagctcccagaggtggcgaagctgacggccaccgagtggtacttcttcagcttccgggaccgcaagtacgccacggggtcgcgcaccaaccgcgccaccaggacgggctactggaaggccacgggcaaggaccgcgaggtgcgcagcagcagcagcgccaccgccgtcgtcgtcggcatgcgcaagacgctcgtcttctaccggggcagggcccccaacggcgtcaagtccggctgggtcatgcacgagttccgcctcgacaccccgcattcgccgccaagggaggactgggtgctgtgcagggtgttccagaagacgaaagccgccgacggcgacggcgatggcggcgcccaagacggcgactcctcgtccccgccggccgccttcaccggcccgtcacggccacgcgccgtgtcagagccgccggaccattccgcgccgccggcgggaggaggctactactacggcctcgcgttcgggccgcggcaggtggaggtggcgcagcagcagtgttacggcggcggcggcgccacggtcgccgaccaccaccacggcttcacgcgagacagcgggggcgctgcagggtttggcgcgacgtggggaggaggagtcgccggcggtgagtgcggggctggctacctcgatatggacggcgcctttgacgtcatgatggccggctgctttggcggtggagacatggatgagttccctcaagtgtggaggtga

SEQ ID NO: 10 - protein, 294 aa, Zea mays
MALREIESTLPPGFRFYPSDEELVCHYLHKKVANERIAQGTLVEVDLHAREPWELPEVAKLTATEWYFFSFRDRKYATGSRTNRATRTGYWKATGKDREVRSSSSATAVVVGMRKTLVFYRGRAPNGVKSGWVMHEFRLDTPHSPPREDWVLCRVFQKTKAADGDGDGGAQDGDSSSPPAAFTGPSRPRAVSEPPDHSAPPAGGGYYYGLAFGPRQVEVAQQQCYGGGGATVADHHHGFTRDSGGAAGFGATWGGGVAGGECGAGYLDMDGAFDVMMAGCFGGGDMDEFPQVWR

SEQ ID NO: 11 - DNA, 897 bp, Zea mays
atggggctgcgggacatcgagctcacgctgccgccggggttccggttctaccccagcgacgaggagctggtgtgccactacctgcacggcaaggtggccaacgagcagctcgcaggcgccggcgcagccatggtggaggtggatctgcacacccacgagccgtgggagctccctgacgtggcgaagctgagcaccaacgactggtacttcttcagcttccgcgaccgcaagtacgcgacggggcagcgcgccaaccgcgccaccaggtcgggctactggaaggccaccggcaaggaccgcgccatccacgaccccaggtccgccatcgtcgtcggcatgcgcaagaccctcgtcttctaccgcggccgcgcgcccaacggcgtcaagaccagctgggtgatgcacgagttccggatggtggaggacccccacgccccgcccaaggaggactgggttctatgcagggttttctacaagacgaaggccgacgacgccacggccgacagcgagcaagacgccgtgcgtatgcctcgcggcggcggcggcagcgctgatccgagctgctactcgcctcctccgttccccgcggcgctcggcggcagccaccaccaccaccaccatctgccgccgccgccgccgtcttcagaccgccgccacggcgccggctcccccgacgacgacttccccggaggcatggcgctgctgcagcacagcagcggcatgttcgacttccacggcggccagcctcgccctcacgacggcgtcgtcctcgctgggccggctgctgcagcggcggcgccggcgtcaagagacggcggcgatcagtgtggcagcggcgcgctcattgacctaggactggacgagcactacacctacaacagcctgctgcagatgtga

SEQ ID NO: 12 - protein, 298 aa, Zea mays
MGLRDIELTLPPGFRFYPSDEELVCHYLHGKVANEQLAGAGAAMVEVDLHTHEPWELPDVAKLSTNDWYFFSFRDRKYATGQRANRATRSGYWKATGKDRAIHDPRSAIVVGMRKTLVFYRGRAPNGVKTSWVMHEFRMVEDPHAPPKEDWVLCRVFYKTKADDATADSEQDAVRMPRGGGGSADPSCYSPPPFPAALGGSHHHHHHLPPPPPSSDRRHGAGSPDDDFPGGMALLQHSSGMFDFHGGQPRPHDGVVLAGPAAAAAAPASRDGGDQCGSGALIDLGLDEHYTYNSLLQM

SEQ ID NO: 13 - DNA, 891 bp, Zea mays
atggggctgcgcgacatcgagctgacgctgccgccggggttccggttctaccccagcgacgaggagctggtgtgccactacctgcacggcaaggtggccaacgagcggctcgccggcgccggcgcggccatggtggaggtggatctgcacacccacgagccgtgggagctccctgacgtggcgaagctgagcacgaacgagtggtacttcttcagcttccgcgaccgcaagtacgccacggggctgcgcaccaaccgcgccaccaagtccggctactggaaggccaccggcaaggaccgcgtgatccacaccccctgcagcaggcccgccgccggcggcggccaccaccgcgccgtcgtcggcatgcgcaagaccctcgtcttctaccgcggccgcgcccccaacggcgtcaagaccagctgggtgatgcacgagttccggatggagaacccccacaccccgcccaaggaggattgggtcctgtgcagggtcttctacaagaagaaggcagacgccatggactatgctgactgcagcgaccaggacgccatcgccgtgcatgtgcctcgcggcagcgctgacccgggctactgctactcgcctcctccgttccccgcgctcggcggcagccaccaccacggcggctccctcaacgacgacttccacggcggcatggcgctgcttcagcagcagcagcacaacggcgttttcgacttccacggcggccagcctcaccctcacggcggcggcggcggcctccttgccgggccgccggctacagccgtggggtcaagagacggcggcgatcagtgtggcagcggcgtgctcatggacctaggactcgacgaccactacgcctacaacagctacaacagcctgctgcagatgtga

SEQ ID NO: 14 - protein, 296 aa, Zea mays
MGLRDIELTLPPGFRFYPSDEELVCHYLHGKVANERLAGAGAAMVEVDLHTHEPWELPDVAKLSTNEWYFFSFRDRKYATGLRTNRATKSGYWKATGKDRVIHTPCSRPAAGGGHHRAVVGMRKTLVFYRGRAPNGVKTSWVMHEFRMENPHTPPKEDWVLCRVFYKKKADAMDYADCSDQDAIAVHVPRGSADPGYCYSPPPFPALGGSHHHGGSLNDDFHGGMALLQQQQHNGVFDFHGGQPHPHGGGGGLLAGPPATAVGSRDGGDQCGSGVLMDLGLDDHYAYNSYNSLLQM

SEQ ID NO: 15 - DNA, 849 bp, Zea mays
atgagctcgtcgatcagcatgatggaggcgagaatgcctccggggttcaggttccaccccagggatgacgagctcgtgatggactacctcctacaaaagctctccggccatggccaccatgccggcgctgccatcgtcgtcgacgtcgacctcaacaagtgcgagccatgggacctcccagattccgcgtgcgtgggtgggaaggagtggtacttcttcagcctgcgcgaccgcaagtacgcgacggggcagcgcactaaccgcgccacgcactccggctactggaaggccaccggcaaggaccgcgccgtcgtcgccggcggcgaggtggcggtggggatgcgcaagacgctggtcttctaccggggccgcgcgcccagggggaagaagacggagtgggtcatgcacgagttccgcctccacccccatgccgcgccgtgcctgctagcagcagctgcgccaaacaaggaggactgggtgctatgcagggtgttctacaagagcagaacaaccagcccaaggccagaatctgaagacgcccgggacggcaccccatcagctgaaccgcaactgccggctgccctgccgcttgcgcccctcgctgacacctacgccgccccaacggttgcagagcaggtgtactgcttctccggtctgccggcactgccattcagaagaccagtgagccttggggaccttctggagttcgacgcctccgagaaggaatctgtcacgacagcgatgaccagtgtctcgaacaacactagttcagtgctggagctggctccaaattgcaactggaaccaggaaaatggcatgtcacggatgtggagcccccttgggatatga

SEQ ID NO: 16 - protein, 282 aa, Zea mays
MSSSISMMEARMPPGFRFHPRDDELVMDYLLQKLSGHGHHAGAAIVVDVDLNKCEPWDLPDSACVGGKEWYFFSLRDRKYATGQRTNRATHSGYWKATGKDRAVVAGGEVAVGMRKTLVFYRGRAPRGKKTEWVMHEFRLHPHAAPCLLAAAAPNKEDWVLCRVFYKSRTTSPRPESEDARDGTPSAEPQLPAALPLAPLADTYAAPTVAEQVYCFSGLPALPFRRPVSLGDLLEFDASEKESVTTAMTSVSNNTSSVLELAPNCNWNQENGMSRMWSPLGI

SEQ ID NO: 17 - DNA, 918 bp, Zea mays
atgatggccatcaagtcgctgagcatggtggaggccagcctgcctccggggttcaggttccacccgcgcgacgacgagctcgtgctggactacctggccaagaagctcggcggcggcggaggccctgtggtggtgagcatctacggctgccccaccatggtcgacgtcgatctcaacaagtgcgagccctgggaccttcccgacatcgcgtgcattggtggaaaagagtggtatttctacagccttagagatagaaagtatgccactggccagcgtacaaacagagcaactgattcgggatactggaaggccacagggaaagaccgtccaataagccggaaagggttacttgttggtatgcgcaaaacccttgtgttttatcaaggtagagccccaaagggaaagaagaccgagtgggttatgcatgagtttcgcatggaagggcgagatgatcccatgaaattacctttcaaggaggactgggtcttgtgtagagttttctacaagagtagggcaacagttgcaaagccgcccacagagagcagcagcttcaatattgatgcagccacaacttcattgcctccccttattgacaacaacttcaatatctcctttgaccagcctggctcatcatcagtgcagaacctagagggttatgagcaagtgccctgcttctccagtaacccctctcagcagccatcgtcgtcgatgaacgccgcccggctgccgccgtctgccgccatggctgatccggagcagcagatggggaagtcaataatcaaggatgttctcatgagccagtttagcaggttcgaaggcagcgtcaagagggaggcgcctccaagcaatttttctcaggatgggtttgagtacttagctgagagtggcttcacgcagatgtggaattcgttcaattag

SEQ ID NO: 18 - protein, 305 aa, Zea mays
MMAIKSLSMVEASLPPGFRFHPRDDELVLDYLAKKLGGGGGPVVVSIYGCPTMVDVDLNKCEPWDLPDIACIGGKEWYFYSLRDRKYATGQRTNRATDSGYWKATGKDRPISRKGLLVGMRKTLVFYQGRAPKGKKTEWVMHEFRMEGRDDPMKLPFKEDWVLCRVFYKSRATVAKPPTESSSFNIDAATTSLPPLIDNNFNISFDQPGSSSVQNLEGYEQVPCFSSNPSQQPSSSMNAARLPPSAAMADPEQQMGKSIIKDVLMSQFSRFEGSVKREAPPSNFSQDGFEYLAESGFTQMWNSFN

SEQ ID NO: 19 - DNA, 954 bp, Zea mays
atgagcttgatcagcatgatggaggcgcggctgccgccggggttccggttccacccgagggacgacgagctcgtgctcgactacctctgccgcaagctctccggcaaaggcggcggcggagcgtacggcggcatcgccatggtcgacgtcgacctcaacaagtgcgagccgtgggatcttccagacgaggcgtgcgtgggcggccgcgagtggtacttcttcagcctgcacgaccgcaagtacgccacggggcagcggaccaaccgcgccacgcgcaccgggtactggaaggccacgggcaaggaccgccccatctccgtctccggccgccgcggggccggcgacaccgccgcgctggtcgggatgcgcaagacgctggtgttctaccagggcagggcgccccgcgggagcaagaccgagtgggtcatgcacgagttccgcgtggacggcccgcccgttgccgaccgccccggctcacctctcctccagctccagctccaggaggattgggtcctgtgcagggtgttctacaagagccgaactgccagcacaagaccagcagcaggccccgacgaggccgggccgctgtccagccagctgatcggcggcctgccgatgccgcgaatggccgcccctgctgacgccgcctacctgtccttcgacgtcacacctgccgctggcggctactaccaccaccaagactccggccccgcggacgcgcgccaccacctgccgccgccgccggcgcagcctttcagcaggagtagcctgtccagcttgcgggacttgctcagcagcatggttgaaggcagcgacgccgccgccgccgttcgggagacggagctccacctgcagctggagggctggaccgaggcggcctacgcgcagcagcagggcggcgccatgccggcgcacccgcagcagacgtggagcccgtttctgagctcgggatga

SEQ ID NO: 20 - protein, 317 aa, Zea mays
MSLISMMEARLPPGFRFHPRDDELVLDYLCRKLSGKGGGGAYGGIAMVDVDLNKCEPWDLPDEACVGGREWYFFSLHDRKYATGQRTNRATRTGYWKATGKDRPISVSGRRGAGDTAALVGMRKTLVFYQGRAPRGSKTEWVMHEFRVDGPPVADRPGSPLLQLQLQEDWVLCRVFYKSRTASTRPAAGPDEAGPLSSQLIGGLPMPRMAAPADAAYLSFDVTPAAGGYYHHQDSGPADARHHLPPPPAQPFSRSSLSSLRDLLSSMVEGSDAAAAVRETELHLQLEGWTEAAYAQQQGGAMPAHPQQTWSPFLSSG

SEQ ID NO: 21 - DNA, 1269 bp, Zea mays
atggtcttatggaggacaggaggagcatggtggtgctacctagcaagagctccccactataaagcgcccccacacaccataccacagctcagagcttcttctcatcatctggtagaaagaaagagtgagagtgaggttggcaagggtatagggttcttggtcgatcaaatacctttcccctcttggattctcatcttcctgcttcgttctcaccagatcgatctcaccacgtgcctgcgtagcaagccactctgtatgcaccatcaccaccaggaccaggccatgggcgacgcgctgtgggacctgctcggggaggagatggcagcggcgggcggggagcacgggctgcccccggggtttcggttccaccccaccgacgaggagctggtcaccttctacctcgccgccaaggtgttcaacggcgcctgctgcggcatcgacatcgccgaggtggacctcaaccggtgcgagccgtgggagctccccgacgcggcgcgcatgggggagcgcgagtggtacttcttcagcctccgcgaccgcaagtaccccacgggcctccgtaccaaccgcgccaccggcgccggatactggaaggccaccgggaaggaccgcgaggtgctcaacgccgccaccggcgcgctcctcggcatgaagaagacgctcgtcttctacaagggccgggcgccgcgcggcgagaagaccaagtgggtcctgcacgagtaccgcctcgacggcgacttcgccgccgctcgccgcccctgcaaggaggaatgggtcatctgcaggatactgcacaaggcaggcgaccagtatagcaagctgatgatggtgaagagcccctactacctgcccatggcgatggacccttccagcttctgcttccaggaggacccaaccgggcatcccctcccgaaccctagcggctgcacccccttccaccacggccacccccaccatagcatgcagccgccgcctccattgccgccgagcaaccatgctggcaaggccgtcttcaccggagcagcagcagcctgctgcatgcaacaagagccggcagacggcagcaacagcgccgtgctgcccatgccgccgttccctcccttcacccccatcgtcgccggcaagccggcggccccggcgccgccgccccaggttgtcaacgccggtccacaggagccaccgccacctacctggctggaggcctacctgcagcacactggtgggatcctttatgagatgggtccaactgcagcgcccaggggcgcgtga

SEQ ID NO: 22 - protein, 422 aa, Zea mays
MVLWRTGGAWWCYLARAPHYKAPPHTIPQLRASSHHLVERKSESEVGKGIGFLVDQIPFPSWILIFLLRSHQIDLTTCLRSKPLCMHHHHQDQAMGDALWDLLGEEMAAAGGEHGLPPGFRFHPTDEELVTFYLAAKVFNGACCGIDIAEVDLNRCEPWELPDAARMGEREWYFFSLRDRKYPTGLRTNRATGAGYWKATGKDREVLNAATGALLGMKKTLVFYKGRAPRGEKTKWVLHEYRLDGDFAAARRPCKEEWVICRILHKAGDQYSKLMMVKSPYYLPMAMDPSSFCFQEDPTGHPLPNPSGCTPFHHGHPHHSMQPPPPLPPSNHAGKAVFTGAAAACCMQQEPADGSNSAVLPMPPFPPFTPIVAGKPAAPAPPPQVVNAGPQEPPPPTWLEAYLQHTGGILYEMGPTAAPRGA

SEQ ID NO: 23 - DNA, 1074 bp, Zea mays
atggagcggttcggcctggacggcggcggtggcggtggcgagctgccgccggggttccggttccacccgacggacgaggagctcatcacctactacctcctccgcaaggccgtggacggcagcttctgcggccgcgccatcgcggagatcgacctgaacaagtgcgagccatgggagctcccggacaaggcgaagatgggggagaaggagtggtacttctacagcctccgcgaccgcaagtacccgacgggcctgcgcaccaaccgcgccacggtggccggctactggaaggccaccggcaaggaccgcgagatccgcagcggccgcaccggcgcgctggtgggcatgaagaagacgctcgtcttctaccgcggccgcgcccccaagggacagaagacgcactgggtcatgcacgagtaccgcctcgagggcgcctacgcctaccatttcctccccagctccacaagggacgagtgggtgatcgcgagggtgttccagaagcccggcgaggtcccacctgcagcccgcaagcaccgcctcggcgcccttagcagcactaccggcaccgccgcgggcgattcctgcttctcggactctacctcggcctccatcggcggcgcgtcgtcgtcatccacgcctggcccgctgttcgcgtcggcggccgccgccgtggccaatgccggcgccgccgacggcgacaccagctcctactgcggcggcgctgccaaccatggcaacctggtcaccggccgtgagctcgtgccctgcttctccactgccaccattaacggccccctagttgccgccgcgctcggcatcgggcagccgtacaacgcagccccgctgccgttcgagcagcagccgccgcctccggccttcctgccgagcctgcgttcccttcaggacaacctccagcttccaccgttcctctcagcaggcggtctgggcggcggcggggctctccactggctccccgccggcggcatggaggtcaaggtcgagggccgctcggcgccaccgcagatggctgtcggccccggccagctcgatggcgctttcggctggagcttctag

SEQ ID NO: 24 - protein, 357 aa, Zea mays
MERFGLDGGGGGGELPPGFRFHPTDEELITYYLLRKAVDGSFCGRAIAEIDLNKCEPWELPDKAKMGEKEWYFYSLRDRKYPTGLRTNRATVAGYWKATGKDREIRSGRTGALVGMKKTLVFYRGRAPKGQKTHWVMHEYRLEGAYAYHFLPSSTRDEWVIARVFQKPGEVPPAARKHRLGALSSTTGTAAGDSCFSDSTSASIGGASSSSTPGPLFASAAAAVANAGAADGDTSSYCGGAANHGNLVTGRELVPCFSTATINGPLVAAALGIGQPYNAAPLPFEQQPPPPAFLPSLRSLQDNLQLPPFLSAGGLGGGGALHWLPAGGMEVKVEGRSAPPQMAVGPGQLDGAFGWSF

SEQ ID NO: 25 - DNA, 1107 bp, Zea mays
atggagaggttgggcgtcggcgtcggcgtcggcgagctgccgccggggttccgcttccacccgacggacgaggagctgatcacctactacctcctctgcaaggccgtggacggcggcttctgcggcggccgcgccatcgcggagatcgacctgaacaagtgcgagccatgggagctcccggacaaggcgaagatgggggagaaggagtggtacttctactgcctccgcgaccgcaagtacccgacgggcctgcgcaccaaccgcgccacggcggccggctactggaaggccaccggcaaggaccgcgaggtccgcagcggccgcagcggcgcgctggtgggcatgaagaagacgctcgtcttctaccggggccgcgcccccaggggccagaagacgcgctgggtcatgcacgagtaccgcctcgacggcacctacgcctaccatttccttcccggctctacgagggacgagtgggtgatcgcgagggtgttccagaagccaggcgaggtcccatgcggccgcaagcaccgcctgggcggccccagcgccgccgccggcgagtcctgcttctcggactccaccacctcggcctccatcggcggcggcggcggaggaggagcgtccgcgtcgtctcgcccgctgctcaccgtcacggacacttcctcgccgtcgctgttcgtggccaacgcgaacgccgccgccagcaacaacaacggcaacccggtcaccgggcgagagctcgtgccctgcttctccactaccgccagtcccctggaagccgcggcgctcggcgtcgtcgggcacccgtacaacgcggccccgctgcgtctgggcttggacttcgaggcgccgtccccgggcttcgtcgtcccgaacctgcgttccctgcaagtgcaggacgacggcggcctgccgctgttcctctcggcagcagcaggcggcggcatgtcgtccgcgacgctgggaataatggggtcgctgggcgggtctctccactgcccgccccacgccggcatggatgtcgtcaaggtcgagggccgcgccgcgccgccgcagatggctgtcggccccggcctcctcgatggcgccttcgcctggggcttctag

SEQ ID NO: 26 - protein, 368 aa, Zea mays
MERLGVGVGVGELPPGFRFHPTDEELITYYLLCKAVDGGFCGGRAIAEIDLNKCEPWELPDKAKMGEKEWYFYCLRDRKYPTGLRTNRATAAGYWKATGKDREVRSGRSGALVGMKKTLVFYRGRAPRGQKTRWVMHEYRLDGTYAYHFLPGSTRDEWVIARVFQKPGEVPCGRKHRLGGPSAAAGESCFSDSTTSASIGGGGGGGASASSRPLLTVTDTSSPSLFVANANAAASNNNGNPVTGRELVPCFSTTASPLEAAALGVVGHPYNAAPLRLGLDFEAPSPGFVVPNLRSLQVQDDGGLPLFLSAAAGGGMSSATLGIMGSLGGSLHCPPHAGMDVVKVEGRAAPPQMAVGPGLLDGAFAWGF

SEQ ID NO: 27 - DNA, 1098 bp, Zea mays
atggagcaggagccgcaccggcccatggagctgcccccgggcttccgcttccacccgaccgacgaggagctcatcacgcactacctggcccgcaaggccgccgacgcccgcttcgccgcgctcgccgtcgccgaggccgacctcaacaagtgcgagccctgggacctgccatcgctggcgaggatgggggagaaggagtggtacttcttctgcctcaaggaccgcaagtacccgacggggctgaggaccaaccgggccacggaggccgggtactggaaggccacgggcaaggacagggacgtcttcaggggcaaggcgctcgtcggctccaagaagacgctcgtcttctacacggggagggcgcccaggggagagaagtccggatgggtcatgcacgagtaccgcctccacgccaagctccacggccacggccacggccacggccaaggcgcagccgtcgtcgtcgtccccaaggccgccgggaccaagaacgagtgggtgctgtgcagggtgttcaagaagagcctcgtcgtaggtggtgcagctgcagcagcagcaccaaccgcaggcaagaggggcggcacggagacgtcgtccaactccaagacgggcgacgtagcggccatctcccacctccctccgctgatggacgtgtccggctccggctccggcgccgccgccgcggcggcggcacacgtgacctgcttctccgacgcgctggagggccagttcttggaccagacgacgaccacgccgccgccagaagccgccgccgccgccacggacgacgacggccacctcggcgccctcgccgccgcctcctcctcggccttccagctgcccggcttcgcgcactactactacggcgggggggcgccgcacctgcaccagcaccacggcgccgccagcctggtgcagctcctggagggcagcgttccgccgtgcaataagggcggcgagcgggagcgggagcgggagcggctgagcgcgtcgcaggacacggggctcacctccgacgtcaaccccgagatctcctcctcctccggccaacgcttcgaccacgaccacgaccaccacctctgctgctggggctactga

SEQ ID NO: 28 - protein, 365 aa, Zea mays
MEQEPHRPMELPPGFRFHPTDEELITHYLARKAADARFAALAVAEADLNKCEPWDLPSLARMGEKEWYFFCLKDRKYPTGLRTNRATEAGYWKATGKDRDVFRGKALVGSKKTLVFYTGRAPRGEKSGWVMHEYRLHAKLHGHGHGHGQGAAVVVVPKAAGTKNEWVLCRVFKKSLVVGGAAAAAAPTAGKRGGTETSSNSKTGDVAAISHLPPLMDVSGSGSGAAAAAAAHVTCFSDALEGQFLDQTTTTPPPEAAAAATDDDGHLGALAAASSSAFQLPGFAHYYYGGGAPHLHQHHGAASLVQLLEGSVPPCNKGGERERERERLSASQDTGLTSDVNPEISSSSGQRFDHDHDHHLCCWGY

SEQ ID NO: 29 - DNA, 1080 bp, Zea mays
atggcggcggaccagcagccgcagctgcaggaggagatgaacgacgctgccggcggcggcctcaggctgcctccagggttccgcttccacccgagcgacttcgagattgtcagcttctacctcaccaacaaggtgctcaacacgcgcttcacctgcaccgccatcacggaggccgacctaaacaagattgagccatgggacctccctagcaaggcgaagatgggcgagaaagagtggtacttcttctaccagaaggaccgcaagtacccgacggggctgagggcgaaccgggccaccgaggccggttactggaaggcgacgggcaaggacaaggaggtctacaacgccgcggaaggggtggcggtactggtcggcatgaagaagacgctcgtcttctacaggggcagggctcccaggggtgacaagacaaactgggtcatgcacgagtacaggctcgaaggcagcggcaggctccccgccggcctcgcgtccgcaaccggctcagccgccgccaacgccgcggcggccttgaaagcttctgcttataagcaggatgagtgggtagtgtgtcgtgtgttccacaagaccactgggatcaagaagaccactgctgcaccggcgtaccaggtggccatggccggcgctgagatggatcagaatcagaacaacttcccgggcatccccttccccatgccgatgcaatttcccatgctgccagacttctccttggacccggtgcccccctactaccccaacgccgctggcgcggggatgtcgatgcttcctatggcagcaggtataggtggtggcgccggtgggttccagctcaacggcgccgccctgttcggcaatccgatggccgcgccgcagcccatgagcttctaccaccagatgggcgcggcggggacagcttgcgctggcggcttcgatgtttctgcgccggagagtaggccgtcctcgatggtgtcgcagaaggacgaccaggctaatggcgctgagatctcgtcgatgatgtccgtggccggcccagggcctgcgaccaccaccaccatagagatggatggcgtgtggaagtactga

SEQ ID NO: 30 - protein, 359 aa, Zea mays
MAADQQPQLQEEMNDAAGGGLRLPPGFRFHPSDFEIVSFYLTNKVLNTRFTCTAITEADLNKIEPWDLPSKAKMGEKEWYFFYQKDRKYPTGLRANRATEAGYWKATGKDKEVYNAAEGVAVLVGMKKTLVFYRGRAPRGDKTNWVMHEYRLEGSGRLPAGLASATGSAAANAAAALKASAYKQDEWVVCRVFHKTTGIKKTTAAPAYQVAMAGAEMDQNQNNFPGIPFPMPMQFPMLPDFSLDPVPPYYPNAAGAGMSMLPMAAGIGGGAGGFQLNGAALFGNPMAAPQPMSFYHQMGAAGTACAGGFDVSAPESRPSSMVSQKDDQANGAEISSMMSVAGPGPATTTTIEMDGVWKY

SEQ ID NO: 31 - DNA, 1176 bp, Zea mays
atggcggaccagcagcagccacagcagcagccgcaggagatggacgttgaccgtaccggtggcctcgaactgcctccagggttccgcttccacccgagcgactttgagattatcaacgactacctcacgaagaaggtgcacgacagggactacagctgcatcgccatcgcggacgccgacctaaacaagaccgagccatgggacctcccgaaagttgcaaagatgggcgagaaggagtggtacttcttctaccagaaggaccgcaagtacccgacggggctgagggcgaaccgggccactgaggcgggttattggaaggcgaccggcaaggacaaggaggtctacaacccctttgcagcggaagggctgctgctggtcggcatgaagaagacgctcgtgttctacaaaggcagggctcccaggggtgacaaaaccaactgggtgatgcacgagtacaggctcgaaggcagcggtaggctccctgctagtcctgcatccgcatccggctcagccaccaacatcgctgcggccatgatgaaagcttcagcttcggcttgcaaggatgagtgggtggtctgtcgtgtgttcaacaagaccaccgggatcaagaagacggctgcgccggcataccaggtggccatggccggtcctgagatggatcagaatcagaacaacattccggccatccccatccccatgccgctgcagctgccactgcccgtgcccatgcagatgcaatttcccatcctgccagattttgccatggacccggtggccccctactaccccaacccgaatgccggcgcggggatgatgccgcctatggcattggcaggtatgggtggcgccggcgggctccagatcaacggcgctctgttcggcaatccggtgcccgcgccgctgccgatgaacttctaccaccaccagatgggcatgggggcagcagctggccaggtggacatgggggcagcggctggccagatggacatgggagcagctggcgctggcgctggcggcttcgacgttgcagcgccggagagtaggccgtcctcgatggtgtcacagaaggacgaacaggctaatgccgccgagatctcgtcgatgatgtctgtgaccggcccagggtccgcgaccaccaccatagagatggatggcatatggaagtacaagtactga

SEQ ID NO: 32 - protein, 391 aa, Zea mays
MADQQQPQQQPQEMDVDRTGGLELPPGFRFHPSDFEIINDYLTKKVHDRDYSCIAIADADLNKTEPWDLPKVAKMGEKEWYFFYQKDRKYPTGLRANRATEAGYWKATGKDKEVYNPFAAEGLLLVGMKKTLVFYKGRAPRGDKTNWVMHEYRLEGSGRLPASPASASGSATNIAAAMMKASASACKDEWVVCRVFNKTTGIKKTAAPAYQVAMAGPEMDQNQNNIPAIPIPMPLQLPLPVPMQMQFPILPDFAMDPVAPYYPNPNAGAGMMPPMALAGMGGAGGLQINGALFGNPVPAPLPMNFYHHQMGMGAAAGQVDMGAAAGQMDMGAAGAGAGGFDVAAPESRPSSMVSQKDEQANAAEISSMMSVTGPGSATTTIEMDGIWKYKY

SEQ ID NO: 33 - DNA, 924 bp, Zea mays
atggcatcgtcgtcgcggctggatctcccgccggggttccgcttccaccccaccgacgaggaggtggtgtcgcactacctgacccacaaggcgctggacagccgcttctcctgcgtcgtcatcgccgacgccgacctgaacaagatcgagccgtgggatctcccaagcaaggctaagatgggcgagaaggagtggtacttcttctgccacaaggaccgcaagtacccgacggggatgcgcaccaaccgcgccaccgccagcgggtactggaaggccacgggcaaggacaaggagatcttccgcggcagtagtccccgccgcgtgctcgtcggcatgaagaagacgctcgtcttctacacgggccgcgcgccgcgcggaggcaagacgccgtgggtcatgcacgagtaccgcctcgagggcagcctgccgcccaacctccaccgcggggccaaggacgaatgggctgtgtgtaaggtgatcaacaaagacttggcgggcaaggctgggcagcaacaaatggcgccgccgcacgccgtgtccgtgggcatggagcgcagcgactcgctggccttcctcgacgacctggtgctcgacaacgccgacgacctgcccccgctcatcgactcgacaacgtacgccgcggcggggacgaccacgacgacgaacgacgacagcggcgggtaccaacaagccaccaaggcggagccgcagccgcatctgcccgcgccgagcaacagcccgtaccagcagcaggccatacggaggcactgcaaggcggaggcgccggcgccggcgatggttctgagcccgtcgcgcgagacgccgggcgsggacatgttccagctccagcatgtggacgagctgctccagctggacggsggcttcatggaggactactacaacatgaacatgtggaaggtctag

SEQ ID NO: 34 - protein, 307 aa, Zea mays
  misc_feature (277)..(277): Xaa can be any naturally occurring amino acid
MASSSRLDLPPGFRFHPTDEEVVSHYLTHKALDSRFSCVVIADADLNKIEPWDLPSKAKMGEKEWYFFCHKDRKYPTGMRTNRATASGYWKATGKDKEIFRGSSPRRVLVGMKKTLVFYTGRAPRGGKTPWVMHEYRLEGSLPPNLHRGAKDEWAVCKVINKDLAGKAGQQQMAPPHAVSVGMERSDSLAFLDDLVLDNADDLPPLIDSTTYAAAGTTTTTNDDSGGYQQATKAEPQPHLPAPSNSPYQQQAIRRHCKAEAPAPAMVLSPSRETPGXDMFQLQHVDELLQLDGGFMEDYYNMNMWKV

SEQ ID NO: 35 - DNA, 957 bp, Zea mays
atggcatcgtcgtcgcggctggatctcccgccggggttccgcttccaccccaccgacgaggaggtggtgtcgcactacctgacccacaaggcgctggacagccgcttctcctgcgtcgtcatcgccgacgccgacctcaacaagatcgagccgtgggatctcccaagcaaggctaagatgggcgagaaggagtggttcttcttctgccacaaggaccgcaagtacccgacggggatgcgcaccaaccgcgccaccgccagcgggtactggaaggccacggggaaggacaaggagatcttccgcggcagtagcagtagtccccgccgcgtgctcgtcggcatgaagaagacgctcgtcttctacacgggccgcgcgccgcgcggaggcaagacgccgtgggtcatgcacgagtaccgcctcgagggcagcctgccgcacaacctccaccgcggggccaaggacgaatgggctgtgtgtaaggtgatcaacaaagacttggcgggcaaggctgggcaacaacaaatggcgccgccgccgcacgccgtgtccgtgggcatggagcgcagcgactcgctggccttcctcgacgacctggtgctcgacaacgccgacgacctgcccccgctcgtcgactcgacaacgtacgccgccggctccctcttcgccgcggcggggacgaccacgacgacgaacgacgacagcggcgggtaccaacaagccaccaaggcggagccgcagccgcagctgcccgcgccgagcaacagcccgtaccagcaccagcagcaggccatacggaggcactgcaaggcggaggcgccggcgccggcgatggtgctgagcccgtcgcgcgagacgccgggcgcggacatgttccagctccagcatgtggacgagctgctccagctggacggcggcttcatggaggactactacaacatgaacatgtggaaggtctag

SEQ ID NO: 36 - protein, 318 aa, Zea mays
MASSSRLDLPPGFRFHPTDEEVVSHYLTHKALDSRFSCVVIADADLNKIEPWDLPSKAKMGEKEWFFFCHKDRKYPTGMRTNRATASGYWKATGKDKEIFRGSSSSPRRVLVGMKKTLVFYTGRAPRGGKTPWVMHEYRLEGSLPHNLHRGAKDEWAVCKVINKDLAGKAGQQQMAPPPHAVSVGMERSDSLAFLDDLVLDNADDLPPLVDSTTYAAGSLFAAAGTTTTTNDDSGGYQQATKAEPQPQLPAPSNSPYQHQQQAIRRHCKAEAPAPAMVLSPSRETPGADMFQLQHVDELLQLDGGFMEDYYNMNMWKV

SEQ ID NO: 37 - DNA, 1302 bp, Zea mays
atgccgccccagcccctccctccgctcctccagtccaagcccccttgccttctttctcagctcacctctctctttcacgcacgcctctctctctctctctctctctctctctctctctctctctctctctctctctctctctctctctctctctctctccttatctgtcctgaggcagccatttcctcttccgatcgtggagaggcttggagtggaaggaggctgctggttttcggcacggcggagatgtctgaggtgtcggtgataaaccaggcggaggtggaggacgcgggcgccgggcagctgatggacctgccgccgggcttccgcttccaccccaccgacgaggagatcatctcgcactacctcacccacaaggccctcgaccaccgcttcgtctccggcgtcatcggcgaggtcgacctcaacaggatcgagccatgggacctgccaggcagggccaagatgggggagaaggagtggtacttcttctgccacaaggatcgcaagtacccgacgggcacgcggaccaaccgcgccacggagaccggctactggaaggccaccggcaaggacaaggagatcttcaggggccgcggcgtcctcgtgggcatgaagaagacgctcgtcttctaccgcggccgcgcgccgcgcggggagaagaccggatgggtcatgcacgagttccgcctcgagggcaggcttccccagccgctcccgcgctccgccaaggacgagtgggccgtgtgcaaggtgttcaacaaggagctgcaggcggcgaggagcgagccattattggcggcggccggcgcggcggagctcgagcgcgtgggctcgctgggcttcctcaacgagctcctcgactccgcggagctgccggccctcgtcggagccgacgtggacgaggtgatcgacttcaagggccccgcccccgcgtccgttccggacgcgagctacctcccggtcaagatggaggagcacgcgctgctgcagatgcagcagtgccagtaccagccgccgcccccgatgttctacccgagccagtacttctctcttccggcgatgaactccggccacctccccccggcgatccggaggtactgcaaggcggagcagcaggtggtctcggcgcagacggcgtcggtgatcagcccgtcccgcgagaccgggctcagcaccgaccccaacgcggcgggcggctacgcggagatctcgtcggcggcgaccccgtcgtcgtcgcaccagttcctgcccgagctcgacgacccggccctcaacctcgccgacctctggaagtactga

SEQ ID NO: 38 - protein, 433 aa, Zea mays
MPPQPLPPLLQSKPPCLLSQLTSLFHARLSLSLSLSLSLSLSLSLSLSLSLSLLICPEAAISSSDRGEAWSGRRLLVFGTAEMSEVSVINQAEVEDAGAGQLMDLPPGFRFHPTDEEIISHYLTHKALDHRFVSGVIGEVDLNRIEPWDLPGRAKMGEKEWYFFCHKDRKYPTGTRTNRATETGYWKATGKDKEIFRGRGVLVGMKKTLVFYRGRAPRGEKTGWVMHEFRLEGRLPQPLPRSAKDEWAVCKVFNKELQAARSEPLLAAAGAAELERVGSLGFLNELLDSAELPALVGADVDEVIDFKGPAPASVPDASYLPVKMEEHALLQMQQCQYQPPPPMFYPSQYFSLPAMNSGHLPPAIRRYCKAEQQVVSAQTASVISPSRETGLSTDPNAAGGYAEISSAATPSSSHQFLPELDDPALNLADLWKY

SEQ ID NO: 39 - DNA, 1047 bp, Zea mays
atgtctgaggtgtcggtgataaaccaggcggaggtggaggatgcgggtgccgggcagctggacctgccgccggggttccgcttccaccccaccgacgaggagatcatctcgcactacctcgcccataaggccctcaaccaccgcttcgtctccggtgtcatcggcgaggtcgacctcaacaagtgcgagccatgggacctgccaggcagggccaagatgggggagaaggagtggtacttcttctgccacaaggatcgcaagtacccgacgggcacgcggaccaaccgcgccacggagaccggctactggaaggccaccggcaaggacaaggagatcttcaggggccgcggcgtcctcgtgggcatgaagaagacgctcgtcttctaccgcggccgcgcgccgcgcggggagaagaccggctgggtcatgcacgagttccgcctcgagggcaagcttcccgagcggctcccgcgctccgccaaggacgagtgggccgtgtgcaaggtgttcaacaaggagctggcggcgaggaccgagccaataatggcggcggccggcgcgggcgagctcgagcgcgtcggctcgctgggcttcctcagcgagctcctcgactccgccgagctgccggccctcatcggcgccgacgtcgacgaggtgatcgacttcaacggccccgcgtccacctccggcgcgccgggcacgagccacagccacctcccggtcaagatggaggagcacgcgctgctgcacatgcagtaccagccgccgccgcccccgacgtcctactactcgagccagtacttctctctgccggcgatgaactccggcgacgtccttcccccggcgatccggaggtactgcaaggcggagcagcaggtggtgtcggggcagacggcggcgtcggaggtcagcccgtcccgcgagaccgggctgagcgccgaccccaacgcggagatctcgtcggcggtgaccccgtcgtcgtcgcaccagttcctgcccgagttcgacgacccggtcctgaacctcgcggacctctggaagtactga

SEQ ID NO: 40 - protein, 348 aa, Zea mays
MSEVSVINQAEVEDAGAGQLDLPPGFRFHPTDEEIISHYLAHKALNHRFVSGVIGEVDLNKCEPWDLPGRAKMGEKEWYFFCHKDRKYPTGTRTNRATETGYWKATGKDKEIFRGRGVLVGMKKTLVFYRGRAPRGEKTGWVMHEFRLEGKLPERLPRSAKDEWAVCKVFNKELAARTEPIMAAAGAGELERVGSLGFLSELLDSAELPALIGADVDEVIDFNGPASTSGAPGTSHSHLPVKMEEHALLHMQYQPPPPPTSYYSSQYFSLPAMNSGDVLPPAIRRYCKAEQQVVSGQTAASEVSPSRETGLSADPNAEISSAVTPSSSHQFLPEFDDPVLNLADLWKY

SEQ ID NO: 41 - DNA, 969 bp, Zea mays
atggtggagctggagcctagtgtgaagtcggagcatggtggaggagtcggcctggtcctgcctcctggcttcaggttccatcccacagacgaagaggtcatcaccagctacctcctgcacaagttcctgaaccctagcttcgcgccgcacgccatcggggaggtggacctcaacaagtgcgagccatgggatctcccaagcaaggcgaagatgggggagaattccaaggagtggtacttcttctgccacaaggacatgaaataccccacgggcacgcgcgcgaaccgcgccaccaaggagggctactggaaggccacaggcaaggacagggagatcttcaagccagctgcagggcgtgaccgtgaccgcgagctagtggggatgaggaagacgctggtgttctacatgggcagggctccgcgcggcaccaagaccaactgggtgatgcacgagttccgcctcgagggcaagtccaggcacacctgcaacgacctacgcttcaatcccaaggatgaatgggtcgtgtgcaaggtgcaccacaaaggggccgaagaggccagcgccgccaagaagtccgccggcgggggcggcgaggagcactactactccaccgcaacgcccaacgtcagctccgtcgaaggcggcgacgagttcctcgtcgactccctgctggattactccagctacttcaactgcttcgcaaccggtggcttgcctctgcccagcacgacggccaactcagccgcagcacctgcaacgaacgacgacgactcctcctggagcatgcttcgtcacgcgccggcagaccagcaagcaatggtggggagctacagcttgcaccaccaggcaatgaaggcgaaaactgtaggcggcgtcacctccccgagtttctccgctggtttgccgagctcgccggtggcggactacgtgggaaacgatgcttctggctaccatacaaacaactattaa

SEQ ID NO: 42 - protein, 322 aa, Zea mays
MVELEPSVKSEHGGGVGLVLPPGFRFHPTDEEVITSYLLHKFLNPSFAPHAIGEVDLNKCEPWDLPSKAKMGENSKEWYFFCHKDMKYPTGTRANRATKEGYWKATGKDREIFKPAAGRDRDRELVGMRKTLVFYMGRAPRGTKTNWVMHEFRLEGKSRHTCNDLRFNPKDEWVVCKVHHKGAEEASAAKKSAGGGGEEHYYSTATPNVSSVEGGDEFLVDSLLDYSSYFNCFATGGLPLPSTTANSAAAPATNDDDSSWSMLRHAPADQQAMVGSYSLHHQAMKAKTVGGVTSPSFSAGLPSSPVADYVGNDASGYHTNNY

SEQ ID NO: 43 - DNA, 1116 bp, Zea mays
atggtggagcctagtgtgaaatcttcagagcatggtggaggaggaatcgacctgctgttcctgcctcctggcttcaggttccatcccaccgacgaggaggtcatcaccagctacctcctgcagaagctcctcaaccctagcttcgcgccgcacgccatcggggaggtggacctcaacaagtgcgaaccatgggatctcccaagcaaggcgaagatgggggaggaggattccaaggagtggtacttcttctgccgcaagggcatgaaatacccgacgggcacgcgggcgaaccgcgccaccaaggagggatactggaaggccacgggcaaggacagggagatcttcttcaagccagcagctgggcacgacgacgtcgaccgccgtgagcagctggtggggatgaagaagacgctggtgttctacacgggcagggctccaaggggcaccaagaccaactgggtgatgcacgagttccgcctcatcgagggcaagggcaagtactccaggcaccgcaccaccaacgacgacctactactacgcttcaaccccaaggatgaatgggtcgtgtgcaaggtgcaccacaaagggggccgacatgatgccagcgccgccaagaagggcggcggaggaggaggaggagaggagcagcaatactcgtccgccgccggaacgcccaacgtcagctccgtcgaggccggcggcggcggcgacgacgacgagttcctccttgactccgtgctgctggattactccagcagctgccacttcaactcctccggtcccaccgcttgcacctcatccagcaggagcaggagcatgccgccgccccgtcgtcacgcgccgccggcagaccagcagcaagcaataatggtgggagggagctactactacactagcttgcaccaccaccaccaggaaatggtcgcacccacgaggttcgccgccgccgctggtttgccgagtgcgtgtgcgtcggcggcgggcgccggagctgcgcagcgtagttcccagcagcatggtgtgctgcagcagcagaggcttcccgccgggaactacaactactacgacgacgggggaaactactacgctgcaggttaccgtacaagccagtactga

SEQ ID NO: 44 - protein, 371 aa, Zea mays
MVEPSVKSSEHGGGGIDLLFLPPGFRFHPTDEEVITSYLLQKLLNPSFAPHAIGEVDLNKCEPWDLPSKAKMGEEDSKEWYFFCRKGMKYPTGTRANRATKEGYWKATGKDREIFFKPAAGHDDVDRREQLVGMKKTLVFYTGRAPRGTKTNWVMHEFRLIEGKGKYSRHRTTNDDLLLRFNPKDEWVVCKVHHKGGRHDASAAKKGGGGGGGEEQQYSSAAGTPNVSSVEAGGGGDDDEFLLDSVLLDYSSSCHFNSSGPTACTSSSRSRSMPPPRRHAPPADQQQAIMVGGSYYYTSLHHHHQEMVAPTRFAAAAGLPSACASAAGAGAAQRSSQQHGVLQQQRLPAGNYNYYDDGGNYYAAGYRTSQY

SEQ ID NO: 45 - DNA, 1131 bp, Zea mays
atggaagcgtcagtaggagcagctggtggtggaggggggaagtcgaagaaggaggaggagagcctgccgccgggcttcaggttccacccgacggacgaggagctcatcacgtactacctgcggcagaagatcgccgacgccagcttcacggcgagggccatcgccgaggtcgacctcaacaagtgcgagccatgggatctcccggagaaagcgaagctgggagaaaaagagtggtatttcttcagcctgagggaccgcaagtacccaacgggtgtgcgaacaaaccgtgccactaacgctggttactggaagacaacggggaaagataaggaaatcttcaccggtcagctaccagccacgccagagctagtagggatgaagaaaaccctggtgttctacaaaggaagagctcctcggggcgagaagacaaactgggtcatgcatgagtatcgtctgcatcactcctctagatcagtccccaaatctaataaggacgagtgggtggtgtgccgggtgttcgccaagagcaccggcgctaagaagtacccgtccaacaacgcgcactcgcggttgcaccaccacccgtacgcgctggacatggtgccaccgctgctgcagcacgaccccttcgcgcgccaccacaactacccgtacatgacctcggccgacctggccgagctcgcgcgcttcgcccgcggcacgccggggctgcacccgcacatccagccggcgccgcaccccgggacgtcggcgtcggcgtacatgaaccccgccgccgccggggcgccaccgtcgttcgcgctctctggcggcctcagcctgaacctcggcgcctcgccggccatgccgccgtcgctgccgccgacagcattccacgcgacgatgtcgatggcgatgagcggacagacggcggcgccgagttgtgccggcaccggtaataaccatcgtcatcaggtggtggcgggtgagcaccagcagcagcagatggcggcggcggggctcggcggctgcgtgatcgtgcccggagcggacggagggttcgtcgccgacgcggccgccgggggtcggtaccagagcttggacgtggagcagctggtggagaggtactggcctgccggctaccaggtctag

SEQ ID NO: 46 - protein, 376 aa, Zea mays
MEASVGAAGGGGGKSKKEEESLPPGFRFHPTDEELITYYLRQKIADASFTARAIAEVDLNKCEPWDLPEKAKLGEKEWYFFSLRDRKYPTGVRTNRATNAGYWKTTGKDKEIFTGQLPATPELVGMKKTLVFYKGRAPRGEKTNWVMHEYRLHHSSRSVPKSNKDEWVVCRVFAKSTGAKKYPSNNAHSRLHHHPYALDMVPPLLQHDPFARHHNYPYMTSADLAELARFARGTPGLHPHIQPAPHPGTSASAYMNPAAAGAPPSFALSGGLSLNLGASPAMPPSLPPTAFHATMSMAMSGQTAAPSCAGTGNNHRHQVVAGEHQQQQMAAAGLGGCVIVPGADGGFVADAAAGGRYQSLDVEQLVERYWPAGYQV

SEQ ID NO: 47 - DNA, 1167 bp, Zea mays
atggaagcatcagcagcagcaggaggtgggagagagtcgaacaagaaggaggaggagagcttgccgccgggcttcaggttccacccgactgacgaggagctcatcacgtactacctgcggcggaagatcgccgacggcagattcacggcgagggccatcgccgaggtcgacctcaacaagtccgagccgtgggatctcccggagaaagcgaagctcggagaaaaagagtggtatttcttcagcctaagggaccggaagtacccaacaggcgtgcgaacgaaccgcgccactaacgctggttactggaagacaacggggaaagacaaggagatctacaccgccggtcatcagctacctgctgcagccaccacgccggagctagtagggatgaagaagaccctggtgttctacaaaggaagagctcctcggggtgagaagacgaactgggtcatgcatgagtatcgcctgcactccaaatcactccccaaatctaacaaggatgagtgggtggtgtgccgggtgttcgccaagagcgccgccgcaaagaagtacccgtccaacaacgcgcacgcgcacgcgcggtcatcgcaccaccacccgtacgcgctggacatgttcccacccctcctgcccacgctgctccagcatgaccccttcgtcgcgcgccgccaccaccaccaccacccgtacatggccccggcagacctggccgagctcgcgcgcttcgcccgcggcacgccggggctgcacccgcacatccagccgcaccccgggacgtcgtcgtcggcgccgtacatgaaccccgccgtggctgcgccaccgttcacgctctctggcggcggccgcctcaacctcaacctcggcgccacgccggccatgccgtcgtcgccgccagcactccacgcgatgtcgatggcgatgatgagcggccagacggagccgagctgtgccagcaccggtaggcaatatcatcaggtgatggcaggtgaacaccaccagcagaagcagatggcgacggcggctgcggcggggctcggcggctgcgtgatcgtgcccggcgcggacggagggttcggcgcggactcggcggccggggcccggtaccagggcttggacgtggagcagctggtggagaggtactggcctgccggcgggtaccaagtctag

SEQ ID NO: 48 - protein, 388 aa, Zea mays
MEASAAAGGGRESNKKEEESLPPGFRFHPTDEELITYYLRRKIADGRFTARAIAEVDLNKSEPWDLPEKAKLGEKEWYFFSLRDRKYPTGVRTNRATNAGYWKTTGKDKEIYTAGHQLPAAATTPELVGMKKTLVFYKGRAPRGEKTNWVMHEYRLHSKSLPKSNKDEWVVCRVFAKSAAAKKYPSNNAHAHARSSHHHPYALDMFPPLLPTLLQHDPFVARRHHHHHPYMAPADLAELARFARGTPGLHPHIQPHPGTSSSAPYMNPAVAAPPFTLSGGGRLNLNLGATPAMPSSPPALHAMSMAMMSGQTEPSCASTGRQYHQVMAGEHHQQKQMATAAAAGLGGCVIVPGADGGFGADSAAGARYQGLDVEQLVERYWPAGGYQV

SEQ ID NO: 49 - DNA, 1053 bp, Zea mays
atgcaaagggggcaagagcagcgggcgatggagctcgtcctgccgccgggcttccgcttcttcccgacggacgaagagctcctcacctgctacctcgccaggaaggccatggacggcagcttcaccacggcagccatccgcgaggtggacctctacaagacagagccatgggacctgccatgcgagcagcaggcggcggcggccggaggagacctgcacgagggctacttcttctgcaccaggggcagcaagtccccgtccggcattcgtgcccgccgcgccacacagctgggctactggaagtccacggggaaggacaagcccgtgcacagcaggtctggccgcctcgtcgtcgggacgaggaagacgctagtgttctaccgtggcagggcccccagaggggagaagactgactgggtgatgcacgagtactccatgggcgagaggaggagcagcgccctcctcagaggagcacagagtgagtgggtcatttgcagggtgttcacgaggaagcagcatccagtgatcagcaacgacaggaaactaccggaaatggaggaggctgccgtgcatgggcatggccaccgctctcctggccatctccttgccacggaggcagcagacgacggtttcgacagcgagcaggaatcggcgccaccggtcgtcgtcaccgagacccagcatacatcaggaagccacatcggcggtgcccaagccatggagggcgacgacgaccattatcagcaccgtcaaatagcccatgaggagctactgacgacgatgcaccatcaccacggttccagtcgcgtcgtctcgccagcatgttggctcaaccagcatgacgaccggctgggatcgcattattattgccccgcgttgccagtcatgcagtccgacgatgcggattactatctaccagagttgctggagtacagcggtccgctggacaccggcggtgaggaggactgccgtctccgtgctgagactcagttcaccgccgtgggctcctccgatcacatcgacgacgggctctactgggacattggcttttga

SEQ ID NO: 50 - protein, 350 aa, Zea mays
MQRGQEQRAMELVLPPGFRFFPTDEELLTCYLARKAMDGSFTTAAIREVDLYKTEPWDLPCEQQAAAAGGDLHEGYFFCTRGSKSPSGIRARRATQLGYWKSTGKDKPVHSRSGRLVVGTRKTLVFYRGRAPRGEKTDWVMHEYSMGERRSSALLRGAQSEWVICRVFTRKQHPVISNDRKLPEMEEAAVHGHGHRSPGHLLATEAADDGFDSEQESAPPVVVTETQHTSGSHIGGAQAMEGDDDHYQHRQIAHEELLTTMHHHHGSSRVVSPACWLNQHDDRLGSHYYCPALPVMQSDDADYYLPELLEYSGPLDTGGEEDCRLRAETQFTAVGSSDHIDDGLYWDIGF

SEQ ID NO: 51 - DNA, 21 bp, Artificial Sequence
  forward marker
ggtgatttgaaagggaaatgc

SEQ ID NO: 52 - DNA, 19 bp, Artificial Sequence
  reverse marker
gccttcgagctgctttagg

SEQ ID NO: 53 - DNA, 18 bp, Artificial Sequence
  forward marker_2
gcagccgacgaatcaacc

SEQ ID NO: 54 - DNA, 20 bp, Artificial Sequence
  reverse marker_2
cagtgaaagtcaggcagagc

SEQ ID NO: 55 - DNA, 310 bp, Artificial Sequence
  sequence used for RNAi of ZmYEP1
agtggtacttcttcagcctcaaggaccgcaagtatgcgacggggcagcggacgaaccgggccacggtgtccgggtactggaaggcgacggggaaggaccgacccgtggtggcggcgcggcgaggcgcgctggtggggatgcgcaagacgctcgtgttctaccaggggagggcgcccaagggcaggaagacggagtgggtgatgcacgagtacaggatggagccagctgctcctcttcttgatcaccaaccctcctcatccaactccaaggatgaagattgggtgctgtgcagagtcatctgcaagaagaa

SEQ ID NO: 56 - protein, 548 aa, Artificial Sequence
  consensus sequence
  X (1)..(548): any amino acid
MXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXLPPGFRFHPTDEELVTHYLARKVANXRXXXXXXFXXXXAIVEVDLNKCEPWDLPXXDKAKMGEXXXKEWYFFSLRDRKYPTGLRTNRATKAGYWKATGKDREIFXGXXXXXXXXXXXXXLVGMKKTLVFYRGRAPRGEKTNWVMHEFRLXXXXXXXXXXXXXXXXXXXXXXXXXXXXXXXXXXXXLXXXXSXKDEWVLCRVFQKSKXAXXXXAXXADSXXXXXXAXXXGGESXXXXXXXXPLXXXLDXXXXXQXXPLXAXAGADVXAXXXXXXXAAXGFXAXXXPXHAGAXALGAGXXXXXXXXLLXXXXXXXXXXXXXXXXXXXXXXXXXXXXMXGXXXXMFXXXXXXXLXXXXXXXXXXXXPAXALGAAXXXXXSXXGXXXXXXXYDGGFDXXXXXXXXXXXXXXXXXXXXXXXXXXXXXWXXXXXGX

SEQ ID NO: 57 - protein, 396 aa, Oryza sativa
MVESTTSLVKLEQDGGLFLPPGFRFHPTDAEVILSYLLQKLLNPSFTSLPIGEVDLNKCEPWDLPSKAKMGEKEWYFFSHKDMKYPTGMRTNRATKEGYWKATGKDREIFRQPAAVNTSSYGGSSNKKKQLVGMKKTLVFYMGRAPKGTKTNWVMHEFRLHANLHNHHPNLRLNPKDEWVVCKVFHKKQGDEAINNQQQQPQYAAVDQYSAETPNSGSSVVQAGDIDGGDDFFQLDDIIDPSIYFVSNSSNILSAPPNNNNAVYSVSASTTTTNTTAVSFQQQPNYYSLINKSSSSSSSNYSAPLQQHVSSWNITPGAGGAHGIGSSYYNLQQQQAAMVKALENVIAVPNFGTLLPSSNKLKGLSKSAMAGLTQQNPLGVPQYKIENYGDHYISRQ

SEQ ID NO: 58 - protein, 333 aa, Oryza sativa
MSMMSFLSMVEAELPPGFRFHPRDDELICDYLAPKVAGKVGFSGRRPPMVDVDLNKVEPWDLPEVASVGGKEWYFFSLRDRKYATGQRTNRATVSGYWKATGKDRVVARRGALVGMRKTLVFYQGRAPKGRKTEWVMHEYRMEGVHDQQASSFSSKEDWVLCRVICKRKSGGGATSKSRSLTTTTTTIVHDTSTPTSSPPLPPLMDTTLAQLQASMNTSSSSAIAAVAALEQVPCFSSFSNSIASNNNNSNSATVNAQQCYLPIVTGSNNNGMSYLDHGLPEFGSFLDTQSCDKKMLKAVLSQLNSIGGEVLPGLPPPSEMAAAVSSSWMNHF

SEQ ID NO: 59 - protein, 352 aa, Oryza sativa
MAMGMEGSGGGGSAKKKEESLPPGFRFHPTDEELITYYLRQKIADGGFTARAIAEVDLNKCEPWDLPEKAKMGEKEWYFFSLRDRKYPTGVRTNRATNAGYWKTTGKDKEIFTGQPPATPELVGMKKTLVFYKGRAPRGEKTNWVMHEYRLHSKSIPKSNKDEWVVCRIFAKTAGVKKYPSNNAHSRSHHPYTLDMVPPLLPALLQQDPFGRGHHPYMNPVDMAELSRFARGTPGLHPHIQPHPGYINPAAPFTLSGLNLNLGSSPAMPPPPPPPPQSILQAMSMPMNQPRSTTNQVMVTEQMIPGLANGVIPQGTDGGFTTDVVVGGTGIRYQNLDVEQLVERYWPGSYQM

SEQ ID NO: 60 - protein, 340 aa, Oryza sativa
MGDALWEMLGEEMAAAAAAAGEHGLPPGFRFHPTDEELVTFYLAAKVFNGACCGGVDIAEVDLNRCEPWELPEAARMGEKEWYFFSLRDRKYPTGLRTNRATGAGYWKATGKDREVVAAAAAGGALIGMKKTLVFYKGRAPRGEKTKWVLHEYRLDGDFAAARRSTKEEWVICRIFHKVGDQYSKLMMMKSPASYYLPVSHHHPSSIFHDLPPVPFPNPSLVPFHHDLPTSFHPPLLQHSHANSKNSSSNNGGFVFPNEPNTTNSSDNHISCNGAMAAAAAAAFPSFSCASTVTGKGGPPAQLGVNAGQQEPPPPTWMDAYLQHSGFIYEMGPPAVPRGA

SEQ ID NO: 61 - protein, 293 aa, Oryza sativa
MSFIGMVEARMPPGFRFHPRDDELVLDYLLHKLAAGGRGGGVYGGGGGVAIVDVDLNKCEPWDLPDAACVGGKEWYFFSLRDRKYATGHRTNRATRSGYWKATGKDRSITRRSSISSGEPSSSAAAAAVGMRKTLVFYRGRAPKGRKTEWVMHEFRLEPQPLHLKEDWVLCRVFYKTRQTIPSPSSEEAVTLPNELDLPATPSLPPLIDAYIAFDSAPTTTPSMVGSYEQVSCFSGLPALPMKGSISFGDLLAMDTSAEKKAIRVLHNSNTAKLELSPDWGQESGLSQMWNPQ

SEQ ID NO: 62 - protein, 343 aa, Oryza sativa
MEQHQGQAGMDLPPGFRFHPTDEELITHYLAKKVADARFAALAVAEADLNKCEPWDLPSLAKMGEKEWYFFCLKDRKYPTGLRTNRATESGYWKATGKDKDIFRRKALVGMKKTLVFYTGRAPKGEKSGWVMHEYRLHGKLHAAALGFLHGKPASSKNEWVLCRVFKKSLVEVGAAGGKKAAVVTMEMARGGSTSSSVADEIAMSSVVLPPLMDMSGAGAGAVDPATTAHVTCFSNALEGQFFNPTAVHGHGGGDSSPFMASFTQYGQLHHGVSLVQLLESCNGYGGLVDMAASGSQLQPAACGGERERLSASQDTGLTSDVNPEISSSSGQKFDHEAALWGY

SEQ ID NO: 63 - protein, 323 aa, Oryza sativa
MEEGLPPGFRFHPTDEELVTYYLARKVSDFGFATRAIADVDLNKCEPWDLPSKASMGEKEWYFFSMRDRKYPTGIRTNRATDSGYWKTTGKDKEIFHGGALAGMKKTLVFYRGRAPKGAKTSWVMHEYRLQSKFPYKPAKDEWVVCRVFKKLQCHLAKPRPPHDDVDGDGASPPEMVDASSLGELGELDVSSILLGGFAPPSGELCHGGGGGDGFGAHRLHVGAYMSWLQAAAAANQGMFQWPAATQAGLVGGTVFAAAHKAAGTMPFGGGCSQQQARDVGVSLANVGGGDALFGGAPLAKVDMECGEQAPQLDMDDSTWRAF

SEQ ID NO: 64 - protein, 358 aa, Oryza sativa
MSEVSVMAEVEETAAAAPLDLPPGFRFHPTDEEIVSHYLTPKALNHRFSSGVIGDVDLNKCEPWHLPAMAKMGEKEWYFFCHKDRKYPTGTRTNRATESGYWKATGKDKEIFRGRGILVGMKKTLVFYLGRAPRGEKTGWVMHEFRLEGKLPSQLPRSAKDQWAVCKVFNKELALAAKNGPMAVTEATADDAGIERVGSFSFLSDFIDPAELPPLMDPSFVADIDGVDDAKVSASTSGQAAIAAGFHVASQVMSYQQVKMEEPLPLPYLHQQPPRMLHSGQYFSLPAVHPGDLTPSAIRRYCKAEQVSGQTSALSASRDTGLSTDPNAAGCAEISSAPTSQPFPEFDDGILGLDDFWN

SEQ ID NO: 65 - protein, 374 aa, Oryza sativa
MRLARQQQQVVVAATMEHDVHHHRQMMQQQQQQEMDLPPGFRFHPTDEELITHYLLRKAADPAGFAARAVGEADLNKCEPWDLPSRATMGEKEWYFFCVKDRKYPTGLRTNRATESGYWKATGKDREIFRGKALVGMKKTLVFYTGRAPRGGKTGWVMHEYRIHGKHAAANSKQDQEWVLCRVFKKSLELAPAAAAAVGRRGAGAGTDVGPSSMPMADDVVGLAPCALPPLMDVSGGGGGAGTTSLSATAGAAAAPPPAHVTCFSNALEGQFLDTPYLLPAADPADHLAMSSASPFLEALQMQYVQDAAAAGGAGMVHELLMGGGWYCNKGERERLSGASQDTGLTSSEVNPGEISSSSRQQRMDHHDASLWAY

SEQ ID NO: 66 - protein, 359 aa, Oryza sativa
MVETSTSLVKLEQDGSLFLPPGFRFHPTDAEVILSYLLQKFLNPSFTSLPIGEVDLNKCEPWDLPSKAKMGEKEWYFFSHKDMKYPTGMRTNRATKEGYWKATGKDREIFNLQPTSYGGSSNNKNNKQLVGMKKTLVFYMGRAPKGTKTNWVMHEFRLHANLHNDNPNLRLNLKDEWVVCKVFHKKGDDREAINKQQAQAAAVDQYSAGTPNNGSSVEAGDDDDDLFQLDSIIDPSIYFSNSSAANILSAPPNMSNSVVAANYGASTTTTGTASAGSFQQQPNYCSLINKSISSSNVSSWNNMPPPPPVAEGGVHGIGSSYSLQHQAAMVKALRDVIRLPNPLGMPQYKLDDAYLWDSS

SEQ ID NO: 67 - protein, 358 aa, Oryza sativa
MAEEDDKKQKGPDVTVPSGYFFVPKPEQLIRDYLNHWITGRPSEELRDIVREADVYGSDPATLTEAHSAYGHDGKSWREKTTGASQQNIFTISRRGGFEGGGTWHNSQRRRVIEGYGDRQAFEYRAPGNKKTDWLMEEIASNLPAAITDEGIMVICKVYLSPRAKEATANEEERQETNVVPGPKRLREAEATGYDAPAPPQPDVGYSYSGGGETSQATASMDYCCSTTTHTADDTANAAYYHGDADAIKPDAYDGGDYGIGFNADGELVLCGNGHGGIGTQGQTPLAMQNTNGEMTLFSPMNGYGVGFNEEVRQEPQVEGEVEMDNFFNDLFVDFDGAGDLNPNPNGGGDSHGHILCE

SEQ ID NO: 68 - protein, 365 aa, Oryza sativa
MAEEDDKKQKGPDVTVPSGYFFVPKPEQLIRDYLNHWITGRPIEELRDIVREADVYGSDPATLTEAHRAYGHDGKSWYFLTVAKWKGGRGGAGTAGRLNRCVEGGGTWHNSQRRRVIEGYGDRQAFEYRAPGNKKTNWLMEEIASNLPAAITDEGIMVICKVYLSPRAKEATADEEERQETNVVPGPKRLREAEATGYDAPAPETPQPDVGCSYSGGGETSQATASMDYCCSTTTHTADDTANAAAYYYGDVDAIKPDAYDGGDYGIGINADGELVLCGNGHGGIGTQGQMPLAMQNTNGEMTLFSPMNGYGVGFNEEVRQEPQVGGEVEMNDFFNDLFVDFDGAGDPNPNPNEGGDSHGHILCE

SEQ ID NO: 69 - protein, 320 aa, Oryza sativa
MGEQQQQVERQPDLPPGFRFHPTDEEIITFYLAPKVVDSRGFCVAAIGEVDLNKCEPWDLPGKAKMNGEKEWYFYCQKDRKYPTGMRTNRATEAGYWKATGKDKEIFRDHHMLIGMKKTLVFYKGRAPKGDKTNWVMHEYRLADASPPPPPSSAEPPRQDDWAVCRIFHKSSGIKKPVPVAPHQVPAAANYQQQQQMAMASAGIIQVPMQMQMPSMSDQLQMLDDFSTTASLSLMAPPSYSTLPAGFPLQINSGAHPQQFVGNPSMYYHQQQQMDMAGGGFVVSEPSSLVVSPQDAADQNNNAADISSMACNMDAAIWKY

SEQ ID NO: 70 - protein, 333 aa, Oryza sativa
MGEQQQQVERQPDLPPGFRFHPTDEEIITFYLAPKVVDSRGFCVAAIGEVDLNKCEPWDLPGKAKMNGEKEWYFYCQKDRKYPTGMRTNRATEAGYWKATGKDKEIFRNHHMLIGMKKTLVFYKGRAPKGDKTNWVMHEYRLADASPPQPPPPPSSAEPPRQDDWAVCRIFHKSSGIKKPVQVPMQMPMQMQMPVAHQVPAANYQQQMAMASASIIQVPMQMQMPSMSDQLQMLDDFSTGSLMAPPPPPPSYSTLPGFPLQINGGAQQFVGNPSMYYQQQQQQQQQQMDMAAGGFVVSEPSSLVVSPQDAADQNNAADISSVACNMDATIWKY

SEQ ID NO: 71 - protein, 503 aa, Oryza sativa
MVEARLPPGFRFHPRDDELVVDYLSGKLRSGDGGAASGGGAAGAGCPTPTLIDVDLNKCEPWDLPEIACIGGKEWYFYNLKDRKYARGQRTNRATESGYWKATGKDREITRKGSLVGMRKTLVFYRGRAPKGERTDWVMHEFRQELDHANHHHHLKVLAHRFRFQFALDCIISHSHASWQLDYMQEGWVLCRVFYKSRTEAVAAPTMESTLPPRYINGGTSRSPLPPLVDSSISFNHGGYEEVLPCFSSSHHQQPSPASMNASAAADDDQDYHHLSEGQRHYSDKKMMRDVQNDQVTTRFDGHLAVKREMSLKKDLSEDEQAAPNADAGGFSILLKYSVSKMTSLMKPIQRNISTLQEFLNQKKEAILEKVEIFTKLLLPSRLGSAVFQLCLEHLIKNHKVGISWDGIWELSDWEVADNEVVLKMVGQCSAPADSKSKDLKRLFDLLRPYYDQEGKDPHLFFEHLKFDFTDVLKTIVTDAKWEWFWKYLLNHVFVMPPTGNTY

SEQ ID NO: 72 - protein, 373 aa, Oryza sativa
MERCSVLGLGGGGGGGGRLDGELPPGFRFHPTDEELITYYLLRKVVDGSFNGRAIAEIDLNKCEPWELPEKAKMGEKEWYFYSLRDRKYPTGLRTNRATGAGYWKATGKDREIRSARTGALVGMKKTLVFYRGRAPKGQKTQWVMHEYRLDGTYAYHFLSSSTRDEWVIARIFTKPGVFPVVRKGRLGISGGGGDTSCFSDSTSASVGGGGGTSASSALRAPLAEASLFAAAAAPAVDGADSSNYGGGGGAGSATATANLVTGLELVPCFSTTAHMDASFGTGQYNPAPLAVEPPPPPPAFFPSLRSLQENLQLPLFLSGGMQAGVSSQPLSGGGAFHWQSGMDVKVEGAVGRAPPQMAVGPGQLDGAFAWGF

SEQ ID NO: 73 - protein, 304 aa, Oryza sativa
MSGMNSLSMVEARLPPGFRFHPRDDELVLDYLERKLLDGGVGGAAAAAAAVTIYGCPVMVDVDLNKCEPWDLPEIACVGGKEWYFYSLRDRKYATGQRTNRATESGYWKATGKDRPISRKGLLVGMRKTLVFYKGRAPKGKKTEWVMHEFRKEGQGDPMKLPLKEDWVLCRVFYKSRTTIAKLPTEGSYNNIDSVATTSLPPLTDNYIAFDQPGSMQNLEGYEQVPCFSNNPSQQPSSSMNVPLTSAMVDQEQNNMGRAIKDVLSQFTKFEGNVKREALQSNFSQDGFDYLAESGFTQMWNSLS

SEQ ID NO: 74 - protein, 300 aa, Oryza sativa
MSARGGVTMAGGGGGDRAPSSSSTAMISRLLPPGFRFRPTDGELVAHYLARKAADAGFTSAAIRDADLYRAEPWDLLPPPRCDAAAEEEEEEEERCGYFFCTRSFRWPSGTRTNRATATGYWKSTGKDKAVLHGGGGGGGRPVGVKKTLVFYRGRAPRGEKTSWVMHEYRLLHGGAAATASSSPTPTTVVARSEWVICRVFVRKTPDGNNDRGTTEHHLPSDDAHLRSSPAPANSVDGAGHASCSFFSGANESMAPSDHFNIGDDMILHGHDEEELLMMNCSSAFDLPELLDYESFSLDL

SEQ ID NO: 75 - protein, 356 aa, Sorghum bicolor
MSEVSVINQAEVEDAGAAGQLDLPPGFRFHPTDEEIISHYLTHKALNHRFISGVIGEVDLNKCEPWDLPGRAKMGEKEWYFFCHKDRKYPTGTRTNRATETGYWKATGKDKEIFRGRGILVGMKKTLVFYRGRAPRGEKTGWVMHEFRLEGKLPQPLPRSAKDEWAVCKVFNKELAARTEPMAAAVAGAELERVGSLGFLNELLDSAELPALIGADVDEVIDFKGPASTSGLHAGAPGTSYLPVKMEEHALLQQMQYQQQPPPMFYSSQYFSLPAMNSGDLPPAIRRYCKAEQQVVSSGQTASVVSPSRETGLSTDRNAAGGGYAEISSAVTPSSSHQFLPELDDPVLNLADLWKY

SEQ ID NO: 76 - protein, 333 aa, Sorghum bicolor
MASEVSSEINQNQGGEEETRLDLPPGFRFHPTDEEVVSHYLTHKALNSSFSCLVIADVDLNKIEPWDLPSKAKMGEKEWYFFCHKDRKYPTGMRTNRATASGYWKATGKDKEIFRGHRVLVGMKKTLVFYTGRAPHGGKTPWVMHEYRLEGSLPSNLRRGAKDEWAVCKVFNKDLAAKAGQMAPAHAVGGGMVRSDSLAFLDDLVFDNADLPPLIDSPYAEGGLIDFNKIAGGGASSSSIAAAGTNDSAGYQVIKAEAQLPAAANNSAGGGSYSYEYQQQAIRRLCKAEAEAPATLLLSPSRGGGESSTDMFHVDDLLQLDGFMDDYSNMWKF

SEQ ID NO: 77 - protein, 362 aa, Sorghum bicolor
MEHDVHHHHHQQPEEAMELPPGFRFHPTDEELITHYLARKAADPRFAPRAVGVADLNKCEPWDLPYRATMGEKEWYFFCVKDRKYPTGLRTNRATESGYWKATGKDREIFRGKALVGLKKTLVFYTGRAPRGGKTGWVMHEYRLHGKHAAAAAAASSSSSLIPSVRAGASKDDWVLCRVFKKSIEPPSSVAGGSKRSSSSVACMGMEDVVGPSRSMADDFAASTLPPLMDVSGGSGGNMSLSAAAAAAASIELTTPPAPHVTCFSNTLEGHFLTPPTCLLPSAAATASPFLASMAQYDGDAGVGGMVHELLQEAGGWYSKLGERERLSGGASQDTGVTSEVNPAEISSTRHHMDHEASFWGF

SEQ ID NO: 78 - protein, 356 aa, Sorghum bicolor
MEQELHRPMELPPGFRFHPTDEELITHYLARKVADARFAALAVGEADLNKCEPWDLPSLAKMGEKEWYFFCLKDRKYPTGLRTNRATEAGYWKATGKDKDIFRGNGGKVLVGSKKTLVFYTGRAPKGEKSGWVMHEYRLHGKLHGAIVPPKAAAGSKNEWVLCRVFKKSLVVGGAAAAAPAAGKRGAMEMSKMNDDMAAISHLPPLMDVSGAGANVNPAAAAHVTCFSDALEGHQFFNQQQTPPEADATDHLGLAASSPFLLSGFAHYGPLHHGATSLVQLLEGSVVYAAAGGGGLPDMSNKQQQQPVPAPPCKVGGERERLSLSQDTGLTSDVNPEISSSSGAQRFDHDHLCWGY

SEQ ID NO: 79 - protein, 397 aa, Sorghum bicolor
MVERSVKSEHGVDLFLPPGFRFHPTDEEVITSYLLQKFLNPSFAPHAIGEVDLNKCEPWDLPSKANMGEKEWYFFCHKDMKYPTGTRTNRATKEGYWKATGKDREIFKQPGRELVGMKKTLVFYMGRAPRGTKTNWVMHEFRLDGKSRHTNDSNSNLRFNPKDEWVVCKVHHKGGEEASSKKAGGGGGEEHYSSAGTPNVSSVEAGEGGDEFLVDSLLDYSSYFNSSLPLTPSTTTTTISAAAPPYNADLLYPVHTTTAAAAANAGLTTTTTTTSSRFVGLPTDGSNYSQHAAAVANSVAAATKNNDDDSSASWNMLRHAADNKAAMGTNYSLHHQAMVAKVLGSGCGVSPNFGAGLPPAGSSVAAAGIIAQNNVVPQQRLAGNYRGNYAAGYHTSK

SEQ ID NO: 80 - protein, 372 aa, Sorghum bicolor
MEEQQQQEMNTASIGGGLTLPPGFRFHPSDNEIVSIYLMNKVHNRGFTSTIIAEVDLNKTEPWDLPQEAKIGEKEWYFFYQKDRKYPSGLRANRATKGGYWKATGRDKEVDNTTQGVVLLIGMKKTLVFYRGKAPKGDKTNWVMHEYRLEGSSRLPDPASASSSASNVLAMKASASKDEWVVCRVFDKTTSIKKMTTPVYKVAMASAEIGQNQNNTPAIPIPMPLQQPLPVPMPMESPILRDFATCPVAPYFPNTGADMPPMMSSMEGIDGTSSLQINDTLFGNSIATPPQMDLYHHMGMGVAAVHMGIGVAGQMDMAATGTDGFDLATPMPPSMASQKDEQANVAKMWSMMSVAGPGSVNPSTERDDIWKY

SEQ ID NO: 81 - protein, 362 aa, Sorghum bicolor
MADQQQPQQKGMNTVHAGGLDLPPGFRFHPSDYEIVSHYLTNKVRNMDLTYTAFEEVDLKKTEPWNLPIKAKWGEKEWYFFYLKDRKYSTGLRANRATKAGYWKATGKDKEVYNTIEGVVVLVGMKKTLVFYKGKAPRGDKTNWVMHEYRLEGSNSLSSPASTSNSADNVVTTMKASASAFKVVTIGIKKTTAPAYQVAMDGAEIDQRNINPTIPIPMPVQLPLSMSMPMQFPSLPDFTMDLMPPCYPNTSAEMSPMVGIGGGLQINDALFDNSIAALPQMNFYNQMGMEATADQMDMGVVADQMGKGGGFDVAMLESRPSSMVLERDEQANATEISSMLSMTGLGSVTTTIEMNDTWKYKY

SEQ ID NO: 82 - protein, 143 aa, Sorghum bicolor
MADQQQQQEMNDVCASGLNLPIGFHFDPSDFEIVNHFIRNKVRNRDYTCTAIGEVDLNKTEPWDLLKEAKMGEKEWYFFYQKDRKYPTGLRVNWATKAGYWKATGKDKKVYKPSKGEEVVLLVGMKKTLVFYKSRAPRGDKTN

SEQ ID NO: 83 - protein, 400 aa, Sorghum bicolor
MAEHHQPQQQEMNVVPTSGLDLPPGFRFHPSDIEIISDYLMNKVRNTNFTCIAIGEVDVNKTEPWDLPDQAKWGEKEWYFFNQKDRKYPTGLRANRATKAGYWKATGKDKEVYKTTEGVLMLVGMKKTLVFYKGRAPRGDKTNWVMHEYRLEGSGRLLGPASASSSIATAATATKASTSAPMDEWVVCRVFEKTTGIKKTTAPSYEVAMASTEIDQNQNNIQAIPIPMSLQLPLSVPMPMQFPIIPDFAMDPVAPYYPNASTGIPTMMPSMTGISGTSGLQINNAQFSNPIIASPQMNFYHQMGMGAVAGRIDMGAAVGQMGMGAGAGQMGMGAAVAQMDMGAAGAGGFDVVAPESRPSSMVSQKDDQANAAEISSMMSVTDPGSATTTIEMDGIWKYKY

SEQ ID NO: 84 - protein, 341 aa, Sorghum bicolor
MQHQVQDHHQAMGDTLWDLLGEEMAAAGGEHGLPPGFRFHPTDEELVTFYLAAKVFNGACCGIDIAEVDLNRCEPWELPDAARMGEREWYFFSLRDRKYPTGLRTNRATGAGYWKATGKDREVLNAATGALLGMKKTLVFYKGRAPRGEKTKWVLHEYRLDGDFAAARRPCKEEWVICRILHKAGDLYSKLMMVKNPYYLPMAMEPSSFCFQQEPTAHPLTNPSGGCTIPLGLPFHHGHPGMQPPSPSNHGNKQVVFAGAAGCCMQQEPPNGSNSAVLPMPPFPPFTPIVAGKPAAPAPPPQVGVNAGPQEPPPPTWLEAYLQHSDGILYEMGPAAAPRGA

SEQ ID NO: 85 - protein, 376 aa, Sorghum bicolor
MEAGGGGGGGGGESKKKEESLPPGFRFHPTDEELITYYLRQKIADGSFTARAIAEVDLNKCEPWDLPEKAKLGEKEWYFFSLRDRKYPTGVRTNRATNAGYWKTTGKDKEIYTGQLPATPELVGMKKTLVFYKGRAPRGEKTNWVMHEYRLHSKSVPKSNKDEWVVCRVFAKSAGAKKYPSNNAHSRSSHHHHPYALDMVPPLLPTLLQHDPFARHHGHHHHHPYMTPADLAELARFARGTPGLHPHIQPHPGTSAAAAAYMNPAVAAAAPPSFTLSGSGLNLNLGASPAMPSPPQALHAMSMAMGGQTGNHHQVMAGEHQHQHHQQQMATAAGLGGCVIVPGADGGFGADAAGGRYQSLDVEQLVERYWPAGYQV

SEQ ID NO: 86 - protein, 393 aa, Sorghum bicolor
MERFGVLGTRLGLDGVGIGGGGGGGELPPGFRFHPTDEELITYYLLRKAVDGSFCGRAIAEIDLNKCEPWELPDKAKMGEKEWYFYSLRDRKYPTGLRTNRATVAGYWKATGKDREIRSGRSGALVGMKKTLVFYRGRAPKGHKTHWVMHEFRLDGTYAYHFLPTSTRDEWVIARVFQKPGEVPPARKQHHRLGGLSSAGESCFSDSTSASIGGGGGASASSAPRPLPLTVTDASSLSLFAANAAADGDTSSYCGGHGGGAANTGNKLVTGRELVPCFSTSTTTGAALDAAALGIGQPYNAAVPPPLAFEAPPLPTPAFFPNLRSSLQVQDNHLQLPLFLSAGGGGGLSGTLGGGALHHWPFAGMEVKVEGRSAPPQMAVGPGQLDGAFGWGF

SEQ ID NO: 87 - protein, 321 aa, Sorghum bicolor
MEGDGDGRRAAPGPLPPGFRFRPTDEELLTHYLAPKVADAGFDPAALREVDLYKAEPWDLLPAEGGEDGGGVGYFFCRRSVKFPSGLRTNRATRAGYWKSTGKDRVVVASRSSSSRGDDDCPLGVRKTLVFYRGRAPTGHKTSWIMHEYRLLHGHGYTSPVVHATAGAQSEWVICRMFMKKAPGETSQPEQEEAVLHPPLEDHMQPPPVDDCGGETPAPAAAASDSDHQHVNCFSNIALAMVPGDTNFHGIESMLQLNRHGHEELWMNYPESTYPPVAAASTSGSAEAALRLRDELVADSSFDLLPQLLDYDEAFPFLQDF

SEQ ID NO: 88 - protein, 364 aa, Sorghum bicolor
MMRGVEQQQRIEELALPPGFRFFPTDEELITCYLARKAMDASFTTAAIRDVDLYKTEPWDLPCEQQAAAGGDLQEGYFFCMRGSKSPSGVRARRATQLGYWKSTGKDKAVHSRSGRLVVGTRKTLVFYRGRAPRGEKTDWVMHEYAMGERRSSALLRGAQSEWVICRVFTRKHHPMTSDDRKLETEELAVVQGHHSPGHHHPLAAMEGDDGFDSEQEAAAPPAVAETQHTAAGSHLGSTQSMEGDHQQQHRQMAHDELLTTTMHHHGSSSCVVSPCSCWLNQHDDHHQLVGLGGAHSALLPIMQMQSVVDDADYYLPELLEYGGPLDTGGGEEDRRRRAETNFTSVIGSSDDLDGLYWYWDSGF

SEQ ID NO: 89 - protein, 515 aa, Sorghum bicolor
MEVLRDMHLPPGFGFHPSDPELISHYLKRKILGQKIEYDLIPEVDIYKHEPWDLPAKCNLPIKDNKWHFFASRDRKYPTGSRSNRATLAGYWKSTGKDRAIKLNKRTLGTKKTLVFHEGRPPSGRRTEWIMHEYYIDENECKVSPDMKDAFVLCRVTKRSDWALDNDNEVGNRNSHLEQLDDAATSVVSTVKPEDAAASVICPEESNHAATPVGSAELCNDVAQAAITPDSRSPNGGIELETWLEELLDPSPSFNLVADTGSAGVSLTEQCAESSNPGFMAPNIGPGHASPIQDGTDATDYLFTDDLPEDLYSMLYPGTDQFNDNIFLEQVGQEGIAFPTNQAYYMMGTDAYALPNNFENGTPNVELQLDQENDQMNLPNGNVDTGIAIRSRRATASPANISLAYGNIKMQVGIKRMVTSNSESINQTMKFTHNSGRRLDLRTDVEHQKKNTNNVISAKQSDAAKTEGHSNQGYLKGFKRCSSAGFKSYIFVAFFVVGVAAAAAALHYHRSGANL

SEQ ID NO: 90 - protein, 261 aa, Sorghum bicolor
MAPVGLPPGFRFHPTDEELVNYYLKRKIHGLKIELDIIPEVDLYKCEPWELADKSFLPSRDPEWYFFGPRDRKYPNGFRTNRATRAGYWKSTGKDRRVLHHAGRPIGMKKTLVYYRGRAPQGVRTDWVMHEYRLDDKDAEDTLPIQISRHDLHGLCIDHVHEDTYALCRVFKKNAICTEVDGLQAQCSMALLEGACQQLLTSASQEYQTPSPDVPVGSTSGGADDDADKDESWMQFISDDAWCSSTADGAEESTSCVALAS

SEQ ID NO: 91 - protein, 218 aa, Sorghum bicolor
MAPADLPPGFRFHPTDEELVNYYLKRKVHGLSIELDIIPEVDLYKCEPWELAEKSFLPSRDSEWYFFGPRDRKYPNGCRTNRATQAGYWKSTGKDRRINYQNRSIGMKKTLVYYKGRAPQGLRTNWVMHEYRIEESECENTMGIQDSYALCRVFKKNVALGEFQKQKQGECSSSQANEKQEQFTSVRDAGQSSGSNEHGKDNTWMQFIADDLWCNKTK

SEQ ID NO: 92 - protein, 307 aa, Sorghum bicolor
MAINSLSMVEARLPPGFRFHPRDDELVLDYLAKKLAGGAGVGGGPLVVSIYGCPAMVDVDLNKCEPWDLPEIACIGGKEWYFYSLRDRKYATGQRTNRATDSGYWKATGKDRPISRKGLLVGMRKTLVFYQGRAPKGKKTEWVMHEFRMEGQGDPMKLPFKEDWVLCRVFYKSRATVAKPPTESSSSFNIDAATTSLPPLIDNNYNISFDQPGSSVQNLEGYEQVPCFSSNPSQLSSSINAPLSSAATMADPEQHMGKSIIKDVLMSQFSRFEAGNVKREAPQSNFSQDGFEYLAESGFTQMWNSFN

SEQ ID NO: 93 - protein, 267 aa, Sorghum bicolor
MERPAQAPTQLPPGFRFHPTDVELVVLYLRRQALARPLPAAVIPVVHDVARLDPWDLPGASEGEGYFFSLLRRAPATGRGSRRRRAGSGYWKATGKEKPVFLQCGGGVGGKGQLLVGVKTALAFHRSEPPAPSSRTGWIMHEYRLAVSRGVAEQREKNASQGCVAAPGEWVVCRVFLKNGARSRPNRDANSKALGHRASAAPPQPPPQHREDVGGRRQQPLLLSSSQSSSSSCVTGATDLADQDDEVSGGGGGISRDTPADPQREAY

SEQ ID NO: 94 - protein, 289 aa, Sorghum bicolor
MSSSISMMEARMPPGFRFHPRDDELVLDYLLDKLSGHGHGGGAAIVDVDLNKCEPWDLPESACVGGKEWYFFNLRDRKYATGQRTNRATRSGYWKATGKDRAVVAGGDGGEDAAAVVGMRKTLVFYRGRAPKGRKTEWVMHEFRLHPHAAPSLPAAATKEDWVLCRVFYKSRTTTPRPASDDAQDGTPSAEPQLSAALPLGPLADTYTAFGGAPTVFEQVSCFSGLPALPFKRPVSLGDLLAFDTSEKESIGTVMSSVSNNSSSVLELTPNCNWNQENDMLQMWNPLGI

SEQ ID NO: 95 - protein, 309 aa, Sorghum bicolor
MSLISMMEARLPPGFRFHPRDDELVLDYLCRKLSGKGGGGGGASYGGIAMVDVDLNKCEPWDLPGEACVGGREWYFFSLHDRKYATGQRTNRATRSGYWKATGKDRPISISGRRRGGGGNGAGALVGMRKTLVFYQGRAPRGTKTEWVMHEFRVDGPAVADRPGSPLQEDWVLCRVFYKSRTTTTRPAAGPDEAGPLSSELIGLPMPQMAPAGDAYLSFDNTPAADGGYYYHHQDADLADAHHHLPLPASSLSSFRDLLSSMVEGSDAAVRGTTELHLQGWTEAAYAQQQGGVMSSHSQQTWNPFLSSG

SEQ ID NO: 96 - protein, 303 aa, Sorghum bicolor
MGLRDIELTLPPGFRFYPSDEELVCHYLHGKVANERLAGAGAAMVEVDLHTHEPWELPDVAKLSTNEWYFFSFRDRKYATGLRTNRATKSGYWKATGKDRVIHNPRAGGHHHHRAIVGMRKTLVFYRGRAPNGIKTSWVMHEFRMENPHTPPKEDWVLCRVFYKKKADAMDYAMADSEQDVGMHMPRGGADSSSYSPPPFPALGGSHFHHHHLTPLTDHHGGGCLNNEFPGMAALLQHNNGMFDPHVVQPHLHDSVVLAGPPAAAAAAGSTRDGGEQCGSGVLMDLGLDEHYTYNSYNSLLQM

SEQ ID NO: 97 - protein, 302 aa, Sorghum bicolor
MALREIESTLPPGFRFYPSDEELVCHYLHKKVANERIAQGTLVEVDLHAREPWELPEVAKLTATEWYFFSFRDRKYATGSRTNRATKTGYWKATGKDREVRSSSSSGAVVGMRKTLVFYRGRAPNGVKSGWVMHEFRLDTPHSPPREDWVLCRVFQKTKGDGGDGQDGDYSSSSPAFAGTSQAMPDPDYYSAASSAGSGYYGLAFAPPPPQQQEEVAAPVLPQYYYGGTVDVDHHHHHHGFTRDDNVGAALPGFGGAMMRGSSVAAGGDQYGFGYFDMGGGGFDDMASFGGGDMDFVPQVWS

SEQ ID NO: 98 - protein, 341 aa, Sorghum bicolor
MEEGLPPGFRFHPTDEELVTYYLTRKVSDFAFATRAIADVDLNKCEPWDLPSKASMGEKEWYFFSMRDRKYPTGIRTNRATDSGYWKTTGKDKEIFHCGMLVGMKKTLVFYRGRAPKGQKTSWVMHEYRLQNKFPYKPNKEEWVVCRVFKKCQVIKMRPPQDSPTMGSPCHDAGNASLGELGELDVSSILGGLAAAAAHTSSGSPPGVLHHQGSAAAAESFVGAHNRPVDMSAYMSWMAAANQGAAAAAAMLPWATTTPPGLFGNVFAPNNHQLLQKPLPFAGGCSGQPRGLGGVVANNVVVGSGEHAMFGSSVVKVGMECDQQQQPEQQLGMDESTWRTF